(19) United States
(12) Patent Application Publication
(10) Pub. No.: US 2011/0301159 A1
(43) Pub. Date: Dec. 8, 2011

(54) KINASE INHIBITOR COMPOUNDS

(75) Inventors: Peter P. Sayeski, Gainesville, FL (US); Gyorgy M. Keseru, Budapest (HU); Kirpal Bisht, Tampa, FL (US)

(73) Assignees: UNIVERSITY OF SOUTH FLORIDA, Tampa, FL (US); UNIVERSITY OF FLORIDA RESEARCH FOUNDATION, INC., Gainesville, FL (US)

(21) Appl. No.: 13/133,779

(22) PCT Filed: Dec. 9, 2009
(86) PCT No.: PCT/US09/67402

§ 371 Date: Aug. 29, 2011

Related U.S. Application Data
(60) Provisional application No. 61/201,406, filed on Dec. 9, 2008.

(51) Int. Cl. A61K 31/5377; C07D 401/10; C07D 413/10; A61P 35/00; A61K 31/4545; A61K 31/4025; A61P 7/00; C07C 217/58; A61K 31/137

(52) U.S. Cl. 514/231.8; 564/389; 546/191; 548/524; 544/87; 514/655; 514/316; 514/422

(57) ABSTRACT

The disclosure relates to novel compounds that are capable of modulating Jak2 kinase activities, compounds that have therapeutic use in treating or preventing a subject suffering from or susceptible to a Jak2 mediated disease or disorder, and methods of use and compositions thereof.

### RELATED APPLICATION

This application claims the benefit of and priority to U.S. Provisional Patent Application No. 61/201,406, filed Dec. 9, 2008, the contents of which are incorporated herein by reference in their entirety.

### STATEMENT OF RIGHTS TO INVENTIONS MADE UNDER FEDERALLY SPONSORED RESEARCH

This work was supported in part by a National Institutes of Health/NHLBI Grant, Grant No. R01-HL67277. The government has certain rights in the invention.

### BACKGROUND OF THE INVENTION

Protein kinases constitute a large family of structurally related enzymes that are responsible for the control of a variety of signal transduction processes within the cell. (See, Hardie, G. and Hanks, S. The Protein Kinase Facts Book, I and II, Academic Press, San Diego, Calif.: 1995). The kinases may be categorized into families by the substrates they phosphorylate (e.g., protein-tyrosine, protein-serine/threonine, lipids, etc.). Sequence motifs have been identified that generally correspond to each of these kinase families (See, for example, Hanks, S. K., Hunter, T., FASEB J. 1995, 9, 576-596; Knighton et al., Science 1991, 253, 407-414; Hiles et al., Cell 1992, 70, 419-429; Kunz et al., Cell 1993, 73, 585-596; Garcia-Bustos et al., EMBO J. 1994, 13, 2352-2361).

In general, protein kinases mediate intracellular signaling by effecting a phosphoryl transfer from a nucleoside triphosphate to a protein acceptor that is involved in a signaling pathway. These phosphorylation events act as molecular on/off switches that can modulate or regulate the target protein biological function. These phosphorylation events are ultimately triggered in response to a variety of extracellular and other stimuli (e.g., environmental stress, chemical stress, signaling by agents including e.g., cytokines and growth factors).

The Janus kinases (JAK) are a family of tyrosine kinases consisting of Jak1, Jak2, Jak3 and TYK2. The JAKs play a critical role in cytokine signaling. The down-stream substrates of the JAK family of kinases include the signal transducer and activator of transcription (STAT) proteins. JAK/STAT signaling has been implicated in the mediation of many abnormal immune responses such as allergies, asthma, autoimmune diseases such as transplant rejection, rheumatoid arthritis, amyotrophic lateral sclerosis and multiple sclerosis as well as in solid and hematologic malignancies such as leukemias and lymphomas. The pharmaceutical intervention in the JAK/STAT pathway has been reviewed [Frank, Mol. Med. 5, 432-456 (1999) & Seidel et al., Oncogene 19, 2645-2656 (2000)].

Jak1, Jak2, and TYK2 are ubiquitously expressed, while Jak3 is predominantly expressed in hematopoietic cells. Jak3 binds exclusively to the common cytokine receptor gamma-chain and is activated by IL-2, IL-4, IL-7, IL-9, and IL-15. The proliferation and survival of murine mast cells induced by IL-4 and IL-9 have, in fact, been shown to be dependent on Jak3- and gamma-chain-signaling (Suzuki et al., Blood 96, 2172-2180 (2000)).

While certain known Jak2 inhibitor compounds have been proposed for therapeutic uses, these compounds often suffer limitations due, in part, to their lack of target specificity. As such, there is a need for therapeutic agents that are useful in mediating Jak2-mediated disease but are devoid of the side effect and selectivity limitations of existing agents.

### SUMMARY OF THE INVENTION

The invention provides compounds that can be used for treating Jak2-mediated diseases and disorders in a subject, and methods and uses thereof.

In one aspect, the invention relates to a compound of Formula (I):

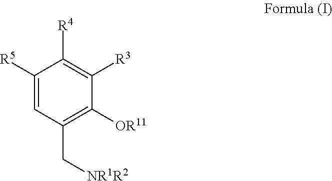

wherein
R¹ and R² are each independently H, —(C₁-C₄)alkyl, —(C₂-C₈)alkenyl, —(C₂-C₈)alkynyl,

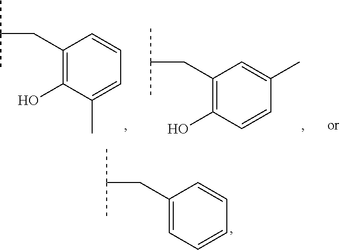

wherein —(C₁-C₄)alkyl can be further substituted with one or more hydroxy or halogen;
or
R¹ and R², together with the N-atom to which they are attached, to form a 5-membered or 6-membered heterocyclic ring, provided that when R¹ and R² together with the N-atom form a piperazine ring, the second nitrogen on the piperazine ring can be further optionally substituted with —(C₁-C₄)alkyl, —(C₃-C₇)cycloalkyl, aryl or acyl, wherein —(C₁-C₄)alkyl, —(C₃-C₇)cycloalkyl, aryl or acyl can be substituted with one or more hydroxy, halogen or —(C₁-C₃)alkyl;
R³ is H, —(C₁-C₄)alkyl, —(C₃-C₇)cycloalkyl, or aryl;

### R⁴ is H or R⁷;

R⁵ is H, —(C₁-C₄)alkyl, —C(CH₃)₂—R⁶, or R⁷; provided that when R⁴ is H, R⁵ is R⁷ or —C(CH₃)₂—R⁶, and that when R⁵ is H or —(C₁-C₄)alkyl, R⁴ is R⁷, wherein R⁴ and R⁵ cannot be both R⁷ at the same time;
R⁶ is H, —(C₁-C₄)alkyl, phenyl, or

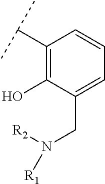

wherein R¹ and R² are as defined above;

### R⁷ is

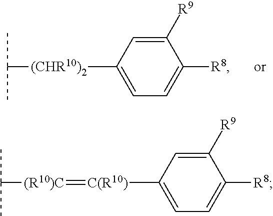

wherein R⁸ and R⁹ are each independently H, —OH, —O—(C₁-C₄)alkyl, —CH₂—NR¹R², wherein R¹ and R² are as defined above;
R¹⁰ for each occurrence independently is hydrogen, or —(C₁-C₃)alkyl;
R¹¹ is H, acyl, tosyl, —(C₁-C₄)alkyl, or aryl;
or a pharmaceutically acceptable salt, ester, hydrate or solvate thereof;
provided that the compound is not:
I. 4,4′-(Hex-3-ene-3,4-diyl)bis(2-((diethylamino)methyl)phenol);
II. 4,4′-(Ethene-1,2-diyl)bis(2-((diethylamino)methyl)phenol);
III. 5,5′-(Hex-3-ene-3,4-diyl)bis(2-((dimethylamino)methyl)phenol);
IV. 4,4′-(Hex-3-ene-3,4-diyl)bis(2-((dimethylamino)methyl)phenol);
V. 4,4′-(Ethene-1,2-diyl)bis(2-((dimethylamino)methyl)phenol);
VI. 4,4′-(Hex-3-ene-3,4-diyl)bis(2-((diethylamino)methyl)phenol);
VII. 4,4′-(Ethene-1,2-diyl)bis(2-((diethylamino)methyl)phenol); or
VIII. 4,4′-(Ethene-1,2-diyl)bis(2-(pyrrolidin-1-ylmethyl)phenol).

In one embodiment of the compounds of Formula (I), R¹¹ is hydrogen. In another embodiment, R¹⁰ for each occurrence independently is hydrogen, methyl or ethyl.

In one embodiment of the compounds of Formula (I), R³ is H. In another embodiment, one of R⁴ and R⁵ is R⁷. In a separate embodiment, R⁷ is

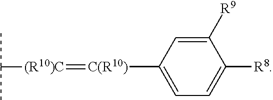

In one embodiment of the compounds of Formula (I), R⁴ is R⁷. In another embodiment, R⁵ is H. In one embodiment, R⁸ is —CH₂—NR¹R² and R⁹ is hydroxy, where R¹ and R² are defined in Formula (I). In one embodiment, R¹⁰ for each occurrence independently is hydrogen or methyl. In another embodiment, R¹ and R² for each occurrence independently are —(C₁-C₄)alkyl. In still another embodiment, R¹ and R² together with the N-atom to which they are attached form a piperidinyl, pyrrolidinyl or imidazolyl ring, wherein R¹⁰ is the same for each occurrence.

In one embodiment, R¹⁰ is ethyl. In another embodiment, R¹ and R² independently are ethyl or isopropyl. In another embodiment, R¹ and R² together with the N-atom to which they are attached form a pyrrolidinyl or imidazolyl ring.

In another embodiment, R⁴ is H. In certain embodiments, R⁵ is R⁷. In one embodiment, R⁸ is hydroxy and R⁹ is —CH₂—NR¹R², wherein R¹ and R² are defined in Formula (I). In one embodiment, R¹⁰ is methyl. In another embodiment, R¹ and R² for each occurrence independently are —(C₁₋₄)alkyl, or R¹ and R² together with the N-atom to which they are attached form a 5-membered or 6-membered heterocyclic ring. In another embodiment, R¹ and R² independently are propyl or isopropyl, when R¹⁰ is H or ethyl, and R¹⁰ is the same for each occurrence. In another embodiment, when R¹⁰ is ethyl, R¹ and R² together with the N-atom to which they are attached form a piperidinyl, pyrrolidinyl or imidazolyl ring.

In certain embodiments, the compound is selected from the group (Group (A)) consisting of
a) 4,4′-(but-2-ene-2,3-diyl)bis(2-((diethylamino)methyl)phenol);
b) 5,5′-(but-2-ene-2,3-diyl)bis(2-((diethylamino)methyl)phenol);
c) 5,5′-(but-2-ene-2,3-diyl)bis(2-((dimethylamino)methyl)phenol);
d) 5,5′-(ethene-1,2-diyl)bis(2-((diethylamino)methyl)phenol);
e) 5,5′-(ethene-1,2-diyl)bis(2-((dimethylamino)methyl)phenol);
f) 5,5′-(ethene-1,2-diyl)bis(2-(piperidin-1-ylmethyl)phenol);
g) 5,5′-(but-2-ene-2,3-diyl)bis(2-((diethylamino)methyl)phenol).2HCl;
h) 5,5′-(but-2-ene-2,3-diyl)bis(2-((dimethylamino)methyl)phenol).2HCl;
i) 5,5′-(ethene-1,2-diyl)bis(2-((diethylamino)methyl)phenol).2HCl;
j) 5,5′-(ethene-1,2-diyl)bis(2-((dimethylamino)methyl)phenol).2HCl;
k) 5,5′-(ethene-1,2-diyl)bis(2-(piperidin-1-ylmethyl)phenol).2HCl;
l) 5,5′-(but-2-ene-2,3-diyl)bis(2-((dimethylamino)methyl)phenol);
m) 5,5′-(ethene-1,2-diyl)bis(2-((dimethylamino)methyl)phenol);
n) 4,4′-(but-2-ene-2,3-diyl)bis(2-((dimethylamino)methyl)phenol);
o) 5,5′-(but-2-ene-2,3-diyl)bis(2-((diethylamino)methyl)phenol);
p) 5,5′-(hex-3-ene-3,4-diyl)bis(2-((diethylamino)methyl)phenol);
q) 5,5′-(ethene-1,2-diyl)bis(2-((diethylamino)methyl)phenol);
r) 4,4′-(but-2-ene-2,3-diyl)bis(2-((diethylamino)methyl)phenol);
s) 5,5′-(But-2-ene-2,3-diyl)bis(2-(pyrrolidin-1-ylmethyl)phenol);
t) 5,5′-(Hex-3-ene-3,4-diyl)bis(2-(pyrrolidin-1-ylmethyl)phenol);
u) 5,5′-(Ethene-1,2-diyl)bis(2-(pyrrolidin-1-ylmethyl)phenol);
v) 4,4′-(But-2-ene-2,3-diyl)bis(2-(pyrrolidin-1-ylmethyl)phenol);
w) 4,4′-(Hex-3-ene-3,4-diyl)bis(2-(pyrrolidin-1-ylmethyl)phenol);
x) 5,5′-(But-2-ene-2,3-diyl)bis(2-((diisopropylamino)methyl)phenol);
y) 5,5′-(Hex-3-ene-3,4-diyl)bis(2-((diisopropylamino)methyl)phenol);
z) 5,5′-(Ethene-1,2-diyl)bis(2-((diisopropylamino)methyl)phenol);
aa) 4,4′-(But-2-ene-2,3-diyl)bis(2-((diisopropylamino)methyl)phenol);
bb) 4,4′-(But-2-ene-2,3-diyl)bis(2-((diisopropylamino)methyl)phenol);
cc) 4,4′-(Hex-3-ene-3,4-diyl)bis(2-((diisopropylamino)methyl)phenol);
dd) 4,4′-(Ethene-1,2-diyl)bis(2-((diisopropylamino)methyl)phenol);
ee) 5,5′-(But-2-ene-2,3-diyl)bis(2-((1H-imidazol-1-yl)methyl)phenol);
ff) 5,5′-(Hex-3-ene-3,4-diyl)bis(2-((1H-imidazol-1-yl)methyl)phenol);
gg) 5,5′-(Ethene-1,2-diyl)bis(2-((1H-imidazol-1-yl)methyl)phenol);
hh) 4,4′-(But-2-ene-2,3-diyl)bis(2-(1H-imidazol-1-yl)methyl)phenol);
ii) 4,4′-(Hex-3-ene-3,4-diyl)bis(2-(1H-imidazol-1-yl)methyl)phenol); and
jj) 4,4′-(Ethene-1,2-diyl)bis(2-((1H-imidazol-1-yl)methyl)phenol);

or a pharmaceutically acceptable salt, ester, hydrate or solvate thereof.

The chemical name of each compound presented herein expressly encompasses both cis- and trans-isomers of the compound.

In another aspect, the invention relates to a compound of Formula (II):

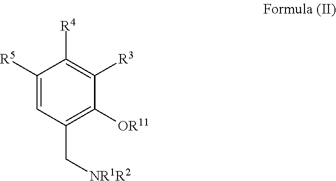

wherein

R¹ and R² are each independently H, —(C₁-C₄)alkyl, —(C₂-C₈)alkenyl, —(C₂-C₈)alkynyl,

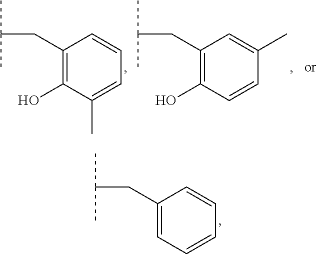

wherein —(C₁-C₄)alkyl can be further substituted with one or more hydroxy or halogen;
or
R¹ and R² together with the N-atom to which they are attached, to form a 5-membered or 6-membered heterocyclic ring, provided that when R¹ and R² together with the N-atom form a piperazine ring, the second nitrogen on the piperazine ring can be further optionally substituted with —(C₁-C₄)alkyl, —(C₃-C₇)cycloalkyl, aryl or acyl, wherein —(C₁-C₄)alkyl, —(C₃-C₇)cycloalkyl, aryl or acyl can be substituted with one or more hydroxy, halogen or —(C₁-C₃)alkyl;
R³ is H, —(C₁-C₄)alkyl, —(C₃-C₇)cycloalkyl, aryl;

### R⁴ is H or R⁷;

R⁵ is H, —(C₁-C₄)alkyl, —C(CH₃)₂—R⁶, or R⁷, provided that when R⁴ is H, R⁵ is R⁷ or —C(CH₃)₂—R⁶, and that when R⁵ is H or —(C₁-C₄)alkyl, R⁴ is R⁷, wherein R⁴ and R⁵ cannot be both R⁷ at the same time;
R⁶ is H, —(C₁-C₄)alkyl, phenyl, or

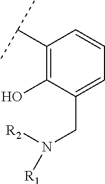

wherein R¹ and R² are as defined above;

### R⁷ is

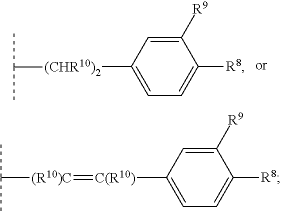

wherein R⁸ and R⁹ are each independently H, —OH, —O—(C₁-C₄)alkyl, —CH₂—NR¹R², wherein R¹ and R² are as defined above;
R¹⁰ for each occurrence independently is hydrogen, or —(C₁-C₃)alkyl;
R¹¹ is H, acyl, tosyl, —(C₁-C₄)alkyl, or aryl;
or a pharmaceutically acceptable salt, ester, hydrate or solvate thereof.

In certain embodiments, a compound of Formula (I) or Formula (II) is not a compound of the following group consisting of: 4,4′-(hex-3-ene-3,4-diyl)bis(2-((diethylamino)methyl)phenol) (“G6”); 4,4′-(hexane-3,4-diyl)bis(2-((diethylamino)methyl)phenol) (also as “D1); 4-benzyl-2-((diethylamino)methyl)phenol (also as “D2”); 2,2′-(methylazanediyl)bis(methylene)bis(4-methylphenol) (also as “D3”); 2-((dimethylamino)methyl)-4-(4-(4-hydroxyphenyl)hexan-3-yl)phenol (also as “D4”); 2,2′-(piperazine-1,4-diylbis(methylene))bis(4-ethylphenol) (also as “D5”); 2,2′-(piperazine-1,4-diylbis(methylene))bis(4-methylphenol) (also as “D6”); 6,6′-(methylazanediyl)bis(methylene)bis(2-methylphenol) (also as “D7”); 2,2′-(2-hydroxy-5-(4-(4-hydroxyphenyl)hex-3-en-3-yl)benzylazanediyl)diethanol (also as “D10”); 2-((dimethylamino)methyl)-4-(2-phenylpropan-2-yl)phenol (also as “D11”); 2-cyclohexyl-6-((diethylamino)methyl)-4-tert-pentylphenol (also as “D12”); 3-((diethylamino)methyl)-5-tert-pentylbiphenyl-2-ol (also as “D13”); 5-tert-butyl-3-((diethylamino)methyl)biphenyl-2-ol (also as “D14”); 3-((dimethylamino)methyl)biphenyl-2-ol (also as “D21”); 2-((diethylamino)methyl)-4-(4-(4-methoxyphenyl)hex-3-en-3-yl)phenol (also as “D22”); 2-((benzylamino)methyl)-4,6-dimethylphenol (also as “D23”); 2-cyclohexyl-6-((diethylamino)methyl)-4-(2-phenylpropan-2-yl)phenol (also as “D25”); 2-((dimethylamino)methyl)-4-(4-(4-methoxyphenyl)hex-3-en-3-yl)phenol (also as “D28”); 5,5′-(hexane-3,4-diyl)bis(2-((dimethylamino)methyl)phenol) (also as “D30”).

In certain embodiments, the invention provides a compound selected from the group (Group B) consisting of: 4,4′-(Hex-3-ene-3,4-diyl)bis(2-((diethylamino)methyl)phenol); 4,4′-(Ethene-1,2-diyl)bis(2-((diethylamino)methyl)phenol); 4,4′-(but-2-ene-2,3-diyl)bis(2-((diethylamino)methyl)phenol); 5,5′-(but-2-ene-2,3-diyl)bis(2-((diethylamino)methyl)phenol); 5,5′-(but-2-ene-2,3-diyl)bis(2-((dimethylamino)methyl)phenol); 5,5′-(ethene-1,2-diyl)bis(2-((diethylamino)methyl)phenol); 5,5′-(ethene-1,2-diyl)bis(2-((dimethylamino)methyl)phenol); 5,5′-(ethene-1,2-diyl)bis(2-(piperidin-1-ylmethyl)phenol); 5,5′-(but-2-ene-2,3-diyl)bis(2-((diethylamino)methyl)phenol).2HCl; 5,5′-(but-2-ene-2,3-diyl)bis(2-((dimethylamino)methyl)phenol).2HCl; 5,5′-(ethene-1,2-diyl)bis(2-((diethylamino)methyl)phenol).2HCl; 5,5′-(ethene-1,2-diyl)bis(2-((dimethylamino)methyl)phenol).2HCl; 5,5′-(ethene-1,2-diyl)bis(2-(piperidin-1-ylmethyl)phenol).2HCl; 5,5′-(but-2-ene-2,3-diyl)bis(2-((dimethylamino)methyl)phenol); 5,5′-(ethene-1,2-diyl)bis(2-((dimethylamino)methyl)phenol); 4,4′-(but-2-ene-2,3-diyl)bis(2-((dimethylamino)methyl)phenol); 5,5′-(but-2-ene-2,3-diyl)bis(2-((diethylamino)methyl)phenol); 5,5′-(hex-3-ene-3,4-diyl)bis(2-((diethylamino)methyl)phenol); 5,5′-(ethene-1,2-diyl)bis(2-((diethylamino)methyl)phenol); 4,4′-(but-2-ene-2,3-diyl)bis(2-((diethylamino)methyl)phenol); 5,5′-(But-2-ene-2,3-diyl)bis(2-(pyrrolidin-1-ylmethyl)phenol); 5,5′-(Hex-3-ene-3,4-diyl)bis(2-(pyrrolidin-1-ylmethyl)phenol); 5,5′-(Ethene-1,2-diyl)bis(2-(pyrrolidin-1-ylmethyl)phenol); 4,4′-(But-2-ene-2,3-diyl)bis(2-(pyrrolidin-1-ylmethyl)phenol); 4,4′-(Hex-3-ene-3,4-diyl)bis(2-(pyrrolidin-1-ylmethyl)phenol); 5,5′-(But-2-ene-2,3-diyl)bis(2-((diisopropylamino)methyl)phenol); 5,5′-(Hex-3-ene-3,4-diyl)bis(2-((diisopropylamino)methyl)phenol); 5,5′-(Ethene-1,2-diyl)bis(2-((diisopropylamino)methyl)phenol); 4,4′-(But-2-ene-2,3-diyl)bis(2-((diisopropylamino)methyl)phenol); 4,4′-(But-2-ene-2,3-diyl)bis(2-((diisopropylamino)methyl)phenol); 4,4′-(Hex-3-ene-3,4-diyl)bis(2-((diisopropylamino)methyl)phenol); 4,4′-(Ethene-1,2-diyl)bis(2-((diisopropylamino)methyl)phenol); 5,5′-(But-2-ene-2,3-diyl)bis(2-(1H-imidazol-1-yl)methyl)phenol); 5,5′-(Hex-3-ene-3,4-diyl)bis(2-(1H-imidazol-1-yl)methyl)phenol); 5,5′-(Ethene-1,2-diyl)bis(2-(1H-imidazol-1-yl)methyl)phenol); 4,4′-(But-2-ene-2,3-diyl)bis(2-((1H-imidazol-1-yl)methyl)phenol); 4,4′-(Hex-3-ene-3,4-diyl)bis(2-((1H-imidazol-1-yl)methyl)phenol); 4,4′-(Ethene-1,2-diyl)bis(2-((1H-imidazol-1-yl)methyl)phenol); 5,5′-(Hex-3-ene-3,4-diyl)bis(2-((dimethylamino)methyl)phenol); 4,4′-(Hex-3-ene-3,4-diyl)bis(2-((dimethylamino)methyl)phenol); 4,4′-(Ethene-1,2-diyl)bis(2-((dimethylamino)methyl)phenol); 4,4′-(Hex-3-ene-3,4-diyl)bis(2-((diethylamino)methyl)phenol); and 4,4′-(Ethene-1,2-diyl)bis(2-((diethylamino)methyl)phenol); and 4,4′-(Ethene-1,2-diyl)bis(2-(pyrrolidin-1-ylmethyl)phenol);

or a pharmaceutically acceptable salt, ester, hydrate or solvate thereof.

The name of each compound presented in Group (B) is meant to expressly encompass both cis- and trans-isomers of the compound.

In certain embodiments, the compound is selected from the following group (Group C):
1) (Z)- and (E)-4,4′-(Ethene-1,2-diyl)bis(2-((diethylamino)methyl)phenol) (“NB-1”):

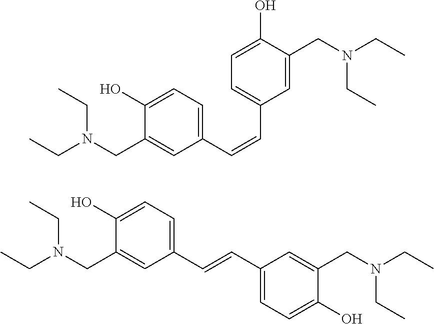

2) (Z)- and (E)-4,4′-(But-2-ene-2,3-diyl)bis(2-((diethylamino)methyl)phenol) (“NB-2”):

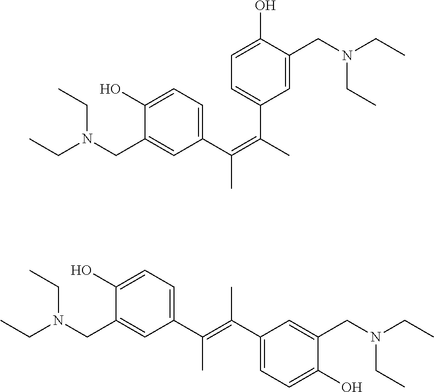

3) (Z)- and (E)-5,5′-(But-2-ene-2,3-diyl)bis(2-((diethylamino)methyl)phenol) (“NB-3”):

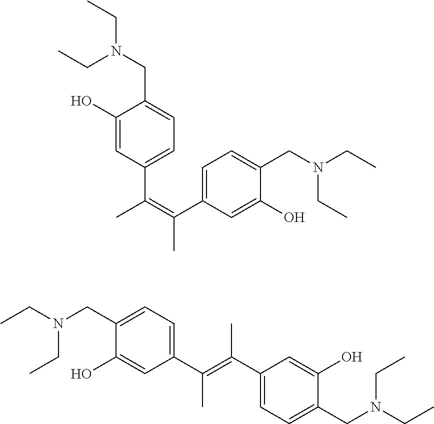

4) (Z)- and (E)-5,5′-(But-2-ene-2,3-diyl)bis(2-((dimethylamino)methyl)phenol) (“NB-4”):

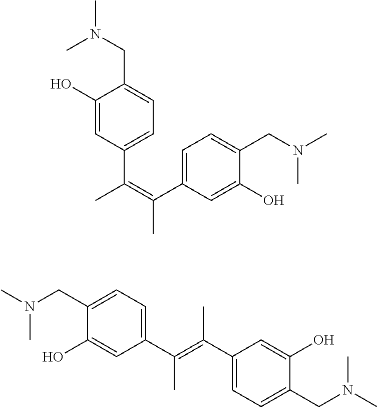

5) (Z)- and (E)-5,5′-(Ethene-1,2-diyl)bis(2-((diethylamino)methyl)phenol) (“NB-5”):

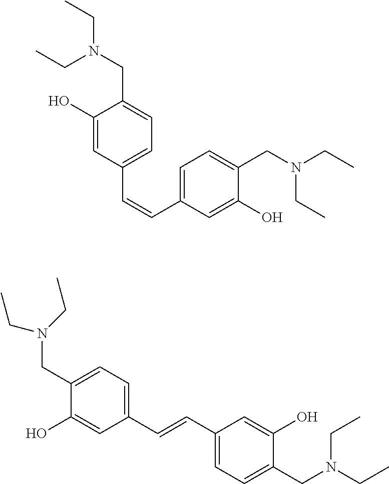

6) (Z)- and (E)-5,5′-(Ethene-1,2-diyl)bis(2-((dimethylamino)methyl)phenol) (“NB-6”):

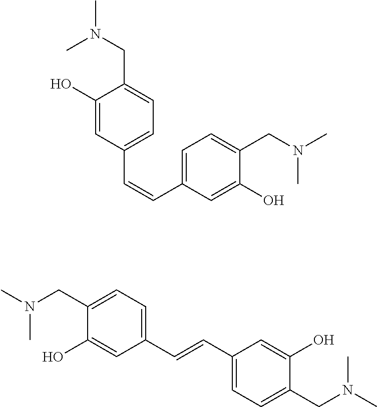

7) (Z)- and (E)-5,5′-(Ethene-1,2-diyl)bis(2-(piperidin-1-ylmethyl)phenol) (“NB-7”):

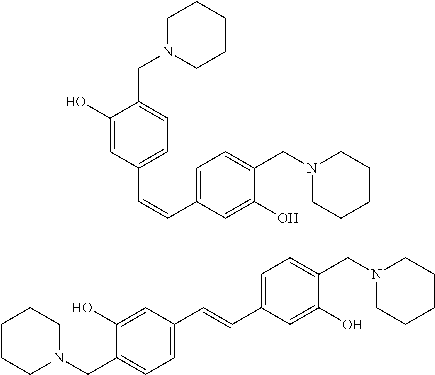

8) (Z)- and (E)-5,5′-(But-2-ene-2,3-diyl)bis(2-((diethylamino)methyl)phenol).2HCl (“NB-8”):

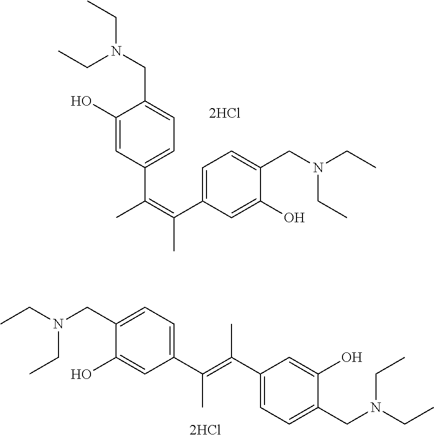

9) (Z)- and (E)-5,5′-(But-2-ene-2,3-diyl)bis(2-((dimethylamino)methyl)phenol).2HCl (“NB-9”):

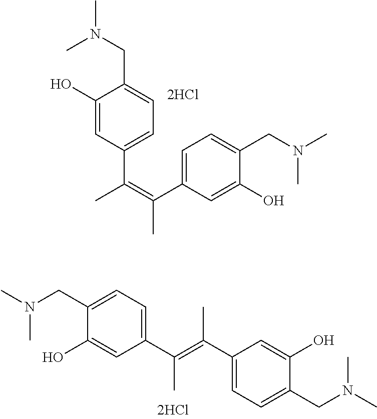

10) (Z)- and (E)-5,5′-(Ethene-1,2-diyl)bis(2-((diethylamino)methyl)phenol).2HCl (“NB-10”):

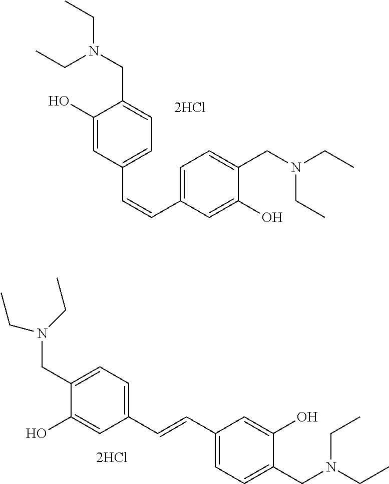

11) (Z)- and (E)-5,5′-(Ethene-1,2-diyl)bis(2-((dimethylamino)methyl)phenol).2HCl (“NB-11”):

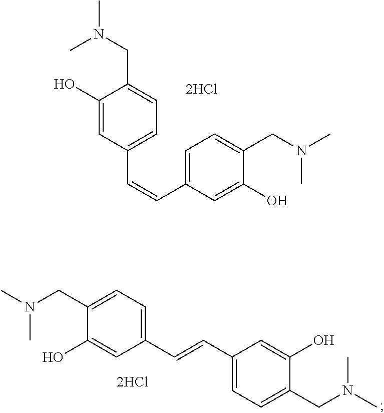

12) (Z)- and (E)-5,5′-(Ethene-1,2-diyl)bis(2-(piperidin-1-ylmethyl)phenol) (“NB-12”):

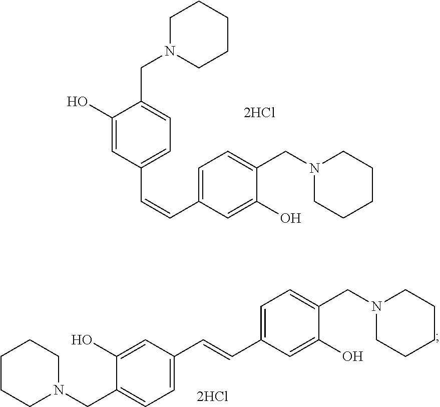

13) (Z) and (E)-4,4′-(But-2-ene-2,3-diyl)bis(2-((dimethylamino)methyl)phenol) (“NB-13”):

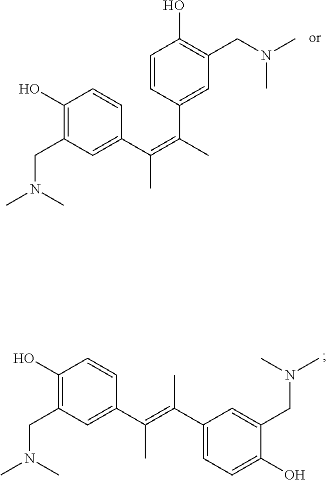

14) (Z) and (E)-5,5′-(Ethene-1,2-diyl)bis(2-(pyrrolidin-1-ylmethyl)phenol) (“NB-14”):

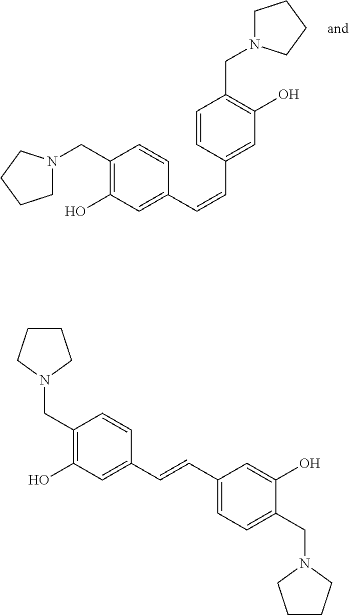

15) (Z) and (E)-4,4′-(Ethene-1,2-diyl)bis(2-((dimethylamino)methyl)phenol) (“NB-15”):

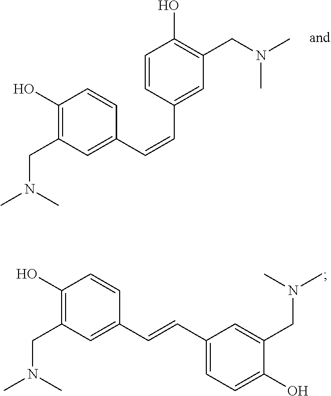

16) (Z) and (E)-5,5′-(But-2-ene-2,3-diyl)bis(2-(piperidin-1-ylmethyl)phenol) (“NB-16”):

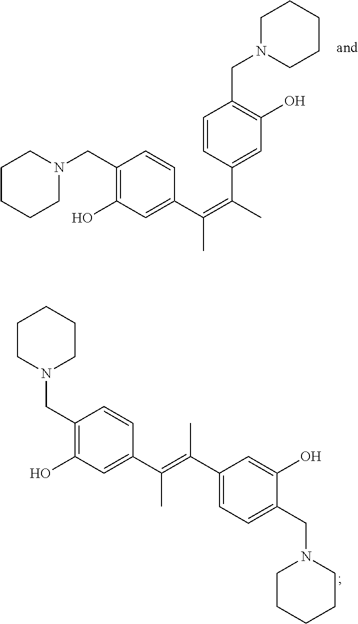

17) (Z) and (E)-(5,5′-(but-2-ene-2,3-diyl)bis(2-(pyrrolidin-1-ylmethyl)phenol) (“NB-17”):

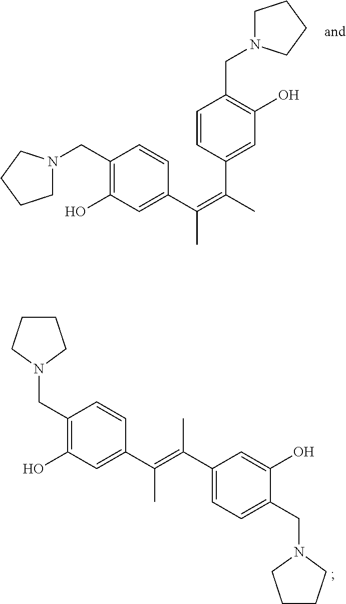

18) (Z) and (E)-5,5′-(Ethene-1,2-diyl)bis(2-(morpholinomethyl)phenol) (“NB-18”):

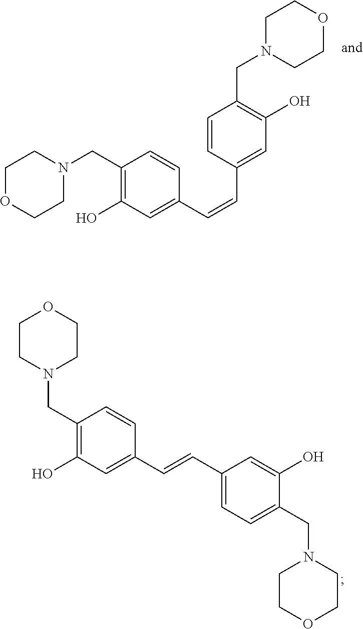

19) (Z) and (E)-5,5′-(But-2-ene-2,3-diyl)bis(2-(morpholinomethyl)phenol) (“NB-19”):

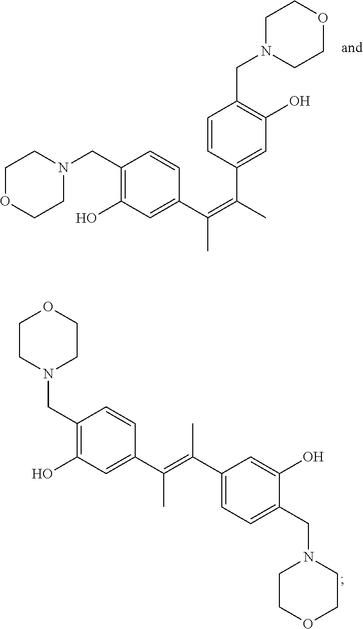

20) (Z) and (E)-4,4′-(Ethene-1,2-diyl)bis(2-(piperidin-1-ylmethyl)phenol) (“NB-20”):

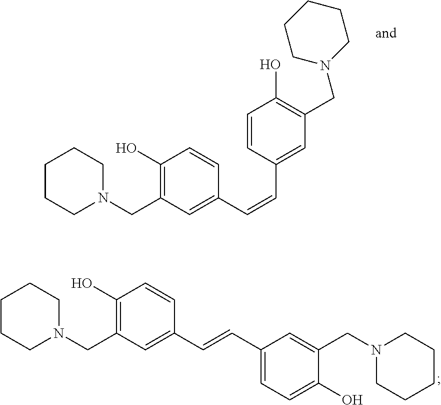

21) (Z) and (E)-4,4′-(Ethene-1,2-diyl)bis(2-(pyrrolidin-1-ylmethyl)phenol) (“NB-21”):

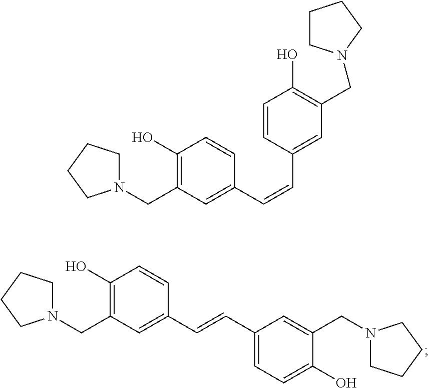

22) (Z) and (E)-4,4′-(Ethene-1,2-diyl)bis(2-(morpholinomethyl)phenol) (“NB-22”):

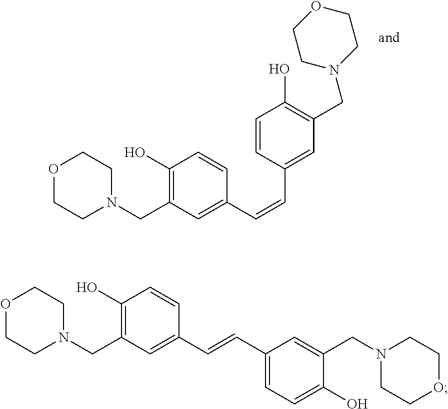

23) (Z) and (E)-4,4′-(Hex-3-ene-3,4-diyl)bis(2-((dimethylamino)methyl)phenol) (“NB-23”):

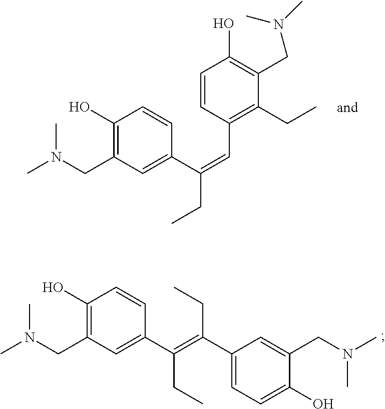

24) (Z) and (E)-4,4′-(Hex-3-ene-3,4-diyl)bis(2-(pyrrolidin-1-ylmethyl)phenol) (“NB-24”):

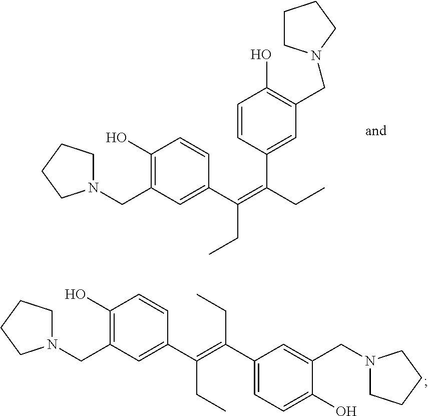

25) (Z) and (E)-4,4′-(Hex-3-ene-3,4-diyl)bis(2-(piperidin-1-ylmethyl)phenol) (“NB-25”):

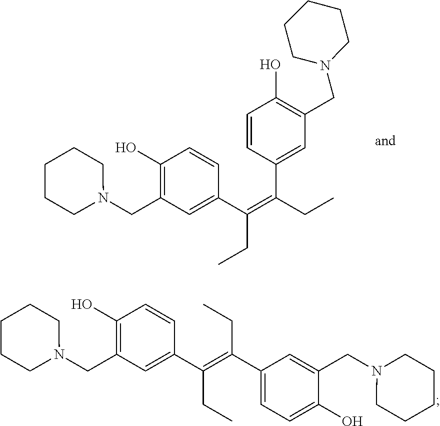

26) (Z) and (E)-4,4′-(Hex-3-ene-3,4-diyl)bis(2-(morpholinomethyl)phenol) (“NB-26”):

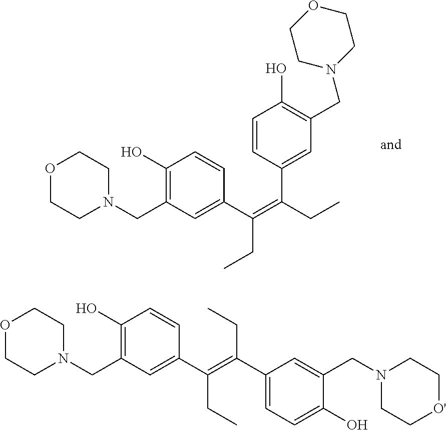

27) (Z) and (E)-4,4′-(But-2-ene-2,3-diyl)bis(2-(piperidin-1-ylmethyl)phenol) (“NB-27”):

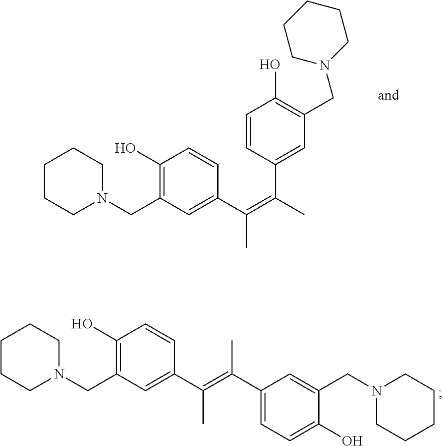

28) (Z) and (E)-4,4′-(But-2-ene-2,3-diyl)bis(2-(pyrrolidin-1-ylmethyl)phenol) (“NB-28”):

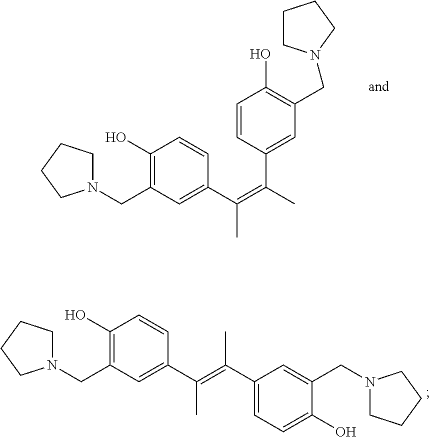

29) (Z) and (E)-2-((Diethylamino)methyl)-4-(4-(4-hydroxyphenyl)hex-3-en-3-yl)phenol (“NB-29”):

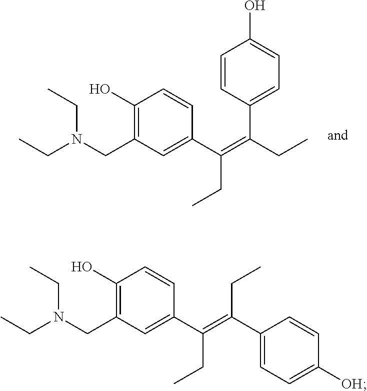

30) (Z) and (E)-4,4′-(Hex-3-ene-3,4-diyl)diphenol (“NB-30”)

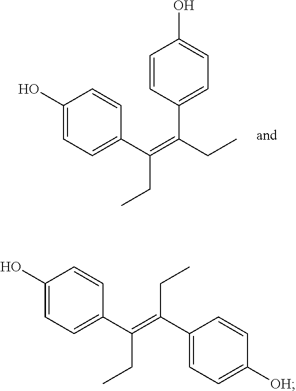

31) (Z) and (E)-4,4′-(But-2-ene-2,3-diyl)diphenol (“NB-31”):

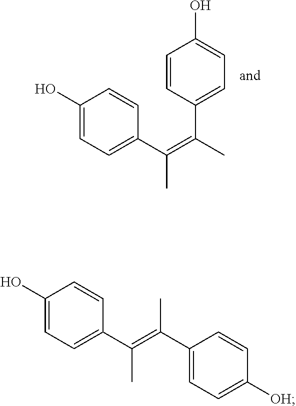

32) (Z) and (E)-3,3′-(Ethene-1,2-diyl)diphenol (“NB-32”):

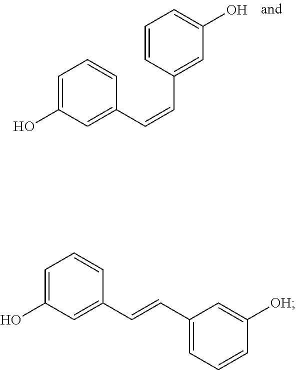

33) (Z) and (E)-3,3′-(But-2-ene-2,3-diyl)diphenol (“NB-33”):

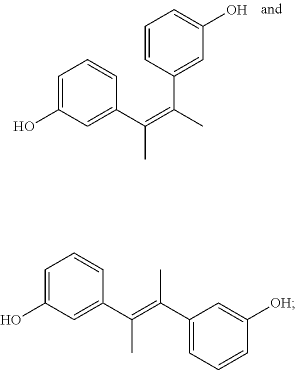

34) (Z) and (E)-4,4′-(Ethene-1,2-diyl)diphenol (“NB-34”):

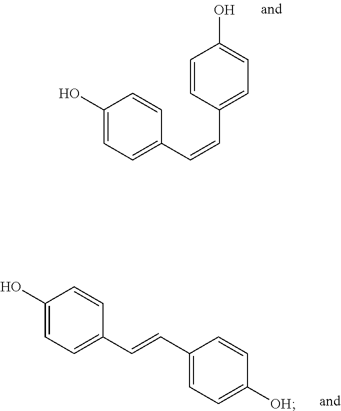

35) (Z) and (E)-4,4′-(Hex-3-ene-3,4-diyl)bis(2-((diethylamino)methyl)phenol) (“G6”):

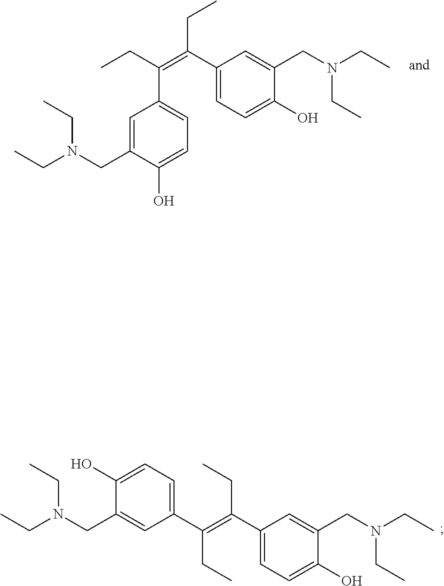

or a pharmaceutically acceptable salt, ester, hydrate or solvate thereof.

In still another embodiment, the compound is selected from the group (Group (D)) consisting of NB-1, NB-2, NB-3, NB-4, NB-5, NB-6, NB-7, NB-8, NB-9, NB-10, NB-11, and NB-12 (as above defined), or a pharmaceutically acceptable salt, ester, hydrate or solvate thereof.

Another aspect of the invention relates to a compound of Formula (III):

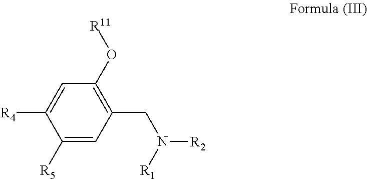

wherein
R¹ and R² are each independently H, —(C₁-C₄)alkyl, —(C₂-C₈)alkenyl, —(C₂-C₈)alkynyl,

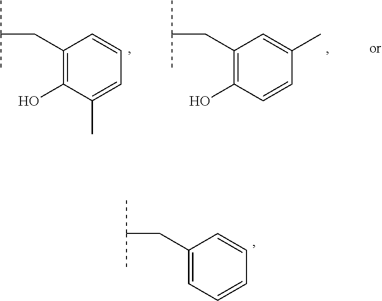

wherein —(C₁-C₄)alkyl can be further substituted with one or more hydroxy or halogen; or
R¹ and R², together with the N-atom to which they are attached, form a 5-membered or 6-membered heterocyclic ring, provided that when R¹ and R² together with the N-atom form a piperazine ring, the second nitrogen on the piperazine ring can be further optionally substituted with —(C₁-C₄)alkyl, —(C₃-C₇)cycloalkyl, aryl or acyl, wherein —(C₁-C₄)alkyl, —(C₃-C₇)cycloalkyl, aryl or acyl can be substituted with one or more hydroxy, halogen or —(C₁-C₃)alkyl;
R¹¹ is H, acyl, tosyl, —(C₁-C₄)alkyl, or aryl;
R⁴ and R⁵ are H or R¹², provided that one of R⁴ and R⁵ is H, and the other is R¹²;
R¹² is

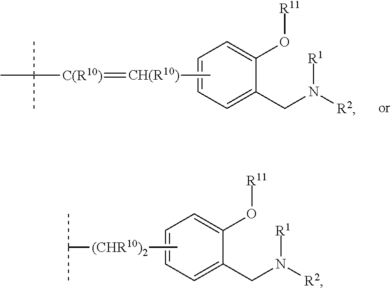

wherein the aryl group to which both R⁴ and R⁵ are attached is meta or para to the —OR¹¹ in the aromatic ring of R¹²;
R¹⁰ is hydrogen, or —(C₁-C₃)alkyl;
or a pharmaceutically acceptable salt, ester, hydrate or solvate thereof;
provided that the compound is not:
i. 4,4′-(Hex-3-ene-3,4-diyl)bis(2-((diethylamino)methyl)phenol); or
ii. 4,4′-(Ethene-1,2-diyl)bis(2-((diethylamino)methyl)phenol); or
iii. 5,5′-(Hex-3-ene-3,4-diyl)bis(2-((dimethylamino)methyl)phenol); or
iv. 4,4′-(Hex-3-ene-3,4-diyl)bis(2-((dimethylamino)methyl)phenol); or
v. 4,4′-(Ethene-1,2-diyl)bis(2-((dimethylamino)methyl)phenol); or
vi. 4,4′-(Hex-3-ene-3,4-diyl)bis(2-((diethylamino)methyl)phenol); or
vii. 4,4′-(Ethene-1,2-diyl)bis(2-((diethylamino)methyl)phenol); or
viii. 4,4′-(Ethene-1,2-diyl)bis(2-(pyrrolidin-1-ylmethyl)phenol).

The invention also provides a method for treating or preventing a Jak2 mediated disease or disorder in a subject. In certain embodiments, the method includes the step of administering to the subject an effective amount of a compound selected from Formulae (I), (II) and (III) as above defined, or a pharmaceutically acceptable salt, ester, hydrate or solvate thereof, such that the Jak2 mediated disease or disorder is treated or prevented in the subject. In certain embodiments, the compound administered to the subject is a compound of Formula (I) or Formula (III), or a pharmaceutically acceptable salt, ester, hydrate or solvate thereof.

In one embodiment, the compound is selected from Group (A), (B), (C) or (D) as above defined, or a pharmaceutically acceptable salt, hydrate or solvate thereof. In another embodiment, the compound is selected from Group (B), or a pharmaceutically acceptable salt, hydrate or solvate thereof. In another embodiment, the compound is a compound selected from Group (C) or a pharmaceutically acceptable salt, hydrate or solvate thereof. In still another embodiment, the compound is a compound of Group (D) or a pharmaceutically acceptable salt, hydrate or solvate thereof.

In one embodiment, the compound of the invention is administered to the subject at a dose between about 0.001 mg/Kg/day and about 200 mg/Kg/day, or between about 0.001 mg/Kg/day to about 30 mg/Kg/day. In certain embodiments, the compound of the invention is administered to the subject at a dose between about 0.1 mg/Kg/day and about 10 mg/Kg/day. In one embodiment, the compound is administered to the subject at a dose about 1 mg/Kg/day.

In one embodiment, the method also includes administering to the subject an additional therapeutic agent. In one embodiment, the compound of the invention and the additional therapeutic agent are administered simultaneously. In another embodiment, the compound of the invention and the additional therapeutic agent are administered sequentially.

In one embodiment, the Jak2-mediated disease or disorder is polycythemia vera, essential thrombocythemia, or angiogenic myeloid metaplasia. In another embodiment, the Jak2 mediated disorder is a cardiac disease or disorder. In certain embodiments, the cardiac disease or disorder is selected from the group of cardiac hypertrophy, cardiac ischemia-reperfusion, and heart failure.

In another embodiment, the compound is also an inhibitor of the Jak2-V617F mutant.

In another embodiment, the compound of Formula (I), Formula (II) or Formula (III) as above defined or a pharmaceutically acceptable salt, hydrate or solvate thereof inhibits Jak2 autophosphorylation. In another embodiment, the compound of Formulae (I), (II) and (III) as above defined, or a pharmaceutically acceptable salt, hydrate or solvate thereof does not inhibit c-Src or Tyk2 autophosphorylation as effectively as Jak2 autophosphorylation.

Yet in another embodiment, the subject is identified as having a Jak2-V617F mutant(s).

In another aspect, the invention provides a method of treating or preventing cancer in a subject. The method comprises administering to the subject an effective amount of a compound of Formula (I), (II) or (III), or a pharmaceutically acceptable salt, hydrate or solvate thereof, such that cancer is treated or prevented. In certain embodiments, the compound is a compound selected from Group (A), (B), (C) or (D) as above defined, a pharmaceutically acceptable salt, hydrate or solvate thereof. In one embodiment, the compound is a compound selected from Group (B), or a pharmaceutically acceptable salt, hydrate or solvate thereof. In another embodiment, the compound is a compound selected from Group (C), or a pharmaceutically acceptable salt, hydrate or solvate thereof. In still another embodiment, the compound is a compound selected from Group (D), or a pharmaceutically acceptable salt, hydrate or solvate thereof.

In one embodiment, the compound of the invention is administered to the subject at a dose between about 0.001 mg/Kg/day and about 200 mg/Kg/day, or between about 0.001 mg/Kg/day and about 30 mg/Kg/day. In certain embodiments, the compound of the invention is administered to the subject at a dose between about 0.1 mg/Kg/day and about 10 mg/Kg/day. In certain embodiments, the compound is administered to the subject at a dose about 1 mg/Kg/day.

In one embodiment, the cancer is selected from the group of leukemias, lymphomas, myelomas, and solid tumors. In another embodiment, the cancer is selected from the group of chronic myelogenous leukemia (CML), acute myeloid leukemia (AML), and acute promyelocytic leukemia (APL).

In another aspect, the invention provides a method for reducing Jak2-dependent cell growth. The method comprises contacting a cell (e.g., in vitro or in vivo, e.g., in a subject) with a Jak-2 inhibitor, wherein the inhibitor is a compound of Formula (I), (II) or (III) as above defined, or a pharmaceutically acceptable salt, hydrate or solvate thereof. In certain embodiments, the compound is selected from Group (A), (B), (C) or (D) as above defined, or a pharmaceutically acceptable salt, hydrate or solvate thereof. In one embodiment, the compound is a compound of Group (B), or a pharmaceutically acceptable salt, hydrate or solvate thereof. In certain embodiments, the compound is a compound of Group (C), or a pharmaceutically acceptable salt, hydrate or solvate thereof. Still another embodiment provides that the compound is a compound of Group (D), or a pharmaceutically acceptable salt, hydrate or solvate thereof.

In one embodiment, the compound of the invention is administered to the cell or subject at a dose between about 0.001 mg/Kg/day and about 200 mg/Kg/day, or between about 0.001 mg/Kg/day and about 30 mg/Kg/day. In certain embodiments, the compound of the invention is administered to the subject at a dose between about 0.1 mg/Kg/day and about 10 mg/Kg/day. In certain embodiments, the compound is administered to the subject at a dose about 1 mg/Kg/day.

Another aspect of the invention provides a method of inhibiting Jak2 in a subject identified as being in need of such treatment. The method comprises administering to the subject an effective amount of a compound of Formula (I), (II) or (III), or a pharmaceutically acceptable salt, hydrate or solvate thereof, such that Jak2 is inhibited in the subject. In certain embodiments, the compound is selected from Group (A), (B), (C) or (D) as above defined, a pharmaceutically acceptable salt, hydrate or solvate thereof. In certain embodiments, the compound is a compound of Group (C) or (D), or a pharmaceutically acceptable salt, hydrate or solvate thereof.

In one embodiment, the compound is administered to the subject identified as in need of treatment at a dose between about 0.001 mg/Kg/day and about 200 mg/Kg/day, or between about 0.001 mg/Kg/day and about 30 mg/Kg/day. In certain embodiments, the compound is administered to the subject at a dose between about 0.1 mg/Kg/day and about 10 mg/Kg/day. In certain embodiments, the compound is administered to the subject at a dose about 1 mg/Kg/day.

In another aspect, the invention provides a method of treating a hematological disease or disorder in a subject. The method comprises administering to the subject an effective amount of a compound of Formula (I), (II) or (III), or a pharmaceutically acceptable salt, hydrate or solvate thereof, such that the hematological disease or disorder is treated. In certain embodiments, the compound is selected from Group (A), (B), (C) or (D) as above defined, or a pharmaceutically acceptable salt, hydrate or solvate thereof. In certain embodiments, the compound is a compound selected from Group (C) or (D), or a pharmaceutically acceptable salt, ester, hydrate or solvate thereof.

The invention also provides a pharmaceutical composition, wherein the composition comprises a compound capable of modulating Jak2 activity, or a pharmaceutically acceptable ester, salt, or prodrug thereof, together with a pharmaceutically acceptable carrier. In one embodiment, the compound is a compound of Formula (II) or a pharmaceutically acceptable salt, ester, hydrate or solvate thereof. In certain embodiments, the compound is a compound of Formula (I) or (III) as above defined, or a pharmaceutically acceptable salt, ester, hydrate or solvate thereof. In certain embodiments, the compound is selected from Group (A), (B), (C) or (D) as above defined, or a pharmaceutically acceptable salt, hydrate or solvate thereof. In one embodiment, the compound is a compound of Group (B), or a pharmaceutically acceptable salt, ester, hydrate or solvate thereof. In another embodiment, the compound is a compound selected from Group (C), or a pharmaceutically acceptable salt, ester, hydrate or solvate thereof. In still another embodiment, the compound is a compound selected from Group (D), or a pharmaceutically acceptable salt, ester, hydrate or solvate thereof.

The invention also provides a kit for treating or preventing a Jak2-related disease or disorder in a subject. The kit includes at least one compound capable of modulating Jak2 activity, and instructions for use in treating or preventing the Jak2-related disease or disorder, wherein the compound is a compound of Formula (I), (II) or (III) as above defined, or a pharmaceutically acceptable salt, ester, hydrate or solvate thereof. In certain embodiments, the compound is selected from Group (A), (B), (C) or (D) as above defined, a pharmaceutically acceptable salt, ester, hydrate or solvate thereof. In one embodiment, the compound is a compound selected from Group (B), or a pharmaceutically acceptable salt, ester, hydrate or solvate thereof. In another embodiment, the compound is a compound selected from Group (C), or a pharmaceutically acceptable salt, ester, hydrate or solvate thereof. In yet another embodiment, the compound is a compound selected from Group (D), or a pharmaceutically acceptable salt, ester, hydrate or solvate thereof.

In one embodiment, the Jak2-related disease or disorder is selected from the group consisting of cancer, hematological disorders and cardiac disorders.

In another aspect, the invention provides a use of a compound of any of the formulae herein for the manufacture of a medicament. In certain embodiments, the medicament is a medicament for the treatment of a Jak2-related disease or disorder (e.g., cancer, a hematological disease or disorder, and the like).

The invention also provides methods for designing, evaluating and identifying compounds which bind to the binding pockets of Jak2. Other aspects and embodiments of the invention are disclosed infra.

### BRIEF DESCRIPTION OF THE DRAWINGS

The invention is further described below with reference to the following non-limiting examples and with reference to the following figures, in which:

FIG. 1 (a-f) depicts a Bone Marrow Analysis: 1(a) depicts untreated SCID 1, wherein the ratio of Myeloid cells:Erythoid cells is 1.11; 1(b) depicts 114 HEL cell having been treated with DMSO, wherein the ratio of Myeloid cells:Erythoid cells is 0.47; 1(c) depicts 234A HEL cells having been treated with 0.1 mg/kg G6, wherein the ratio of Myeloid cells:Erythoid cells is 0.3; 1(d) depicts 344 HEL cells having been treated with 1.0 mg/kg G6, wherein the ratio of Myeloid cells:Erythoid cells is 1.25; 1(e) depicts 444 HEL cells having been treated with 10 mg/kg G6, wherein the ratio of Myeloid cells:Erythoid cells is 1.2; 1(f) depicts 524SCID having been treated with 10 mg/kg G6, wherein the ratio of Myeloid cells:Erythoid cells is 1.1.

FIG. 2 depicts the results showing that G6 inhibits Jak2-V617F dependent HEL cell proliferation;

FIG. 3 depicts that the time required for G6 to inhibit Jak2-V617F dependent cell Proliferation by 50%.

FIG. 4 depicts the results showing that G6 inhibits Jak2-V617F dependent HEL cell proliferation in both a dose and time dependent manner;

FIG. 5 depicts the results showing that NB-1 inhibits Jak2-V617F dependent HEL cell proliferation;

FIG. 6 depicts the results showing that NB-2 inhibits Jak2-V617F dependent HEL cell proliferation;

FIG. 7 depicts the results showing that G6 has no effect on c-Src tyrosine kinase activity;

FIG. 8 depicts the results demonstrating that G6 reduces cell numbers by increasing cellular apoptosis;

FIG. 9 depicts the ex vivo results demonstrating that G6 blocks Jak2-V617F dependent megakaryocyte colony formation;

FIG. 10 depicts the ex vivo results demonstrating that G6, NB-1 and NB-2 reduce pathologic cell growth from a polycythemia vera patient, in a dose-dependent manner;

FIG. 11 depicts the in vivo results demonstrating that G6 reduces the percentage of blast cells in peripheral blood in a dose-dependent manner;

FIG. 12 is a graph depicting in vivo test results demonstrating that G6 reversed that HEL cell induced decrease in the ratio of Myeloid cells:Erythoid cells at a minimum dose of 1 mg/kg/day;

FIG. 13 is a graph depicting in vivo test results demonstrating that G6 treatment correlates with reduced numbers of mature Erythroid cells, not immature Erythroid cells;

FIG. 14 is a graph depicting in vivo test results demonstrating that G6 reduces the spleen weight to body weight ratio.

FIG. 15 depicts the results of a number of compounds tested in Jak2-V617F autophosphorylation assay.

### DETAILED DESCRIPTION OF THE INVENTION

The invention is directed to compounds with novel structures as defined in Formula (I). In certain embodiments, the compound is a compound of Formula (III). These compounds are capable of modulating Jak2 binding interactions. The invention also relates to compounds as defined in Formula (II) that can be used as inhibitors of Jak2 activities, and the compounds can also inhibit Jak2 mutants by targeting Jak2 interactions. The compounds of the invention are candidates as novel therapeutic drugs for treating or preventing Jak2-mediated disease or disorder, particularly in certain proliferation disease types where Jak2 and Jak2 mutants play a significant role.

The invention also relates, at least in part, to the discovery that the compounds delineated infra demonstrate selective interactions with certain targets (e.g., selective for Jak2 or Jak 2 mutants) for various disease therapies.

### 1. DEFINITIONS

Before further description of the invention, and in order that the invention may be more readily understood, certain terms are first defined and collected here for convenience.

As used in the specification and claims, the singular form “a”, “an” and “the” include plural references unless the context clearly dictates otherwise. For example, the term “a cell” includes a plurality of cells, including mixtures thereof. The term “a nucleic acid molecule” includes a plurality of nucleic acid molecules.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

The term “administration” or “administering” includes routes of introducing the compound of the invention to a subject to perform their intended function. Examples of routes of administration that may be used include injection (subcutaneous, intravenous, parenterally, intraperitoneally, intrathecal), oral, inhalation, rectal and transdermal. The pharmaceutical preparations may be given by forms suitable for each administration route. For example, these preparations are administered in tablets or capsule form, by injection, inhalation, eye lotion, ointment, suppository, etc. administration by injection, infusion or inhalation; topical by lotion or ointment; and rectal by suppositories. Oral administration is preferred. The injection can be bolus or can be continuous infusion. Depending on the route of administration, the compound of the invention can be coated with or disposed in a selected material to protect it from natural conditions which may detrimentally effect its ability to perform its intended function. The compound of the invention can be administered alone, or in conjunction with either another agent as described above or with a pharmaceutically-acceptable carrier, or both. The compound of the invention can be administered prior to the administration of the other agent, simultaneously with the agent, or after the administration of the agent. Furthermore, the compound of the invention can also be administered in a pro-drug form which is converted into its active metabolite, or more active metabolite in vivo.

The phrase “in combination with” is intended to refer to all forms of administration that provide an a compound of the invention (e.g. a compound selected from Formula (I), Formula (II) or Formula (III)) together with a second agent, such as a second compound selected from Formula (I), Formula (II) or Formula (III), or an existing therapeutic agent used for a particular disease or disorder, where the two are administered concurrently or sequentially in any order.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone, e.g., oxygen, nitrogen, sulfur or phosphorous atoms. In preferred embodiments, a straight chain or branched chain alkyl has 30 or fewer carbon atoms in its backbone (e.g., C₁-C₃₀ for straight chain, C₃-C₃₀ for branched chain), preferably 26 or fewer, and more preferably 20 or fewer, and still more preferably 4 or fewer. Likewise, preferred cycloalkyls have from 3-10 carbon atoms in their ring structure, and more preferably have 3, 4, 5, 6 or 7 carbons in the ring structure.

Moreover, the term alkyl as used throughout the specification and sentences is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate. Cycloalkyls can be further substituted, e.g., with the substituents described above. An “alkylaryl” moiety is an alkyl substituted with an aryl (e.g., phenylmethyl (benzyl)). The term “alkyl” also includes unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six, and still more preferably from one to four carbon atoms in its backbone structure, which may be straight or branched-chain. Examples of lower alkyl groups include methyl, ethyl, n-propyl, i-propyl, tert-butyl, hexyl, heptyl, octyl and so forth. In preferred embodiment, the term “lower alkyl” includes a straight chain alkyl having 4 or fewer carbon atoms in its backbone, e.g., C₁-C₄ alkyl.

The terms “alkoxyalkyl,” “polyaminoalkyl” and “thioalkoxyalkyl” refer to alkyl groups, as described above, which further include oxygen, nitrogen or sulfur atoms replacing one or more carbons of the hydrocarbon backbone, e.g., oxygen, nitrogen or sulfur atoms.

The terms “alkenyl” and “alkynyl” refer to unsaturated aliphatic groups analogous in length and possible substitution to the alkyls described above, but that contain at least one double or triple bond, respectively. For example, the invention contemplates cyano and propargyl groups.

The term “aryl” as used herein, refers to the radical of aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like. Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “associating with” refers to a condition of proximity between a chemical entity or compound, or portions thereof, and a binding pocket or binding site on a protein. The association may be non-covalent (wherein the juxtaposition is energetically favored by hydrogen bonding or van der Waals or electrostatic interactions) or it may be covalent.

The term “binding pocket”, as used herein, refers to a region of a molecule or molecular complex, that, as a result of its shape, favorably associates with another chemical entity or compound.

The language “biological activities” of a compound of the invention includes all activities elicited by compound of the invention in a responsive cell. It includes genomic and non-genomic activities elicited by these compounds.

“Biological composition” or “biological sample” refers to a composition containing or derived from cells or biopolymers. Cell-containing compositions include, for example, mammalian blood, red cell concentrates, platelet concentrates, leukocyte concentrates, blood cell proteins, blood plasma, platelet-rich plasma, a plasma concentrate, a precipitate from any fractionation of the plasma, a supernatant from any fractionation of the plasma, blood plasma protein fractions, purified or partially purified blood proteins or other components, serum, semen, mammalian colostrum, milk, saliva, placental extracts, a cryoprecipitate, a cryosupernatant, a cell lysate, mammalian cell culture or culture medium, products of fermentation, ascites fluid, proteins induced in blood cells, and products produced in cell culture by normal or transformed cells (e.g., via recombinant DNA or monoclonal antibody technology). Biological compositions can be cell-free. In a preferred embodiment, a suitable biological composition or biological sample is a red blood cell suspension. In some embodiments, the blood cell suspension includes mammalian blood cells. Preferably, the blood cells are obtained from a human, a non-human primate, a dog, a cat, a horse, a cow, a goat, a sheep or a pig. In preferred embodiments, the blood cell suspension includes red blood cells and/or platelets and/or leukocytes and/or bone marrow cells.

The term “chiral” refers to molecules which have the property of non-superimposability of the mirror image partner, while the term “achiral” refers to molecules which are superimposable on their minor image partner.

The term “diastereomers” refers to stereoisomers with two or more centers of dissymmetry and whose molecules are not mirror images of one another.

The term “effective amount” includes an amount effective, at dosages and for periods of time necessary, to achieve the desired result, e.g., sufficient to treat a disorder delineated herein. An effective amount of a compound of the invention may vary according to factors such as the disease state, age, and weight of the subject, and the ability of the compound of the invention to elicit a desired response in the subject. Dosage regimens may be adjusted to provide the optimum therapeutic response. An effective amount is also one in which any toxic or detrimental effects (e.g., side effects) of the compound of the invention are outweighed by the therapeutically beneficial effects.

The language “therapeutically effective amount” of a compound of the invention refers to an amount of an agent which is effective, upon single or multiple dose Jak2-mediated disorder, or in prolonging the survivability of the patient with such a Jak2-mediated disorder beyond that expected in the absence of such treatment.

A therapeutically effective amount of a compound of the invention (i.e., an effective dosage) may range from about 0.001 to about 100 mg/kg body weight, or about 0.1 to about 10 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound of the invention can include a single treatment or, preferably, can include a series of treatments. In one example, a subject is treated with a compound of the invention in the range of between about 0.1 to 100 mg/kg body weight, one time per week for between about 1 to 10 weeks. Certain examples are one time per week for between 2 to 8 weeks, and for between about 3 to 7 weeks. It will also be appreciated that the effective dosage of a compound of the invention used for treatment may increase or decrease over the course of a particular treatment.

By “agent” is meant a polypeptide, polynucleotide, or fragment, or analog thereof, small molecule, or other biologically active molecule.

The term “enantiomers” refers to two stereoisomers of a compound which are non-superimposable mirror images of one another. An equimolar mixture of two enantiomers is called a “racemic mixture” or a “racemate.”

The term “haloalkyl” is intended to include alkyl groups as defined above that are mono-, di- or polysubstituted by halogen, e.g., fluoromethyl and trifluoromethyl.

The term “halogen” designates —F, —Cl, —Br or —I.

The term “hydroxyl” means —OH.

The term “heteroatom” as used herein means an atom of any element other than carbon or hydrogen. Preferred heteroatoms are nitrogen, oxygen, sulfur and phosphorus.

The term “hematological disease or disorder” is meant to refer to a disease or disorder of the blood or blood forming tissues.

The term “cancer” is meant to refer to any disease that is caused by or results in inappropriately high levels of cell division, inappropriately low levels of apoptosis, or both. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphomas (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

The phrase “treating cancer” refers to the killing of malignant, or cancerous, cells. By treating is meant causing in the subject cell death in the tumor. Alternatively, “treating” cancer means arresting or otherwise ameliorating symptoms of cancer in the subject.

The language “improved biological properties” refers to any activity inherent in a compound of the invention that enhances its effectiveness in vivo. In a preferred embodiment, this term refers to any qualitative or quantitative improved therapeutic property of a compound of the invention, such as reduced toxicity.

The term “cell proliferative disorder” includes disorders involving the undesired or uncontrolled proliferation of a cell. Examples of such disorders include, but are not limited to, tumors or cancers (e.g., solid tumors such as breast, ovarian, prostate, lung (small cell and non-small cell), thyroid, pancreatic, breast or colon), sarcoma, leukemia, myeloma, lymphoma, or melanoma.

The term “optionally substituted” is intended to encompass groups that are unsubstituted or are substituted by other than hydrogen at one or more available positions, typically 1, 2, 3, 4 or 5 positions, by one or more suitable groups (which may be the same or different). Such optional substituents include, for example, hydroxy, halogen, cyano, nitro, C₁-C₈alkyl, C₂-C₈ alkenyl, C₂-C₈alkynyl, C₁-C₈alkoxy, C₂-C₈alkyl ether, C₃-C₈alkanone, C₁-C₈alkylthio, amino, mono- or di-(C1-C₈alkyl)amino, haloC₁-C₈alkyl, haloC₁-C₈alkoxy, C₁-C₈alkanoyl, C₂-C₈alkanoyloxy, C₁-C₈alkoxycarbonyl, —COOH, —CONH₂, mono- or di-(C₁-C₈alkyl)aminocarbonyl, —SO₂NH₂, and/or mono or di(C₁-C₈alkyl)sulfonamido, as well as carbocyclic and heterocyclic groups. Optional substitution is also indicated by the phrase “substituted with from 0 to X substituents,” where X is the maximum number of possible substituents. Certain optionally substituted groups are substituted with from 0 to 2, 3 or 4 independently selected substituents (i.e., are unsubstituted or substituted with up to the recited maximum number of substituents).

The term “isomers” or “stereoisomers” refers to compounds which have identical chemical constitution, but differ with regard to the arrangement of the atoms or groups in space.

The term “modulate” refers to an increase or decrease, e.g., in the ability of a cell to proliferate in response to exposure to a compound of the invention, e.g., the inhibition of proliferation of at least a sub-population of cells in an animal such that a desired end result is achieved, e.g., a therapeutic result. In certain preferred examples, the modulation is an inhibition. The term “inhibition” means decrease, suppress, attenuate, diminish, arrest, or stabilize the target activity, e.g. cell proliferation. In certain examples, the invention features compounds that modulate Jak2 activity.

The term “obtaining” as in “obtaining a compound” is intended to include purchasing, synthesizing or otherwise acquiring the compound.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “polycythemia vera” is meant to refer to a disease characterized by an abnormal increase in blood cells (primarily red blood cells) due to excess production of the cells by the bone marrow.

The term “essential thrombocythemia” is meant to refer to a blood disorder characterized by the overproduction of platelets by megakaryocytes in the bone marrow.

The term “primary myelofibrosis” is meant to refer to a disorder of the bone marrow, in which the marrow is replaced by fibrous (scar) tissue.

The term “prodrug” or “pro-drug” includes compounds with moieties that can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters. Prodrugs which are converted to active forms through other mechanisms in vivo are also included.

The language “a prophylactically effective amount” of a compound refers to an amount of a compound of the invention any formula herein or otherwise described herein which is effective, upon single or multiple dose administration to the patient, in preventing or treating a disorder delineated herein

The language “reduced toxicity” is intended to include a reduction in any undesired side effect elicited by a compound of the invention when administered in vivo.

The term “sulfhydryl” or “thiol” means —SH.

The term “subject” includes organisms which are capable of suffering from a Jak2-mediated disorder or who could otherwise benefit from the administration of a compound of the invention, such as human and non-human animals. Preferred humans include human patients suffering from or prone to suffering from a Jak2-mediated disorder, disorder delineated herein, or associated state, as described herein. The term “non-human animals” of the invention includes all vertebrates, e.g., mammals, e.g., rodents, e.g., mice, and non-mammals, such as non-human primates, e.g., sheep, dog, cow, chickens, amphibians, reptiles, etc.

The term “a Jak2-mediated disease or disorder” is meant to a disease or disorder mediated by or associated with Jak2 or a Jak2 mutant.

The term “susceptible to a Jak2-mediated disease or disorder” is meant to include subjects at risk of developing a Jak2-mediated disease/disorder, e.g., Jak2-mediated, i.e., subjects suffering from Jak2-mediated disease/disorder, subjects having a family or medical history of Jak2-mediated disease/disorder, and the like.

The phrases “systemic administration,” “administered systemically”, “peripheral administration” and “administered peripherally” as used herein mean the administration of a compound of the invention, drug or other material, such that it enters the patient's system and, thus, is subject to metabolism and other like processes, for example, subcutaneous administration.

With respect to the nomenclature of a chiral center, terms “d” and “1” configuration are as defined by the IUPAC Recommendations. As to the use of the terms, diastereomer, racemate, epimer and enantiomer will be used in their normal context to describe the stereochemistry of preparations.

### 2. COMPOUNDS

In one aspect, the invention provides a compound of Formula (I):

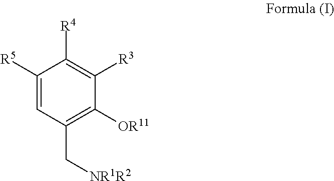

wherein
R¹ and R² are each independently H, —(C₁-C₄)alkyl, —(C₂-C₈)alkenyl, —(C₂-C₈)alkynyl,

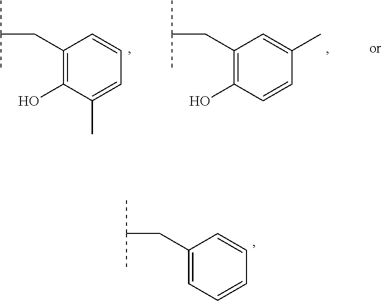

wherein —(C₁-C₄)alkyl can be further substituted with one or more hydroxy or halogen;
or
R¹ and R², together with the N-atom to which they are attached, to form a 5-membered or 6-membered heterocyclic ring, provided that when R¹ and R² together with the N-atom form a piperazine ring, the second nitrogen on the piperazine ring can be further substituted with —(C₁-C₄)alkyl, —(C₃-C₇)cycloalkyl, aryl or acyl, wherein —(C₁-C₄)alkyl, —(C₃-C₇)cycloalkyl, aryl or acyl can be substituted with one or more hydroxy, halogen or —(C₁-C₃)alkyl;
R³ is H, —(C₁-C₄)alkyl, —(C₃-C₇)cycloalkyl, aryl;

### R⁴ is H or R⁷;

R⁵ is H, —(C₁-C₄)alkyl, —C(CH₃)₂—R⁶, or R⁷, provided that when R⁴ is H, R⁵ is R⁷ or —C(CH₃)₂—R⁶, and that when R⁵ is H or —(C₁-C₄)alkyl, R⁴ is R⁷, wherein R⁴ and R⁵ cannot be both R⁷ at the same time;
R⁶ is H, —(C₁-C₄)alkyl, phenyl, or

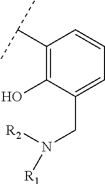

wherein R¹ and R² are as defined above;

### R⁷ is

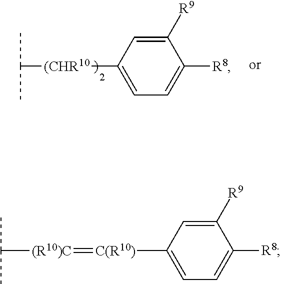

wherein R⁸ and R⁹ are each independently H, —OH, —O—(C₁-C₄)alkyl, —CH₂—NR¹R², wherein R¹ and R² are as defined above;
R¹⁰ for each occurrence is hydrogen, or —(C₁-C₃)alkyl;
R¹¹ is H, acyl, tosyl, —(C₁-C₄)alkyl, or aryl;
or a pharmaceutically acceptable salt, ester, hydrate or solvate thereof;
provided that the compound is not:
I. 4,4′-(Hex-3-ene-3,4-diyl)bis(2-((diethylamino)methyl)phenol); or
II. 4,4′-(Ethene-1,2-diyl)bis(2-((diethylamino)methyl)phenol); or
III. 5,5′-(Hex-3-ene-3,4-diyl)bis(2-((dimethylamino)methyl)phenol); or
IV. 4,4′-(Hex-3-ene-3,4-diyl)bis(2-((dimethylamino)methyl)phenol); or
V. 4,4′-(Ethene-1,2-diyl)bis(2-((dimethylamino)methyl)phenol); or
VI. 4,4′-(Hex-3-ene-3,4-diyl)bis(2-((diethylamino)methyl)phenol); or
VII. 4,4′-(Ethene-1,2-diyl)bis(2-((diethylamino)methyl)phenol); or
VIII. 4,4′-(Ethene-1,2-diyl)bis(2-(pyrrolidin-1-ylmethyl)phenol).

In one embodiment, R¹⁰ for each occurrence independently is hydrogen, methyl or ethyl. In another embodiment, R¹¹ is H.

In certain embodiments of the compounds of Formula (I), R³ is H. In another embodiment, one of R⁴ and R⁵ is R⁷. In a separate embodiment, R⁷ is

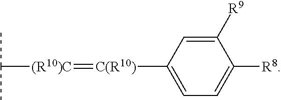

In one embodiment, R⁴ is R⁷. In another embodiment, R⁵ is H. In certain embodiments, R⁸ is —CH₂—NR¹R² and R⁹ is hydroxy, wherein R¹ and R² are defined in Formula (I). In one embodiment, R¹⁰ for each occurrence independently is hydrogen or methyl. In another embodiment, R¹ and R² for each occurrence independently are —(C₁-C₄)alkyl. In still another embodiment, R¹ and R² together with the N-atom to which they are attached to form a piperidinyl, pyrrolidinyl or imidazolyl ring, wherein R¹⁰ is the same for each occurrence.

In another embodiment, R¹⁰ is ethyl. In yet another embodiment, R¹ and R² for each occurrence independently are ethyl, or isopropyl. In certain embodiments, R¹ and R² together with the N-atom to which they are attached form a pyrrolidinyl or imidazolyl ring.

In another embodiment, R⁴ is H. In certain embodiments, R⁵ is R⁷. In one embodiment, R⁸ is hydroxy and R⁹ is —CH₂—NR¹R², wherein R¹ and R² are defined in Formula (I). In one embodiment, R¹⁰ is methyl. In certain embodiments, R¹ and R² for each occurrence independently are —(C₁₋₄)alkyl, or R¹ and R² together with the N-atom to which they are attached form a 5-membered or 6-membered heterocyclic ring. In another embodiment, R¹ and R² independently are propyl or isopropyl, when R¹⁰ is H or ethyl, and R¹⁰ is the same for each occurrence. In another embodiment, when R¹⁰ is ethyl, R¹ and R² together with the N-atom to which they are attached form a piperidinyl, pyrrolidinyl or imidazolyl ring.

In certain embodiments, the compound is selected from the following group (Group (A)):
a) 4,4′-(but-2-ene-2,3-diyl)bis(2-((diethylamino)methyl)phenol);
b) 5,5′-(but-2-ene-2,3-diyl)bis(2-((diethylamino)methyl)phenol);
c) 5,5′-(but-2-ene-2,3-diyl)bis(2-((dimethylamino)methyl)phenol);
d) 5,5′-(ethene-1,2-diyl)bis(2-((diethylamino)methyl)phenol);
e) 5,5′-(ethene-1,2-diyl)bis(2-((dimethylamino)methyl)phenol);
f) 5,5′-(ethene-1,2-diyl)bis(2-(piperidin-1-ylmethyl)phenol);
g) 5,5′-(but-2-ene-2,3-diyl)bis(2-((diethylamino)methyl)phenol).2HCl;
h) 5,5′-(but-2-ene-2,3-diyl)bis(2-((dimethylamino)methyl)phenol).2HCl;
i) 5,5′-(ethene-1,2-diyl)bis(2-((diethylamino)methyl)phenol).2HCl;
j) 5,5′-(ethene-1,2-diyl)bis(2-((dimethylamino)methyl)phenol).2HCl;
k) 5,5′-(ethene-1,2-diyl)bis(2-(piperidin-1-ylmethyl)phenol).2HCl;
l) 5,5′-(but-2-ene-2,3-diyl)bis(2-((dimethylamino)methyl)phenol);
m) 5,5′-(ethene-1,2-diyl)bis(2-((dimethylamino)methyl)phenol);
n) 4,4′-(but-2-ene-2,3-diyl)bis(2-((dimethylamino)methyl)phenol);
o) 5,5′-(but-2-ene-2,3-diyl)bis(2-((diethylamino)methyl)phenol);
p) 5,5′-(hex-3-ene-3,4-diyl)bis(2-((diethylamino)methyl)phenol);
q) 5,5′-(ethene-1,2-diyl)bis(2-((diethylamino)methyl)phenol);
r) 4,4′-(but-2-ene-2,3-diyl)bis(2-((diethylamino)methyl)phenol);
s) 5,5′-(But-2-ene-2,3-diyl)bis(2-(pyrrolidin-1-ylmethyl)phenol);
t) 5,5′-(Hex-3-ene-3,4-diyl)bis(2-(pyrrolidin-1-ylmethyl)phenol);
u) 5,5′-(Ethene-1,2-diyl)bis(2-(pyrrolidin-1-ylmethyl)phenol);
v) 4,4′-(But-2-ene-2,3-diyl)bis(2-(pyrrolidin-1-ylmethyl)phenol);
w) 4,4′-(Hex-3-ene-3,4-diyl)bis(2-(pyrrolidin-1-ylmethyl)phenol);
x) 5,5′-(But-2-ene-2,3-diyl)bis(2-((diisopropylamino)methyl)phenol);
y) 5,5′-(Hex-3-ene-3,4-diyl)bis(2-((diisopropylamino)methyl)phenol);
z) 5,5′-(Ethene-1,2-diyl)bis(2-((diisopropylamino)methyl)phenol);
aa) 4,4′-(But-2-ene-2,3-diyl)bis(2-((diisopropylamino)methyl)phenol);
bb) 4,4′-(But-2-ene-2,3-diyl)bis(2-((diisopropylamino)methyl)phenol);
cc) 4,4′-(Hex-3-ene-3,4-diyl)bis(2-((diisopropylamino)methyl)phenol);
dd) 4,4′-(Ethene-1,2-diyl)bis(2-((diisopropylamino)methyl)phenol);
ee) 5,5′-(But-2-ene-2,3-diyl)bis(2-((1H-imidazol-1-yl)methyl)phenol);
ff) 5,5′-(Hex-3-ene-3,4-diyl)bis(2-((1H-imidazol-1-yl)methyl)phenol);
gg) 5,5′-(Ethene-1,2-diyl)bis(2-((1H-imidazol-1-yl)methyl)phenol);
hh) 4,4′-(But-2-ene-2,3-diyl)bis(2-((1H-imidazol-1-yl)methyl)phenol);
ii) 4,4′-(Hex-3-ene-3,4-diyl)bis(2-((1H-imidazol-1-yl)methyl)phenol);
jj) 4,4′-(Ethene-1,2-diyl)bis(2-((1H-imidazol-1-yl)methyl)phenol);
   and a pharmaceutically acceptable salt, ester, hydrate or solvate thereof.

The name of each compound above-listed is meant to encompass both cis- and trans-isomers of the compound.

In another embodiment, the invention relates to a compound of Formula (II):

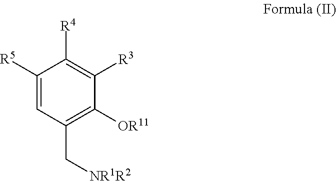

wherein
R¹ and R² are each independently H, —(C₁-C₄)alkyl, —(C₂-C₈)alkenyl, —(C₂-C₈)alkynyl,

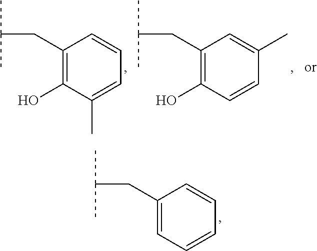

wherein —(C₁-C₄)alkyl can be further substituted with one or more hydroxy or halogen; or
R¹ and R² together with the N-atom to which they are attached, to form a 5-membered or 6-membered heterocyclic ring, provided that when R¹ and R² together with the N-atom form a piperazine ring, the second nitrogen on the piperazine ring can be further substituted with —(C₁-C₄)alkyl, —(C₃-C₇)cycloalkyl, aryl or acyl, wherein —(C₁-C₄)alkyl, —(C₃-C₇)cycloalkyl, aryl or acyl can be substituted with one or more hydroxy, halogen or —(C₁-C₃)alkyl;
R³ is H, —(C₁-C₄)alkyl, —(C₃-C₇)cycloalkyl, aryl;
R⁴ is H or R⁷;
R⁵ is H, —(C₁-C₄)alkyl, —C(CH₃)₂—R⁶, or R⁷, provided that when R⁴ is H, R⁵ is R⁷ or —C(CH₃)₂—R⁶, and that when R⁵ is H or —(C₁-C₄)alkyl, R⁴ is R⁷, wherein R⁴ and R⁵ cannot be both R⁷ at the same time;
R⁶ is H, —(C₁-C₄)alkyl, phenyl, or

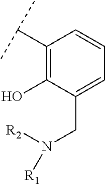

wherein R¹ and R² are as defined above;
R⁷ is

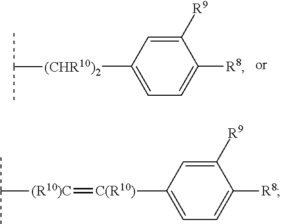

wherein R⁸ and R⁹ are each independently H, —OH, —O—(C₁-C₄)alkyl, —CH₂—NR¹R², wherein R¹ and R² are as defined above;
R¹⁰ for each occurrence independently is hydrogen, or —(C₁-C₃)alkyl;
R¹¹ is H, acyl, tosyl, —(C₁-C₄)alkyl, or aryl;
or a pharmaceutically acceptable salt, hydrate or solvate thereof.

In particular, the invention relates to a compound of Group B consisting of 4,4′-(Hex-3-ene-3,4-diyl)bis(2-((diethylamino)methyl)phenol), 4,4′-(Ethene-1,2-diyl)bis(2-((diethylamino)methyl)phenol), 4,4′-(but-2-ene-2,3-diyl)bis(2-((diethylamino)methyl)phenol), 5,5′-(but-2-ene-2,3-diyl)bis(2-((diethylamino)methyl)phenol), 5,5′-(but-2-ene-2,3-diyl)bis(2-((dimethylamino)methyl)phenol), 5,5′-(ethene-1,2-diyl)bis(2-((diethylamino)methyl)phenol), 5,5′-(ethene-1,2-diyl)bis(2-((dimethylamino)methyl)phenol), 5,5′-(ethene-1,2-diyl)bis(2-(piperidin-1-ylmethyl)phenol), 5,5′-(but-2-ene-2,3-diyl)bis(2-((diethylamino)methyl)phenol).2HCl, 5,5′-(but-2-ene-2,3-diyl)bis(2-((dimethylamino)methyl)phenol).2HCl; 5,5′-(ethene-1,2-diyl)bis(2-((diethylamino)methyl)phenol).2HCl; 5,5′-(ethene-1,2-diyl)bis(2-((dimethylamino)methyl)phenol).2HCl; 5,5′-(ethene-1,2-diyl)bis(2-(piperidin-1-ylmethyl)phenol).2HCl; 5,5′-(but-2-ene-2,3-diyl)bis(2-((dimethylamino)methyl)phenol); 5,5′-(ethene-1,2-diyl)bis(2-((dimethylamino)methyl)phenol); 4,4′-(but-2-ene-2,3-diyl)bis(2-((dimethylamino)methyl)phenol); 5,5′-(but-2-ene-2,3-diyl)bis(2-((diethylamino)methyl)phenol); 5,5′-(hex-3-ene-3,4-diyl)bis(2-((diethylamino)methyl)phenol); 5,5′-(ethene-1,2-diyl)bis(2-((diethylamino)methyl)phenol); 4,4′-(but-2-ene-2,3-diyl)bis(2-((diethylamino)methyl)phenol); 5,5′-(But-2-ene-2,3-diyl)bis(2-(pyrrolidin-1-ylmethyl)phenol); 5,5′-(Hex-3-ene-3,4-diyl)bis(2-(pyrrolidin-1-ylmethyl)phenol); 5,5′-(Ethene-1,2-diyl)bis(2-(pyrrolidin-1-ylmethyl)phenol); 4,4′-(But-2-ene-2,3-diyl)bis(2-(pyrrolidin-1-ylmethyl)phenol); 4,4′-(Hex-3-ene-3,4-diyl)bis(2-(pyrrolidin-1-ylmethyl)phenol); 5,5′-(But-2-ene-2,3-diyl)bis(2-((diisopropylamino)methyl)phenol); 5,5′-(Hex-3-ene-3,4-diyl)bis(2-((diisopropylamino)methyl)phenol); 5,5′-(Ethene-1,2-diyl)bis(2-((diisopropylamino)methyl)phenol); 4,4′-(But-2-ene-2,3-diyl)bis(2-((diisopropylamino)methyl)phenol); 4,4′-(But-2-ene-2,3-diyl)bis(2-((diisopropylamino)methyl)phenol); 4,4′-(Hex-3-ene-3,4-diyl)bis(2-((diisopropylamino)methyl)phenol); 4,4′-(Ethene-1,2-diyl)bis(2-((diisopropylamino)methyl)phenol); 5,5′-(But-2-ene-2,3-diyl)bis(2-((1H-imidazol-1-yl)methyl)phenol); 5,5′-(Hex-3-ene-3,4-diyl)bis(2-((1H-imidazol-1-yl)methyl)phenol); 5,5′-(Ethene-1,2-diyl)bis(2-((1H-imidazol-1-yl)methyl)phenol); 4,4′-(But-2-ene-2,3-diyl)bis(2-((1H-imidazol-1-yl)methyl)phenol); 4,4′-(Hex-3-ene-3,4-diyl)bis(2-((1H-imidazol-1-yl)methyl)phenol); 4,4′-(Ethene-1,2-diyl)bis(2-((1H-imidazol-1-yl)methyl)phenol); 5,5′-(Hex-3-ene-3,4-diyl)bis(2-((dimethylamino)methyl)phenol); 4,4′-(Hex-3-ene-3,4-diyl)bis(2-((dimethylamino)methyl)phenol); 4,4′-(Ethene-1,2-diyl)bis(2-((dimethylamino)methyl)phenol); 4,4′-(Hex-3-ene-3,4-diyl)bis(2-((diethylamino)methyl)phenol), 4,4′-(Ethene-1,2-diyl)bis(2-((diethylamino)methyl)phenol), and 4,4′-(Ethene-1,2-diyl)bis(2-(pyrrolidin-1-ylmethyl)phenol); or its pharmaceutically acceptable salt, hydrate or solvate thereof.

Unless otherwise provided, the chemical name of each compound herein is meant to expressly encompass both cis- and trans-isomers of the compound.

In certain embodiments, the invention provides a compound selected from the following group (Group C):
1) (Z)- and (E)-4,4′-(Ethene-1,2-diyl)bis(2-((diethylamino)methyl)phenol) (“NB-1”):

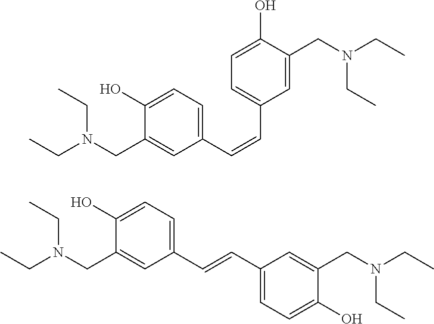

2) (Z)- and (E)-4,4′-(But-2-ene-2,3-diyl)bis(2-((diethylamino)methyl)phenol) (“NB-2”):

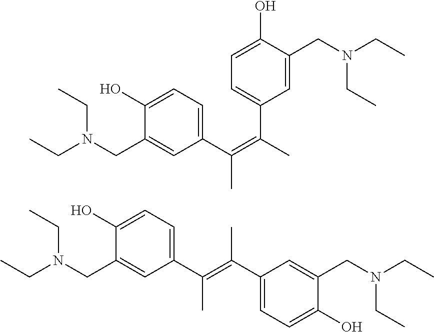

3) (Z)- and (E)-5,5′-(But-2-ene-2,3-diyl)bis(2-((diethylamino)methyl)phenol) (“NB-3”):

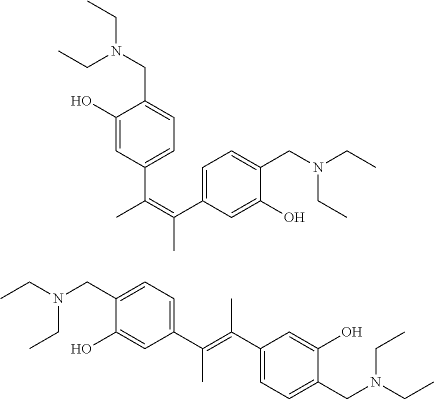

4) (Z)- and (E)-5,5′-(But-2-ene-2,3-diyl)bis(2-((dimethylamino)methyl)phenol) (“NB-4”):

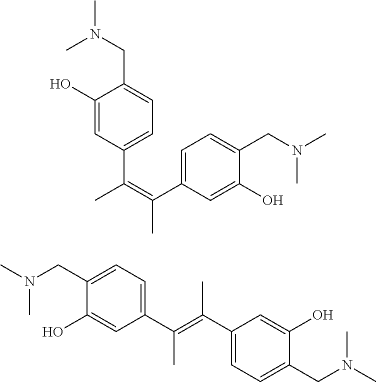

5) (Z)- and (E)-5,5′-(Ethene-1,2-diyl)bis(2-((diethylamino)methyl)phenol) (“NB-5”):

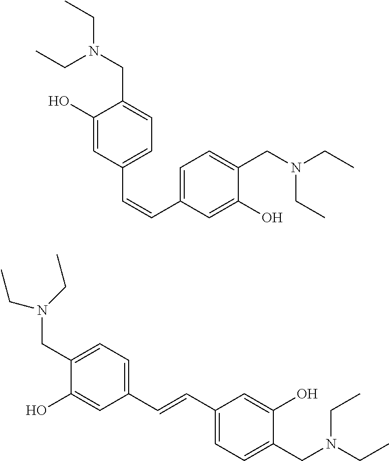

6) (Z)- and (E)-5,5′-(Ethene-1,2-diyl)bis(2-((dimethylamino)methyl)phenol) (“NB-6”):

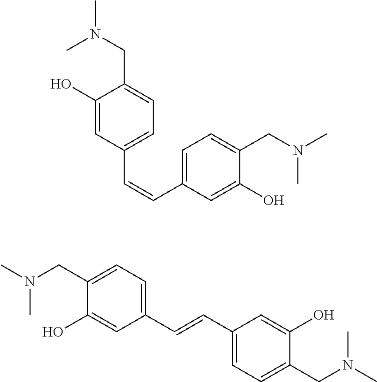

7) (Z)- and (E)-5,5′-(Ethene-1,2-diyl)bis(2-(piperidin-1-ylmethyl)phenol) (“NB-7”):

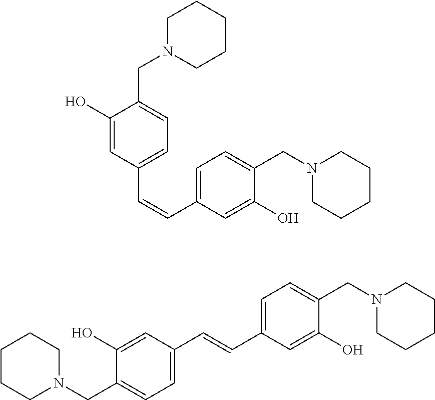

8) (Z)- and (E)-5,5′-(But-2-ene-2,3-diyl)bis(2-((diethylamino)methyl)phenol).2HCl (“NB-8”):

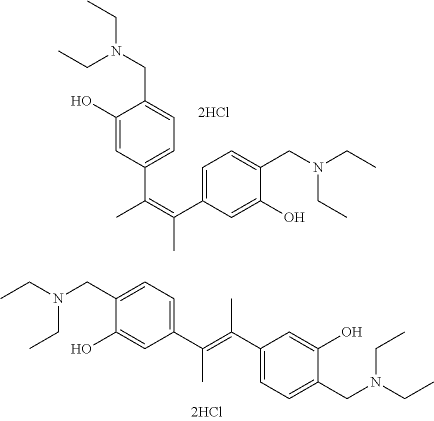

9) (Z)- and (E)-5,5′-(But-2-ene-2,3-diyl)bis(2-((dimethylamino)methyl)phenol).2HCl (“NB-9”):

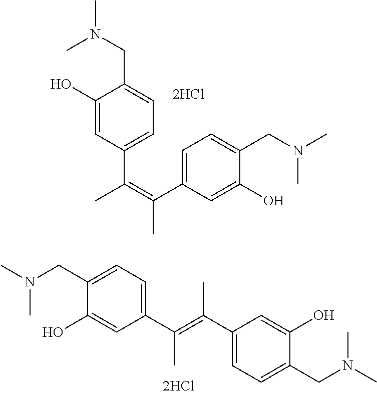

10) (Z)- and (E)-5,5′-(Ethene-1,2-diyl)bis(2-((diethylamino)methyl)phenol).2HCl (“NB-10”):

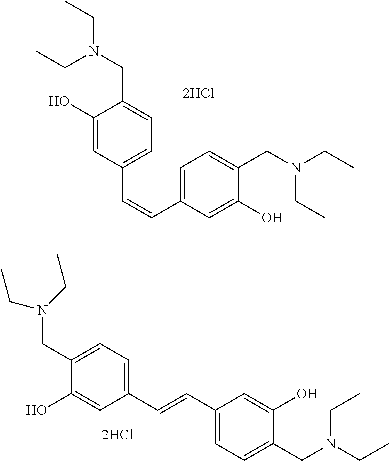

11) (Z)- and (E)-5,5′-(Ethene-1,2-diyl)bis(2-((dimethylamino)methyl)phenol).2HCl (“NB-11”):

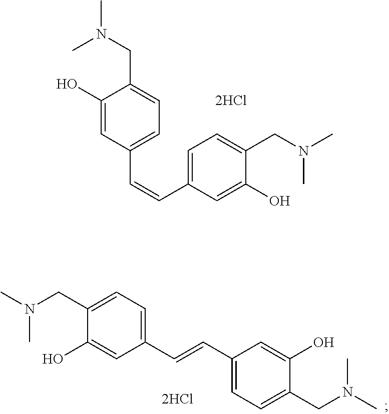

12) (Z)- and (E)-5,5′-(Ethene-1,2-diyl)bis(2-(piperidin-1-ylmethyl)phenol) (“NB-12”):

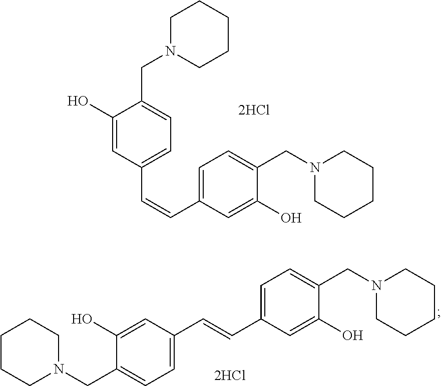

13) (Z) or (E)-4,4′-(But-2-ene-2,3-diyl)bis(2-((dimethylamino)methyl)phenol) (“NB-13”):

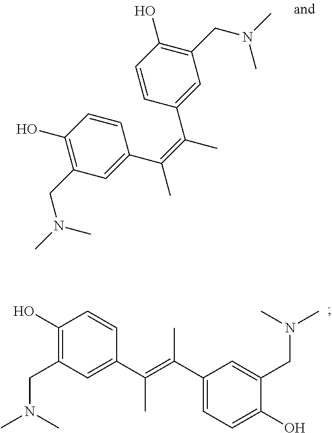

14) (Z) and (E)-5,5′-(Ethene-1,2-diyl)bis(2-(pyrrolidin-1-ylmethyl)phenol) (“NB-14”):

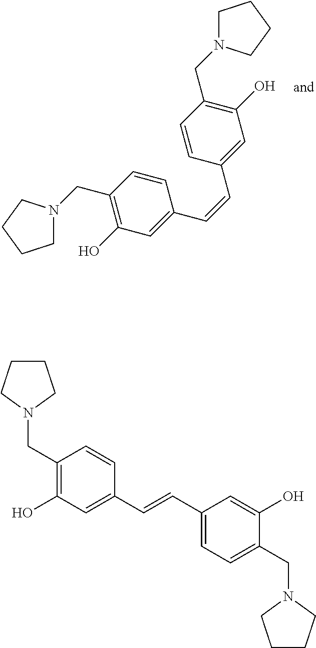

15) (Z) and (E)-4,4′-(Ethene-1,2-diyl)bis(2-((dimethylamino)methyl)phenol) (“NB-15”):

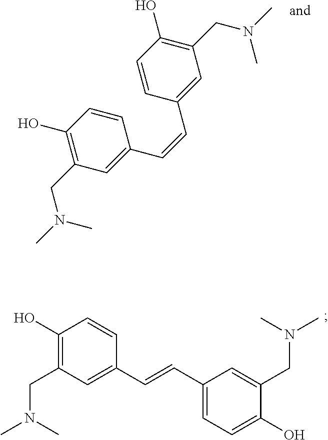

16) (Z) and (E)-5,5′-(But-2-ene-2,3-diyl)bis(2-(piperidin-1-ylmethyl)phenol) (“NB-16”):

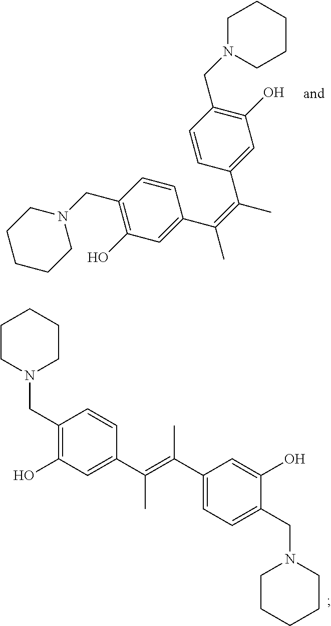

17) (Z) and (E)-(5,5′-(but-2-ene-2,3-diyl)bis(2-(pyrrolidin-1-ylmethyl)phenol) (“NB-17”):

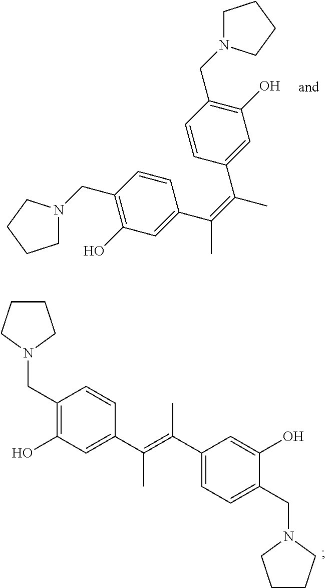

18) (Z) and (E)-5,5′-(Ethene-1,2-diyl)bis(2-(morpholinomethyl)phenol) (“NB-18”):

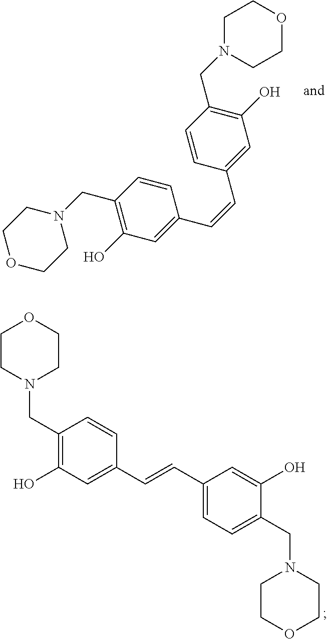

19) (Z) and (E)-5,5′-(But-2-ene-2,3-diyl)bis(2-(morpholinomethyl)phenol) (“NB-19”):

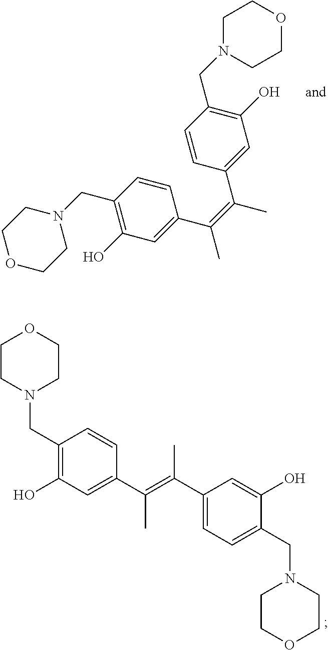

20) (Z) and (E)-4,4′-(Ethene-1,2-diyl)bis(2-(piperidin-1-ylmethyl)phenol) (“NB-20”):

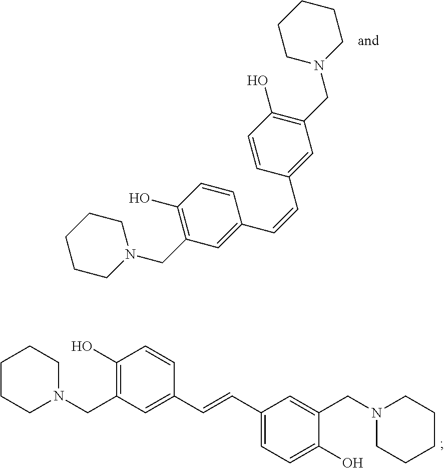

21) (Z) and (E)-4,4′-(Ethene-1,2-diyl)bis(2-(pyrrolidin-1-ylmethyl)phenol) (“NB-21”):

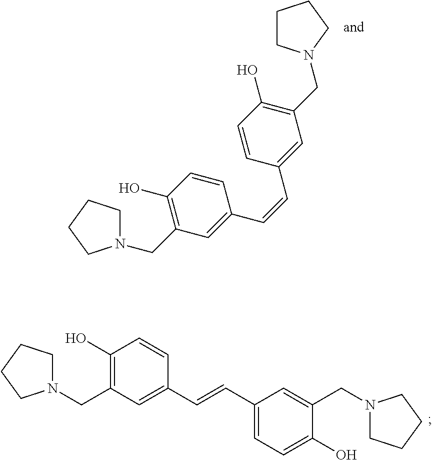

22) (Z) and (E)-4,4′-(Ethene-1,2-diyl)bis(2-(morpholinomethyl)phenol) (“NB-22”):

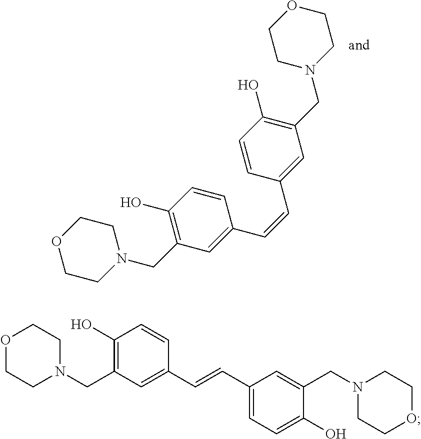

23) (Z) and (E)-4,4′-(Hex-3-ene-3,4-diyl)bis(2-((dimethylamino)methyl)phenol) (“NB-23”):

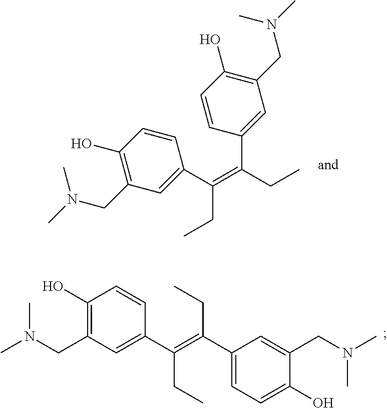

24) (Z) and (E)-4,4′-(Hex-3-ene-3,4-diyl)bis(2-(pyrrolidin-1-ylmethyl)phenol) (“NB-24”):

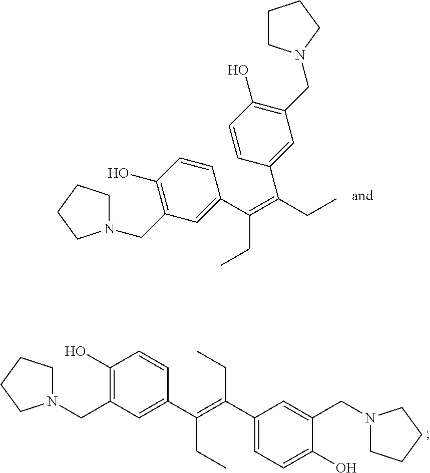

25) (Z) and (E)-4,4′-(Hex-3-ene-3,4-diyl)bis(2-(piperidin-1-ylmethyl)phenol) (“NB-25”):

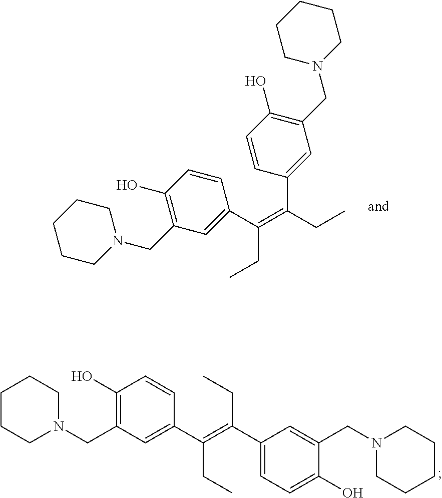

26) (Z) and (E)-4,4′-(Hex-3-ene-3,4-diyl)bis(2-(morpholinomethyl)phenol) (“NB-26”):

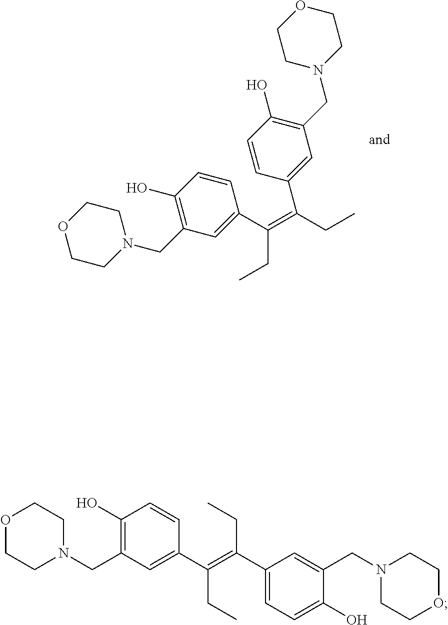

27) (Z) and (E)-4,4′-(But-2-ene-2,3-diyl)bis(2-(piperidin-1-ylmethyl)phenol) (“NB-27”):

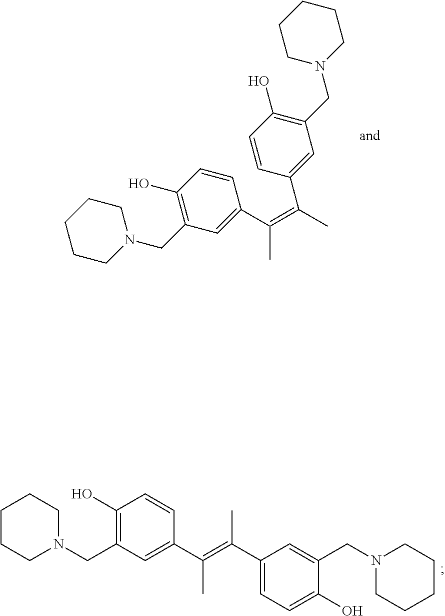

28) (Z) and (E)-4,4′-(But-2-ene-2,3-diyl)bis(2-(pyrrolidin-1-ylmethyl)phenol) (“NB-28”):

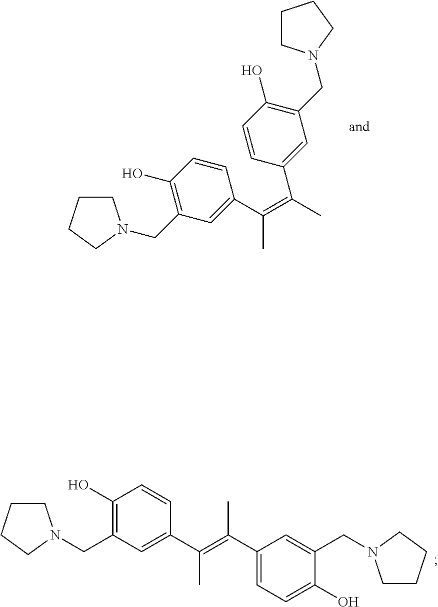

29) (Z) and (E)-2-((Diethylamino)methyl)-4-(4-(4-hydroxyphenyl)hex-3-en-3-yl)phenol (“NB-29”):

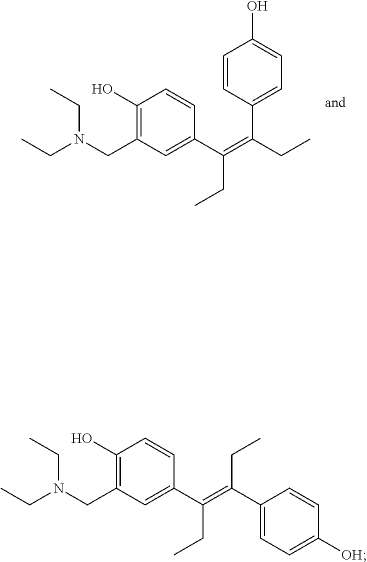

30) (Z) and (E)-4,4′-(Hex-3-ene-3,4-diyl)diphenol (“NB-30”)

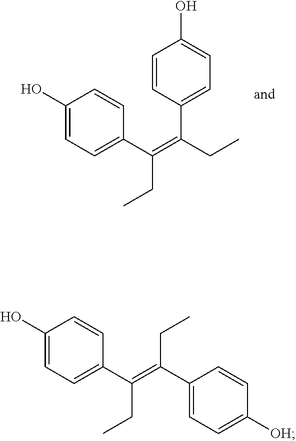

31) (Z) and (E)-4,4′-(But-2-ene-2,3-diyl)diphenol (“NB-31”):

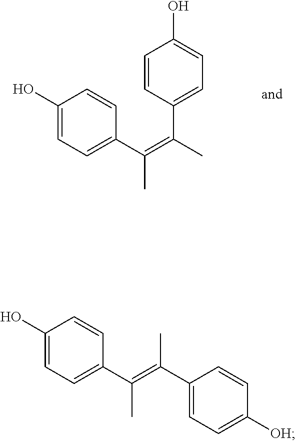

32) (Z) and (E)-3,3′-(Ethene-1,2-diyl)diphenol (“NB-32”):

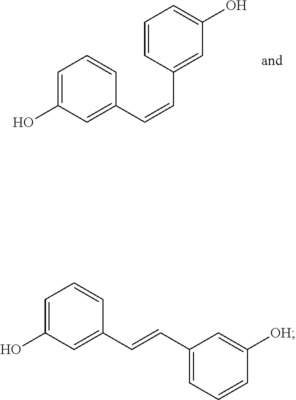

33) (Z) and (E)-3,3′-(But-2-ene-2,3-diyl)diphenol (“NB-33”):

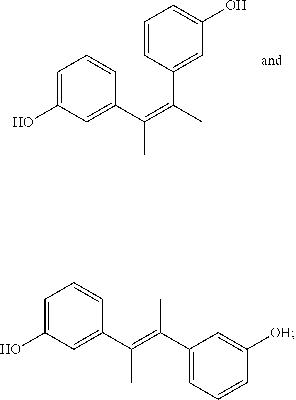

34) (Z) and (E)-4,4′-(Ethene-1,2-diyl)diphenol (“NB-34”):

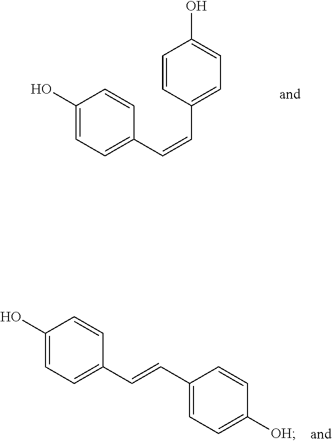

35) (Z) and (E)-4,4′-(Hex-3-ene-3,4-diyl)bis(2-((diethylamino)methyl)phenol) (“G6”):

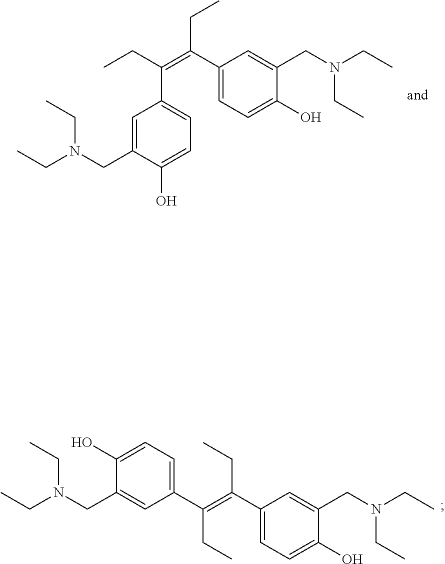

or a pharmaceutically acceptable salt, ester, hydrate or solvate thereof.

In one embodiment, the compound is selected from the group (Group (D)) consisting of NB-1, NB-2, NB-3, NB-4, NB-5, NB-6, NB-7, NB-8, NB-9, NB-10, NB-11 and NB-12, or a pharmaceutically acceptable salt, ester, hydrate or solvate thereof.

In still another embodiment, the compound is a compound of Formula (III):

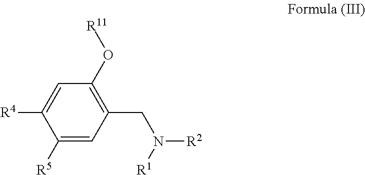

wherein
R¹ and R² are each independently H, —(C₁-C₄)alkyl, —(C₂-C₈)alkenyl, —(C₂-C₈)alkynyl,

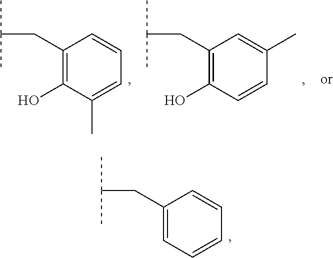

wherein —(C₁-C₄)alkyl can be further substituted with one or more hydroxy or halogen; or
R¹ and R², together with the N-atom to which they are attached, to form a 5-membered or 6-membered heterocyclic ring, provided that when R¹ and R² together with the N-atom form a piperazine ring, the second nitrogen on the piperazine ring can be further substituted with —(C₁-C₄)alkyl, —(C₃-C₇)cycloalkyl, aryl or acyl, wherein —(C₁-C₄)alkyl, —(C₃-C₇)cycloalkyl, aryl or acyl can be substituted with one or more hydroxy, halogen or —(C₁-C₃)alkyl;
R¹¹ is H, acyl, tosyl, —(C₁-C₄)alkyl, or aryl;
R⁴ and R⁵ are H or R¹², provided that one of R⁴ and R⁵ is H, and the other is R¹²;
R¹² is

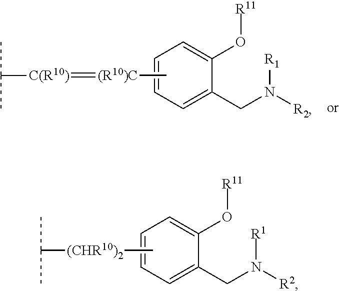

wherein the aryl group to which both R⁴ and R⁵ are attached can be meta or para to the —OR¹¹ in the aromatic ring of R¹²;
R¹⁰ is hydrogen, or —(C₁-C₃)alkyl;
or a pharmaceutically acceptable salt, ester, hydrate or solvate thereof;
provided that the compound is not:
i. 4,4′-(Hex-3-ene-3,4-diyl)bis(2-((diethylamino)methyl)phenol); or
ii. 4,4′-(Ethene-1,2-diyl)bis(2-((diethylamino)methyl)phenol); or
iii. 5,5′-(Hex-3-ene-3,4-diyl)bis(2-((dimethylamino)methyl)phenol); or
iv. 4,4′-(Hex-3-ene-3,4-diyl)bis(2-((dimethylamino)methyl)phenol); or
v. 4,4′-(Ethene-1,2-diyl)bis(2-((dimethylamino)methyl)phenol); or
vi. 4,4′-(Hex-3-ene-3,4-diyl)bis(2-((diethylamino)methyl)phenol); or
vii. 4,4′-(Ethene-1,2-diyl)bis(2-((diethylamino)methyl)phenol); or
viii. 4,4′-(Ethene-1,2-diyl)bis(2-(pyrrolidin-1-ylmethyl)phenol).

In one embodiment, R¹¹ is hydrogen in Formula (III). In another embodiment, R¹⁰ for each occurrence is hydrogen, methyl or ethyl.

In one embodiment, R¹² is

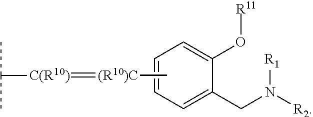

In one embodiment of the compounds of formula (III), R⁴ is R¹² and R⁵ is H. In one embodiment, the aryl group to which R⁴ and R⁵ are attached is meta to the —OR¹¹ in the aromatic ring of R¹².

In one embodiment of the compounds of formula (III), R¹⁰ for each occurrence is hydrogen or methyl. In another embodiment, R¹ and R² for each occurrence are —(C₁-C₄)alkyl. In still another embodiment, R¹ and R² together with the N-atom to which they are attached form a piperidinyl, pyrrolidinyl or imidazolyl ring, wherein R¹⁰ is the same for each occurrence.

In one embodiment, R¹⁰ is ethyl. In another embodiment, R¹ and R² are ethyl, or isopropyl. In another embodiment, R¹ and R² together with the N-atom to which they are attached form a pyrrolidinyl or imidazolyl ring.

In certain embodiments, R⁴ is H and R⁵ is R¹². In one embodiment, the aryl group to which R⁴ and R⁵ are attached is para to the —OR¹¹ in the aromatic ring of R¹².

In one embodiment, R¹⁰ is methyl. In another embodiment, R¹ and R² for each occurrence are —(C₁-C₄)alkyl, or R¹ and R² together with the N-atom to which they are attached form a 5-membered or 6-membered heterocyclic ring. In another embodiment, when R¹⁰ is H or ethyl and R¹⁰ is the same for each occurrence, R¹ and R² are propyl or isopropyl.

In another embodiment, when R¹⁰ is ethyl, R¹ and R² together with the N-atom to which they are attached form a piperidinyl, pyrrolidinyl or imidazolyl ring.

The names for the compounds herein are meant to expressly encompass both cis- and trans-isomers of each of these compounds.

In one embodiment, the compound is a stilbene or stilbenoid derivative.

In another embodiment, the compound is (Z) or (E)-4,4′-(hex-3-ene-3,4-diyl)bis(2-((diethylamino)methyl)phenol) (“G6”), or a pharmaceutically acceptable salt, ester, hydrate or solvate thereof.

Also, the compounds of the invention may contain one or more asymmetric centers and thus occur as racemates and racemic mixtures, single enantiomers, individual diastereomers and diastereomeric mixtures. All such isomeric forms of these compounds are expressly comtemplated. The compounds of the invention may also be represented in multiple tautomeric forms, in such instances, the invention expressly includes all tautomeric forms of the compounds described herein. All such isomeric forms of such compounds are expressly included. Crystal forms of the compounds described herein are also included.

The compounds of the invention are capable of modulating (e.g., inhibiting or stimulating) (directly or indirectly) Jak2-binding activity and methods using the compounds thereof. Other aspects of the compounds and methods include those wherein the subject is identified as having the Jak2-V617F mutant; wherein the subject is identified as having the K603Q, D620E or C644S mutation in the Jak2 JH2 domain; wherein the subject is identified as having the K603Q, D620E and C644S mutations in the Jak2 JH2 domain; or wherein the subject is identified as having the K603Q, D620E and C644S mutations in the Jak2 JH2 domain and is identified as not having the Jak2-V617F mutant.

The invention also relates to pharmaceutically acceptable esters, salts, solvates, hydrates or prodrugs thereof of the compounds delineated above.

Naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

The compounds of the invention can be prepared according to a variety of methods, some of which are known in the art. Methods of synthesizing the compounds of the invention are exemplified in Example 1; other methods of preparation will be apparent to one of ordinary skill in the art. Methods for optimizing reaction conditions, if necessary minimizing competing by-products, are known in the art. The methods may also additionally include steps, either before or after the steps described specifically herein, to add or remove suitable protecting groups in order to ultimately allow synthesis of the compounds herein. In addition, various synthetic steps may be performed in an alternate sequence or order to give the desired compounds. Synthetic chemistry transformations and protecting group methodologies (protection and deprotection) useful in synthesizing the applicable compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

Also, the invention provides compounds which associate with or bind to the kinase binding pocket of Jak2 defined by one or more of the following residues; GLN14 LEU15 GLY16 LYS17 GLY21 SER22 VAL39 ALA40 VAL41 ARG57 ILE70 ARG86 ILE88 MET89 GLU90 TYR91 LEU92 PRO93 TYR94 GLY95 LEU97 ARG98 ALA138 THR139 ARG140 ILE152 GLY153 ASP154 PHE155, or a Jak2 protein-protein binding partner binding pocket (including targets where Jak2 mediates a biological process or mechanism) that are useful in the methods described herein. In one aspect, the interaction of the test compound and the Jak2 kinase domain comprises one H-bond acceptor interaction with Glu90 and one H-bond donor interaction with Leu92. Without wishing to be bound by any theory, it appears that these interactions may be important in contributing to activity of certain potent Jak2 inhibitors.

### 3. USES OF THE COMPOUNDS OF THE INVENTION

Somatic mutations in the Jak2 allele are described in virtually all patients diagnosed with polycythemia vera (PV), and about 50% of patients with essential thrombocythemia (ET) and chronic idiopathic myelofibrosis (CIMF) (Kaushansky, K. Best Pract Res. Clin. Haematol. 2007, 20:5-12). The most common Jak2 mutation is the result of a G→T point mutation at nucleotide 1849 within exon 12, resulting in a phenylalanine substation for valine at codon 617 (V617F). The mutation is located in the JAK homology 2 (JH2) negative regulatory domain and its presence results in increased Jak2 kinase activity that is unresponsive to the negative feedback mechanisms that govern normal cell growth. A causal role for the mutation is supported in vivo by murine transfection studies resulting in erythrocytosis and myelofibrosis in recipient animals (Lacout C. et al. Blood 2006, 108: 1652-1660). Additional somatic, Jak2 gain-of-function mutations have been detected in exon 12 in patients with V617F negative erythrocytosis (Zhang S J, Int J. Lab. Hematol. 2007, 29:71-72) (See also PCT Patent Application No.: PCT/US08/007,073, the contents of which are incorporated herein by reference).

The present inventors have now discovered a class of small molecules that are novel Jak2 tyrosine kinase inhibitors. In particular, in certain embodiments, a Jak2 small molecule inhibitor is a compound of Formula (II) as above defined, or its pharmaceutically acceptable salt, ester, hydrate or solvate thereof. In one embodiments, the In certain embodiments, the Jak2 small molecule inhibitor is a compound of Formula (I). In certain embodiments, the inhibitor is a compound of Formula (III). In certain embodiments, the Jak2 small molecule inhibitor is a compound of Group (A), (B), (C) or (D) as above defined, its pharmaceutically acceptable salt, hydrate or solvate thereof. In one embodiment, the compound is a compound of Group (B), or a pharmaceutically acceptable salt, ester, hydrate or solvate thereof. In another embodiment, the compound is a compound selected from Group (C), or a pharmaceutically acceptable salt, ester, hydrate or solvate thereof. In still another embodiment, the compound is a compound selected from Group (D), or a pharmaceutically acceptable salt, ester, hydrate or solvate thereof.

In yet another embodiment, the compound is a stilbene or stilbenoid derivative. In one embodiment, the compound is (E) or (Z)-4,4′-(hex-3-ene-3,4-diyl)bis(2-((diethylamino)methyl)phenol), or a pharmaceutically acceptable salt, ester, hydrate or solvate thereof.

In another aspect, the invention provides methods for treating a subject for a Jak2-mediated disease or disorder (e.g., polycythemia vera, essential thrombocythemia, angiogenic myeloid metaplasia), by administering to the subject an effective amount of a compound of the invention. In one embodiment, the compound of the invention is a compound of Formula (II). In another embodiment, the compound administered to the subject is a compound of Formula (I) or (III).

In certain embodiments, the compound is selected from Group (A), (B), (C) or (D) as above described, or its pharmaceutically acceptable salt, ester, hydrate or solvate thereof. In one embodiment, the compound is a compound selected from Group (B), or a pharmaceutically acceptable salt, ester, hydrate or solvate thereof. In another embodiment, the compound is a compound selected from Group (C), or a pharmaceutically acceptable salt, ester, hydrate or solvate thereof. In still another embodiment, the compound is a compound selected from Group (D), or a pharmaceutically acceptable salt, ester, hydrate or solvate thereof.

In yet another embodiment, the compound is a stilbene or stilbenoid derivative. In another embodiment, the compound is (E) or (Z)-4,4′-(hex-3-ene-3,4-diyl)bis(2-((diethylamino)methyl)phenol), or a pharmaceutically acceptable salt, ester, hydrate or solvate thereof.

In certain embodiments, the compound of the invention is administered to a subject at a dose between about 0.001 mg/Kg/day and about 200 mg/Kg/day. In another embodiment, the compound of the invention is administered to the subject at a dose between about 0.1 mg/Kg/day and about 10 mg/Kg/day. In one embodiment, the compound of the invention is administered to the subject at a dose about 1 mg/Kg/day.

The compounds of the invention may either directly or indirectly modulate having Jak2 or Jak2 mutant activity can be contacted with a compound of the invention to inhibit disease or disorder processes or modulation of the Jak2 metabolic cascade. Contacting cells or administering the compounds of the invention to a subject is one method of treating a cell or a subject suffering from or susceptible to unwanted or undesired Jak2 or a Jak2 mutant mediated disorder.

In one embodiment, the compounds of the invention may either directly or indirectly modulate having Jak2 or Jak2 mutant activity by inhibiting Jak2 autophosphorylation. In another embodiment, the compounds of the invention do not inhibit c-Src or Tyk2 autophosphorylation as effectively as Jak2 autophosphorylation. In aspects, the compounds demonstrate a level of Jak2 (or Jak2 mutant) autophosphorylation inhibition that is at least 2-, 5-, 10-, 25-, 50- or 100-fold higher than c-Src or Tyk2 autophosphorylation inhibition.

In certain embodiments, the methods of the invention include administering to a subject a therapeutically effective amount of a compound of the invention in combination with another pharmaceutically active compound. In certain embodiments, such an effective amount is at a dose between about 0.001 mg/Kg/day and about 200 mg/Kg/day, between about 0.001 mg/Kg/day and about 30 mg/Kg/day. In another embodiment, the compound of the invention is administered to the subject at a dose between about 0.1 mg/Kg/day and about 10 mg/Kg/day. In one embodiment, the compound of the invention is administered to the subject at a dose about 1 mg/Kg/day.

Examples of pharmaceutically active compounds include compounds known to treat proliferative disorders, e.g., anticancer agents, antitumor agents, antiangiogenesis agents, chemotherapeutics, antibodies, etc. Other pharmaceutically active compounds that may be used can be found in Harrison's Principles of Internal Medicine, Thirteenth Edition, Eds. T. R. Harrison et al. McGraw-Hill N.Y., NY; and the Physicians Desk Reference 50th Edition 1997, Oradell N.J., Medical Economics Co., the complete contents of which are expressly incorporated herein by reference. The compound of the invention and the pharmaceutically active compound may be administered to the subject in the same pharmaceutical composition or in different pharmaceutical compositions (at the same time or at different times).

In certain embodiments, the compound of the invention can be used in combination therapy with an existing anti-cancer therapeutics. Conventional treatment regimens include, for example, radiation, drugs, or a combination of both. In addition to radiation, the following drugs, usually in combinations with each other, are often used to treat acute leukemias: vincristine, prednisone, methotrexate, mercaptopurine, cyclophosphamide, and cytarabine. Other examples include, for example, doxorubicin, cisplatin, taxol, 5-fluorouracil, etoposid, etc., which demonstrate advantages (e.g., chemosensitization of cells) in combination with the compounds described herein. In chronic leukemia, for example, busulfan, melphalan, and chlorambucil can be used in combination. Proteosome inhibitors (e.g., MG-132), hydroxyureas (e.g., Hydrea or hydroxycarbamide) or kinase inhibitors (e.g., GLEEVEC) can also be used in combination with the compounds herein. Most conventional anti-cancer drugs are highly toxic and tend to make patients quite ill while undergoing treatment. Vigorous therapy is based on the premise that unless every cancerous cell is destroyed, the residual cells will multiply and cause a relapse.

Determination of a therapeutically effective anti-proliferative amount or a prophylactically effective anti-proliferative amount of the compound of the invention, can be readily made by the physician or veterinarian (the “attending clinician”), as one skilled in the art, by the use of known techniques and by observing results obtained under analogous circumstances. The dosages may be varied depending upon the requirements of the patient in the judgment of the attending clinician; the severity of the condition being treated and the particular compound being employed. In determining the therapeutically effective anti-proliferative amount or dose, and the prophylactically effective anti-proliferative amount or dose, a number of factors are considered by the attending clinician, including, but not limited to: the specific disorder involved; pharmacodynamic characteristics of the particular agent and its mode and route of administration; the desired time course of treatment; the species of mammal; its size, age, and general health; the specific disease involved; the degree of or involvement or the severity of the disease; the response of the individual patient; the particular compound administered; the mode of administration; the bioavailability characteristics of the preparation administered; the dose regimen selected; the kind of concurrent treatment (i.e., the interaction of the compound of the invention with other co-administered therapeutics); and other relevant circumstances.

Treatment can be initiated with smaller dosages, which are less than the optimum dose of the compound. Thereafter, the dosage may be increased by small increments until the optimum effect under the circumstances is reached. For convenience, the total daily dosage may be divided and administered in portions during the day if desired. A therapeutically effective amount and a prophylactically effective anti-proliferative amount of a compound of the invention is expected to vary from about 0.1 milligram per kilogram of body weight per day (mg/kg/day) to about 200 mg/kg/day. In certain embodiments, such a dosage is between about 0.001 mg/Kg/day and about 30 mg/Kg/day. In another embodiment, the dosage is between about 0.1 mg/Kg/day and about 10 mg/Kg/day. In one particular embodiment, the dosage is about 1 mg/Kg/day.

Compounds of the invention used in the prevention or treatment of disease or disorders in animals, e.g., dogs, chickens, and rodents, may also be useful in treatment of tumors in humans. Those skilled in the art of treating tumors in humans will know, based upon the data obtained in animal studies, the dosage and route of administration of the compound to humans.

In yet another aspect, the invention provides the use of a compound of any of the formulae herein, alone or together with one or more additional therapeutic agents in the manufacture of a medicament, either as a single composition or as separate dosage forms, for treatment or prevention in a subject of a disease, disorder or symptom set forth herein. Another aspect of the invention is a compound of the formulae herein for use in the treatment or prevention in a subject of a disease, disorder or symptom thereof delineated herein.

The identification of those patients who are in need of prophylactic treatment for Jak2-mediated disorders is well within the ability and knowledge of one skilled in the art. Certain of the methods for identification of patients which are at risk of developing Jak2-mediated disorders which can be treated by the subject method are appreciated in the medical arts, such as family history, and the presence of risk factors associated with the development of that disease state in the subject patient. A clinician skilled in the art can readily identify such candidate patients, by the use of, for example, clinical tests, physical examination and medical/family history.

A method of assessing the efficacy of a treatment in a subject includes determining the pre-treatment extent of a Jak2-mediated disorder by methods well known in the art (e.g., determining tumor size or screening for cancer markers where the Jak2-mediated disorder is present) and then administering a therapeutically effective amount of an inhibitor of cell proliferation (e.g., those described herein) according to the invention to the subject. After an appropriate period of time after the administration of the compound (e.g., 1 day, 1 week, 2 weeks, one month, six months), the extent of the Jak2-mediated disorder is determined again. Certain embodiments provide that the determination takes place within 24 to 72 hours of the administration. One embodiment provides that the determination takes place within 48 hours of the administration.

The modulation (e.g., decrease) of the extent or invasiveness of the Jak2-mediated disorder indicates efficacy of the treatment. The extent or invasiveness of the Jak2-mediated disorder may be determined periodically throughout treatment. For example, the extent or invasiveness of the Jak2-mediated disorder may be checked every few hours, days or weeks to assess the further efficacy of the treatment. A decrease in extent or invasiveness of the Jak2-mediated disorder indicates that the treatment is efficacious. The method described may be used to screen or select patients that may benefit from treatment with an inhibitor of a Jak2-mediated disorder.

As used herein, “obtaining a biological sample from a subject,” includes obtaining a sample for use in the methods described herein. A biological sample is described above.

Yet another aspect presents a method to identify a compound that modulates the interaction of Jak2-mediated binding partner, or specific domains thereof. The method may include obtaining the crystal structure of a Jak2-mediated binding partner, or specific domains thereof (optionally apo form or complexed) or obtaining the information relating to the crystal structure of a Jak2-mediated binding partner, or specific domains thereof (optionally apo form or complexed), in the presence and/or absence of the test compound. Compounds may then be computer modeled into or on the Jak2-mediated binding partner, or specific domains thereof binding site of the crystal structure to predict stabilization of the interaction between the Jak2-mediated binding partner, or specific domains thereof and the test compound. Once potential modulating compounds are identified, the compounds may be screened using cellular assays, such as the ones identified herein and competition assays known in the art (see also PCT Publication WO2008/153900, the contents of which are incorporated herein by reference). Compounds identified in this manner are useful as therapeutic agents.

In another aspect, a compound of the formulae herein is packaged in a therapeutically effective amount with a pharmaceutically acceptable carrier or diluent. The composition may be formulated for treating a subject suffering from or susceptible to a Jak2-mediated disorder, and packaged with instructions to treat a subject suffering from or susceptible to a Jak2-mediated disorder.

In another aspect, the invention provides methods for inhibiting cell proliferation. In one embodiment, a method of inhibiting cell proliferation (or a Jak2-mediated disorder) according to the invention includes contacting cells with a compound capable of modulating Jak2 or a Jak2-mediated binding partner, or specific domains thereof. In either embodiment, the contacting may be in vitro, e.g., by addition of the compound to a fluid surrounding the cells, for example, to the growth media in which the cells are living or existing. The contacting may also be by directly contacting the compound to the cells. Alternately, the contacting may be in vivo, e.g., by passage of the compound through a subject; for example, after administration, depending on the route of administration, the compound may travel through the digestive tract or the blood stream or may be applied or administered directly to cells in need of treatment.

In certain embodiments, the methods includes contacting cells with compounds of the invention for from 24 to 72 hours. In another embodiment, the methods includes contacting cells with compounds of the invention up to 48 hours.

In certain embodiments, a method of inhibiting a Jak2-mediated disorder in a subject includes administering an effective amount of a compound of the invention (i.e., a compound described herein) to the subject. The administration may be by any route of administration known in the pharmaceutical arts. The subject may have a Jak2-mediated disorder, may be at risk of developing a Jak2-mediated disorder, or may need prophylactic treatment prior to anticipated or unanticipated exposure to a conditions capable of increasing susceptibility to a Jak2-mediated disorder, e.g., exposure to carcinogens or to ionizing radiation.

The subject may be at risk of a Jak2-mediated disorder, may be exhibiting symptoms of a Jak2-mediated disorder, may be susceptible to a Jak2-mediated disorder and/or may have been diagnosed with a Jak2-mediated disorder.

If the modulation of the status indicates that the subject may have a favorable clinical response to the treatment, the subject may be treated with the compound. For example, the subject can be administered therapeutically effective dose or doses of the compound.

The methods can be performed on cells in culture, e.g. in vitro or ex vivo, or on cells present in an animal subject, e.g., in vivo. Compounds of the invention can be initially tested in vitro using primary cultures of proliferating cells, e.g., transformed cells, tumor cell lines, and the like.

In another aspect, the methods herein include those: wherein a compound of the invention is administered to a subject for treating or preventing Jak2 mediated disease or disorder; or wherein a compound of the invention is administered to a subject to reduce Jak2-dependent cell growth; wherein a compound of the invention is administered to a subject for treating a hematological disease or disorder; wherein a compound of the invention is adminstered to a subject for treating cancer.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method). In other methods, the subject is pre-screened or identified as in need of such treatment by assessment for a relevant marker or indicator of suitability for such treatment.

The methods can be performed on cells in culture, e.g. in vitro or ex vivo, or on cells present in an animal subject, e.g., in vivo. Compounds of the invention can be initially tested in vitro using cells or other mammalian or non-mammalian animal models. Alternatively, the effects of a compound of the invention can be characterized in vivo using animals models.

### 4. PHARMACEUTICAL COMPOSITIONS

The invention also provides a pharmaceutical composition, comprising an effective amount of a compound of the invention and a pharmaceutically acceptable carrier. In a further embodiment, the effective amount is effective to treat a Jak2-mediated disease or disorder, as described previously.

In an embodiment, the compound of the invention is administered to the subject using a pharmaceutically-acceptable formulation, e.g., a pharmaceutically-acceptable formulation that provides sustained delivery of the compound of the invention to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject. In other embodiments, as described in detail below, the pharmaceutical compositions of the invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” refers to those compound of the invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier is “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a compound of the invention include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, e.g., from about 5 percent to about 70 percent, e.g., from about 10 percent to about 30 percent.

Methods of preparing these compositions include the step of bringing into association a compound of the invention with the carrier and, optionally, one or more accessory ingredients. In general, the formulations are prepared by uniformly and intimately bringing into association a compound of the invention with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the invention as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compound of the invention include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to the active compound of the invention may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the invention which are suitable for vaginal administration also include pessaries, tampons, creams, gels, pastes, foams or spray formulations containing such carriers as are known in the art to be appropriate.

Dosage forms for the topical or transdermal administration of a compound of the invention include powders, sprays, ointments, pastes, creams, lotions, gels, solutions, patches and inhalants. The active compound of the invention may be mixed under sterile conditions with a pharmaceutically-acceptable carrier, and with any preservatives, buffers, or propellants which may be required.

The ointments, pastes, creams and gels may contain, in addition to a compound of the invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of the invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The compound of the invention can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a compound of the invention to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Ophthalmic formulations, eye ointments, powders, solutions and the like, are also contemplated as being within the scope of the invention.

Pharmaceutical compositions of the invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers, which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of compound of the invention in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compound of the invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, the compound of the invention, which may be used in a suitable hydrated form, and/or the pharmaceutical compositions of the invention are formulated into pharmaceutically-acceptable dosage forms by conventional methods known to those of skill in the art.

Actual dosage levels and time course of administration of the active ingredients in the pharmaceutical compositions of the invention may be varied so as to obtain an amount of the active ingredient which is effective to achieve the desired therapeutic response for a particular patient, composition, and mode of administration, without being toxic to the patient. In certain embodiments, the time course of administration of the active ingredients is from 24 to 72 hours. In one embodiment, the time course of administration is up to 48 hours.

A preferred dose of the compound of the invention is the maximum that a patient can tolerate and not develop serious side effects. For example, the compound of the invention is administered at a concentration of about 0.001 mg to about 200 mg per kilogram of body weight, about 0.001-about 30 mg/kg or about 0.1 mg-about 10 mg/kg of body weight. Ranges intermediate to the above-recited values are also intended to be part of the invention. A particular example is that a compound of the invention is administered at a dose about 1 mg/Kg/day.

### 6. SCREENING METHODS AND SYSTEMS

In another aspect, the invention provides a machine readable storage medium which comprises the structural coordinates of either one or both of the binding pockets identified herein, or similarly shaped, homologous binding pockets. Such storage medium encoded with these data are capable of displaying a three-dimensional graphical representation of a molecule or molecular complex which comprises such binding pockets on a computer screen or similar viewing device.

The invention also provides methods for designing, evaluating and identifying compounds which bind to the aforementioned binding pockets. Thus, the computer produces a three-dimensional graphical structure of a molecule or a molecular complex which comprises a binding pocket.

In another embodiment, the invention provides a computer for producing a three-dimensional representation of a molecule or molecular complex defined by structure coordinates of Jak2 or domains thereof, or a three-dimensional representation of a homologue of the molecule or molecular complex, wherein the homologue comprises a binding pocket that has a root mean square deviation from the backbone atoms of the amino acids of not more than 2.0 (more preferably not more than 1.5) angstroms.

In exemplary embodiments, the computer or computer system can include components which are conventional in the art, e.g., as disclosed in U.S. Pat. No. 5,978,740 and/or 6,183,121 (incorporated herein by reference). For example, a computer system can includes a computer comprising a central processing unit (“CPU”), a working memory (which may be, e.g., RAM (random-access memory) or “core” memory), a mass storage memory (such as one or more disk drives or CD-ROM drives), one or more cathode-ray tube (CRT) or liquid crystal display (LCD) display terminals, one or more keyboards, one or more input lines, and one or more output lines, all of which are interconnected by a conventional system bus.

Machine-readable data of the invention may be inputted to the computer via the use of a modem or modems connected by a data line. Alternatively or additionally, the input hardware may include CD-ROM drives, disk drives or flash memory. In conjunction with a display terminal, a keyboard may also be used as an input device.

Output hardware coupled to the computer by output lines may similarly be implemented by conventional devices. By way of example, output hardware may include a CRT or LCD display terminal for displaying a graphical representation of a binding pocket of the invention using a program such as QUANTA or PYMOL. Output hardware might also include a printer, or a disk drive to store system output for later use.

In operation, the CPU coordinates the use of the various input and output devices, coordinates data accesses from the mass storage and accesses to and from working memory, and determines the sequence of data processing steps. A number of programs may be used to process the machine-readable data of the invention, including commercially-available software.

A magnetic storage medium for storing machine-readable data according to the invention can be conventional. A magnetic data storage medium can be encoded with a machine-readable data that can be carried out by a system such as the computer system described above. The medium can be a conventional floppy diskette or hard disk, having a suitable substrate which may be conventional, and a suitable coating, which may also be conventional, on one or both sides, containing magnetic domains whose polarity or orientation can be altered magnetically. The medium may also have an opening (not shown) for receiving the spindle of a disk drive or other data storage device.

The magnetic domains of the medium are polarized or oriented so as to encode in manner which may be conventional, machine readable data such as that described herein, for execution by a system such as the computer system described herein.

An optically-readable data storage medium also can be encoded with machine-readable data, or a set of instructions, which can be carried out by a computer system. The medium can be a conventional compact disk read only memory (CD-ROM) or a rewritable medium such as a magneto-optical disk which is optically readable and magneto-optically writable.

In the case of CD-ROM, as is well known, a disk coating is reflective and is impressed with a plurality of pits to encode the machine-readable data. The arrangement of pits is read by reflecting laser light off the surface of the coating. A protective coating, which preferably is substantially transparent, is provided on top of the reflective coating.

In the case of a magneto-optical disk, as is well known, a data-recording coating has no pits, but has a plurality of magnetic domains whose polarity or orientation can be changed magnetically when heated above a certain temperature, as by a laser. The orientation of the domains can be read by measuring the polarization of laser light reflected from the coating. The arrangement of the domains encodes the data as described above.

Structure data, when used in conjunction with a computer programmed with software to translate those coordinates into the 3-dimensional structure of a molecule or molecular complex comprising a binding pocket may be used for a variety of purposes, such as drug discovery.

For example, the structure encoded by the data may be computationally evaluated for its ability to associate with chemical entities. Chemical entities that associate with a binding pocket of a Jak2, or specific domains thereof, and are potential drug candidates. Alternatively, the structure encoded by the data may be displayed in a graphical three-dimensional representation on a computer screen. This allows visual inspection of the structure, as well as visual inspection of the structure's association with chemical entities.

Thus, according to another embodiment, the invention relates to a method for evaluating the potential of a chemical entity to associate with a) a molecule or molecular complex comprising a binding pocket of Jak2, or specific domains thereof, or b) a homologue of the molecule or molecular complex, wherein the homologue comprises a binding pocket that has a root mean square deviation from the backbone atoms of the amino acids of not more than 2.0 (more preferably 1.5) angstroms.

This method comprises the steps of:

i) employing computational means to perform a fitting operation between the chemical entity and a binding pocket of the molecule or molecular complex; and

ii) analyzing the results of the fitting operation to quantify the association between the chemical entity and the binding pocket. The term “chemical entity”, as used herein, refers to chemical compounds, complexes of at least two chemical compounds, and fragments of such compounds or complexes.

The design of compounds that bind to or inhibit Jak2, or specific domains thereof binding pockets according to the invention generally involves consideration of several factors. First, the entity must be capable of physically and structurally associating with parts or all of the Jak2 binding site, or specific domains thereof-related binding pockets. Non-covalent molecular interactions important in this association include hydrogen bonding, van der Waals interactions, hydrophobic interactions and electrostatic interactions. Second, the entity must be able to assume a conformation that allows it to associate with the Jak2, or specific domains thereof-related binding pocket(s) directly. Although certain portions of the entity will not directly participate in these associations, those portions of the entity may still influence the overall conformation of the molecule. This, in turn, may have a significant impact on potency. Such conformational requirements include the overall three-dimensional structure and orientation of the chemical entity in relation to all or a portion of the binding pocket, or the spacing between functional groups of an entity comprising several chemical entities that directly interact with the binding pocket or homologues thereof.

The potential inhibitory or binding effect of a chemical entity on a Jak2, or specific domains thereof-related binding pocket may be analyzed prior to its actual synthesis and testing by the use of computer modeling techniques. If the theoretical structure of the given entity suggests insufficient interaction and association between it and the target binding pocket, testing of the entity is obviated. However, if computer modeling indicates a strong interaction, the molecule may then be synthesized and tested for its ability to bind to a binding pocket. This may be achieved, e.g., by testing the ability of the molecule to inhibit Jak2, or specific domains thereof activity, e.g., using assays described herein or known in the art. In this manner, synthesis of inoperative compounds may be avoided.

A potential inhibitor of a Jak2, or specific domains thereof-related binding pocket may be computationally evaluated by means of a series of steps in which chemical entities or fragments are screened and selected for their ability to associate with the Jak2, or specific domains thereof-related binding pockets.

One skilled in the art may use one of several methods to screen chemical entities or fragments for their ability to associate with Jak2, or specific domains thereof-related binding pocket. This process may begin by visual inspection of, for example, a Jak2, or specific domains thereof-related binding pocket on the computer screen based on the Jak2 binding site, or specific domains thereof structure coordinates described herein, or other coordinates which define a similar shape generated from the machine-readable storage medium. Selected fragments or chemical entities may then be positioned in a variety of orientations, or docked, within that binding pocket as defined supra. Docking may be accomplished using software such as Quanta and DOCK, followed by energy minimization and molecular dynamics with standard molecular mechanics force fields, such as CHARMM and AMBER.

Specialized computer programs (e.g., as known in the art and/or commercially available and/or as described herein) may also assist in the process of selecting fragments or chemical entities.

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates of the target binding pocket.

Instead of proceeding to build an inhibitor of a binding pocket in a step-wise fashion one fragment or chemical entity at a time as described above, inhibitory or other binding compounds may be designed as a whole or “de novo” using either an empty binding site or optionally including some portion(s) of a known inhibitor(s). There are many de novo ligand design methods known in the art, some of which are commercially available (e.g., LeapFrog, available from Tripos Associates, St. Louis, Mo.).

Other molecular modeling techniques may also be employed in accordance with the invention [see, e.g., N. C. Cohen et al., “Molecular Modeling Software and Methods for Medicinal Chemistry, J. Med. Chem., 33, pp. 883-894 (1990); see also, M. A. Navia and M. A. Murcko, “The Use of Structural Information in Drug Design”, Current Opinions in Structural Biology, 2, pp. 202-210 (1992); L. M. Balbes et al., “A Perspective of Modern Methods in Computer-Aided Drug Design”, in Reviews in Computational Chemistry, Vol. 5, K. B. Lipkowitz and D. B. Boyd, Eds., VCH, New York, pp. 337-380 (1994); see also, W. C. Guida, “Software For Structure-Based Drug Design”, Curr. Opin. Struct. Biology, 4, pp. 777-781 (1994)].

Once a compound has been designed or selected, the efficiency with which that entity may bind to a binding pocket may be tested and optimized by computational evaluation.

Specific computer software is available in the art to evaluate compound deformation energy and electrostatic interactions. Examples of programs designed for such uses include: AMBER; QUANTA/CHARMM (Accelrys, Inc., Madison, Wis.) and the like. These programs may be implemented, for instance, using a commercially-available graphics workstation. Other hardware systems and software packages will be known to those skilled in the art.

Another technique involves the in silico screening of virtual libraries of compounds, e.g., as described herein. Many thousands of compounds can be rapidly screened and the best virtual compounds can be selected for further screening (e.g., by synthesis and in vitro testing). Small molecule databases can be screened for Jak2 domain, or specific domains thereof binding pocket. In this screening, the quality of fit of such entities to the binding site may be judged either by shape complementarity or by estimated interaction energy.

### 7. KITS

The invention also features kits. Included in the kits are compounds that are capable of modulating Jak2 activity. Any compound, or one or more compounds, of the invention can be included in the kits of the invention. In one aspect, the kit includes a compound of Formula (II) as above defined, or a pharmaceutical formulation thereof. In certain embodiments, the kit includes a compound of Formula (I) or (III) as above defined, or a pharmaceutical formulation thereof. In one embodiment, the kit includes a compound of Group (A), (B), (C), or (D) as above defined, or a pharmaceutical formulation thereof. In one embodiment, the kit includes a compound that is a stilbene or stilbenoid derivative.

In another embodiment, the kit includes a compound of Group (B) as above-defined, or a pharmaceutical salt, ester, solvate or prodrug thereof. In another embodiment, the kit includes a compound of Group (D) as above-defined, or a pharmaceutical salt, ester, solvate or prodrug thereof. In still another embodiment, the kit includes compound G6 as above-defined, or a pharmaceutical salt, ester, solvate or prodrug thereof.

In certain embodiments, the kit includes a compound of the invention at a dosage of between about 0.001 mg/Kg/day and about 200 mg/Kg/day, or between about 0.001 mg/Kg/day and about 30 mg/Kg/day. In some embodiments, the kit includes the compound of the invention at a dosage of between about 0.1 mg/Kg/day and about 10 mg/Kg/day. A particular example is that the compound of the invention is included in the kit at a dosage of about 1 mg/Kg/day.

The kits also include instructions for use in treating cancer, for use in treating a hematological disorder, for use in treating a cardiac disorder, and for use in reducing Jak2-dependent cell growth.

Carrier means are suited for containing one or more container means such as vials, tubes, and the like, each of the container means comprising one of the separate elements to be used in the method. In view of the description provided herein, those of skill in the art can readily determine the apportionment of the necessary reagents among the container means.

The following examples are offered by way of illustration, not by way of limitation. While specific examples have been provided, the above description is illustrative and not restrictive. Any one or more of the features of the previously described embodiments can be combined in any manner with one or more features of any other embodiments in the invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

### EXAMPLES

### Example 1

### Synthesis of Compounds

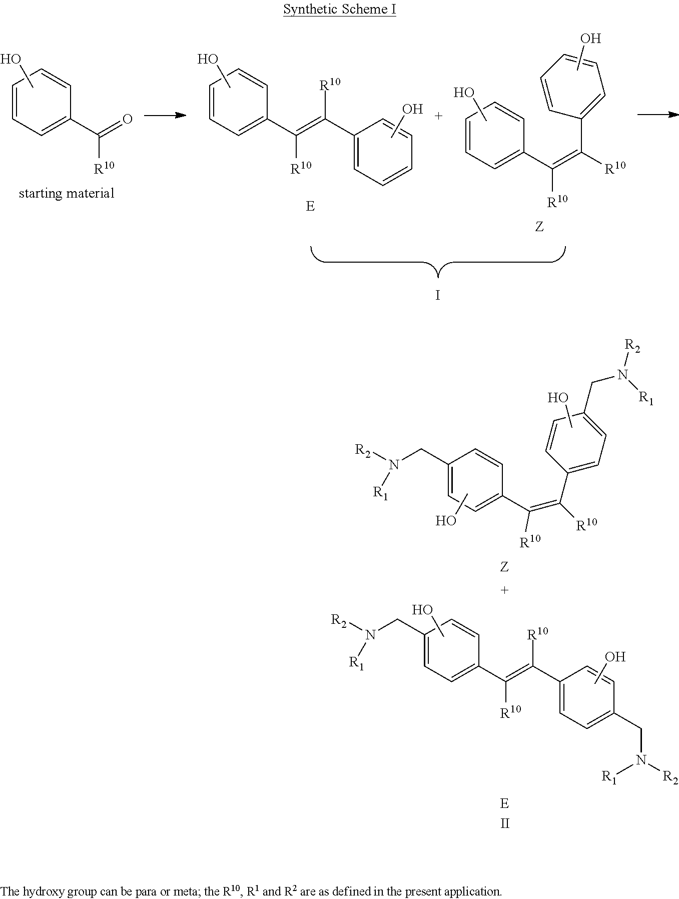

Certain compounds of the invention can be prepared by the exemplary synthetic scheme shown in Synthetic Scheme I, above.

Synthetic procedures to obtain Intermediate (I): Dry THF (180 mL) and Zinc (8 equivalents) were added into a flame dried 2 neck round bottom flask fitted with magnetic stirrer bar and reflux condensor. TiCl₄ (4 equivalents) was added dropwise at 0° C. After addition of TiCl₄ was complete, the reaction mixture was refluxed for 2 hours. The resulting brown color mixture was then cooled to 0° C. and the starting material (aldehyde or ketone) (1 equivalent), as a solution in 20 mL of dry THF, was then added slowly. The reaction mixture was refluxed and the progress of the reaction was monitored by TLC (2:3 mixture of ethyl acetate/hexane). Upon completion, reaction mixture was concentrated and diluted with ethylacetate (150 mL). To the solution in ethyl acetate, saturated K₂CO₃ solution (100 mL) was added and allowed to stir for 7 hours and filtered. The filtrate was extracted with ethyl acetate and the organic layer was washed with saturate NaCl solution, water, and dried over anhydrous Na₂SO₄. The concentrated crude mixture was column chromatographed over silica gel with 1:9 mixture of ethyl acetate:hexane to receive the E and Z isomers of Intermediate (I) as stilbene products. The stilbene products (Intermediate (I)) were dried in vacuo and characterized by ¹H— and ¹³C NMR spectroscopy.

Synthetic procedures to obtain Product (II): Intermediate (I) (1 equivalent) was dissolved in 15 mL of methanol in a one neck round bottom flask, paraformaldehyde (2.1 equivalents) and appropriate amine (2.2 equivalents) was added. The reaction mixture was allowed to reflux and the progress of the reaction was monitored using TLC (2:3 mixture of ethylacetate:hexane). Upon completion the reaction mixture was cooled to room temperature and concentrated in vacuo. The residue was dissolved in ethylacetate and treated with 1M HCl solution. Aqueous phase is separated, treated with 1M NaOH solution until pH is 7, and extracted with ethylacetate. Organic layer was washed with saturated NaCl solution, water, dried over anhydrous Na₂SO₄ and concentrated in vacuo to obtain Product (II) as a mixture of E- and Z-isomers. The Product (II) was then characterized by ¹H- and ¹³C-NMR spectroscopy.

Both E- and Z-isomers can be synthesized through the above synthetic scheme. Modifications of the above procedure can be used to prepare additional compounds of the invention. For example, alternative methods for preparing substituted alkenes can be used to prepare variants of Intermediate (I).

### Example 2

### Inhibition of Jak2-V617F Dependent HEL Cell Growth

Methods: Jak2-V617F expressing Human Erythroleukemia (HEL) cells were plated in 96 well plates at ˜40,000 cells per well and incubated with either vehicle control (0.25% DMSO) or with 25 uM of Jak2 kinase inhibitors. Viable cell numbers were then determined at the times (0, 4, 24, 48 and 72 hours) via trypan blue exclusion and a hemocytometer. Aqueous solubility was determined using a MultiScreen® Solubility Filter Plate (Millipore) (see Quantitative method to determine drug aqueous solubility: optimization and correlation to standard methods; http://www.millipore.com/techpublications/tech1/an1730en00) and a Spectrophotometer.

The Results Growth inhibition and aqueous solubility for various Jak2 kinase inhibitors tested are summarized in Table 1 as follows:

TABLE 1

| | % Growth | Aqueous |
|---|---|---|
| Compound | Inhibition | Solubility (μM) |
| G6 | 100% | 455 |
| NB-1 | 98% | 533 |
| NB-2 | 53% | 816 |
| NB-3 | 101% | 460 |
| NB-4 | 101% | 478 |
| NB-5 | 101% | 509 |
| NB-6 | 19% | 538 |
| NB-7 | 99% | 495 |
| NB-8 | 100% | 272 |
| NB-9 | 101% | 384 |
| NB-10 | 100% | 474 |
| NB-12 | 100% | 484 |

Table 1 shows that various compounds had good cell inhibition similar to G6 (except that NB-2 and NB-6 had relatively moderate cell inhibition, ˜53% and ˜19% of G6, respectively). Table 1 also demonstrates that the aqueous solubility values for most of the compounds tested were between about 400 to 500 μM.

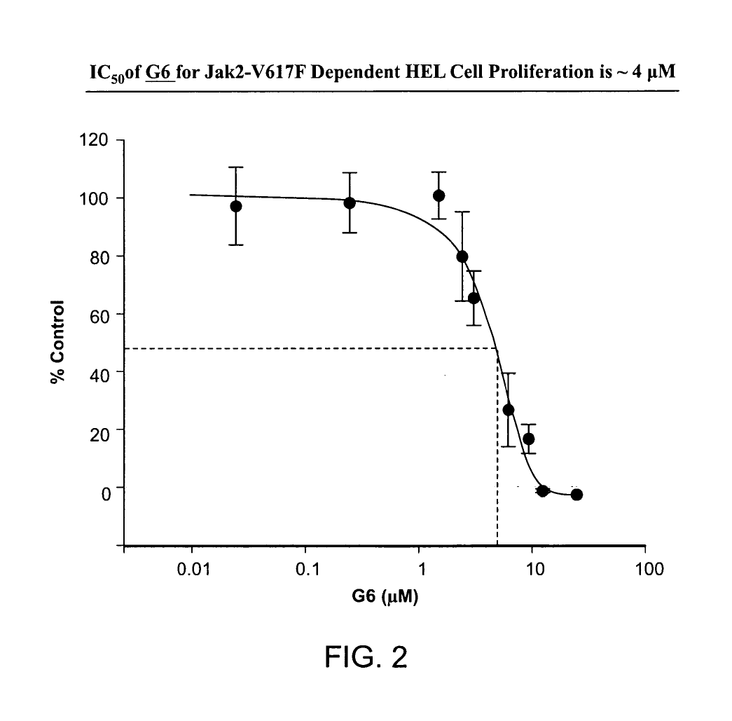

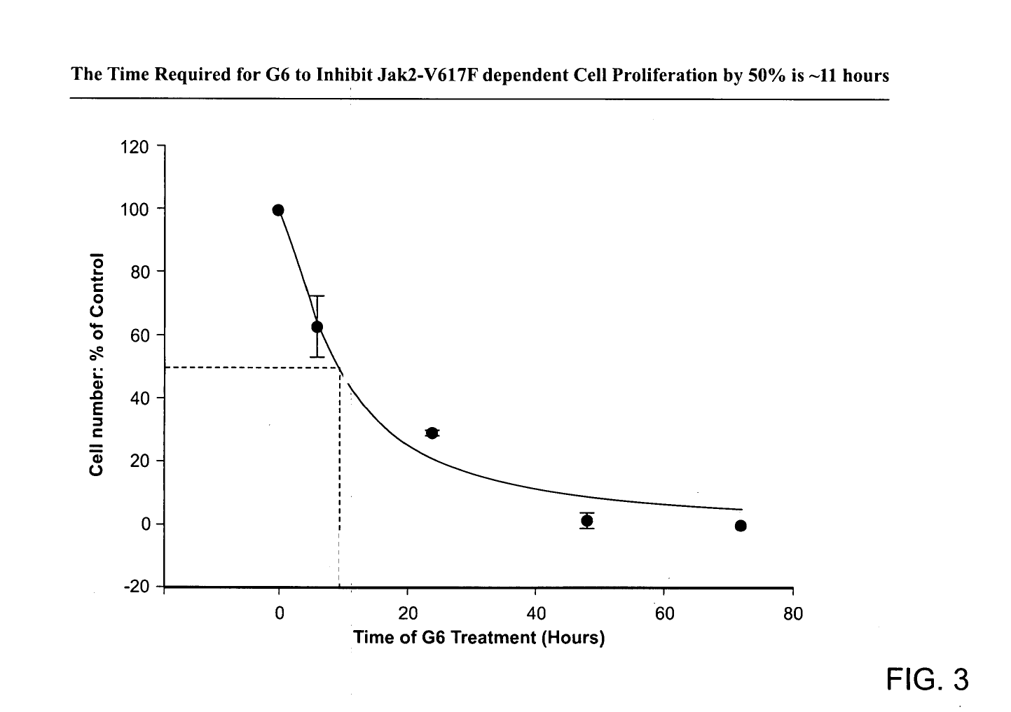

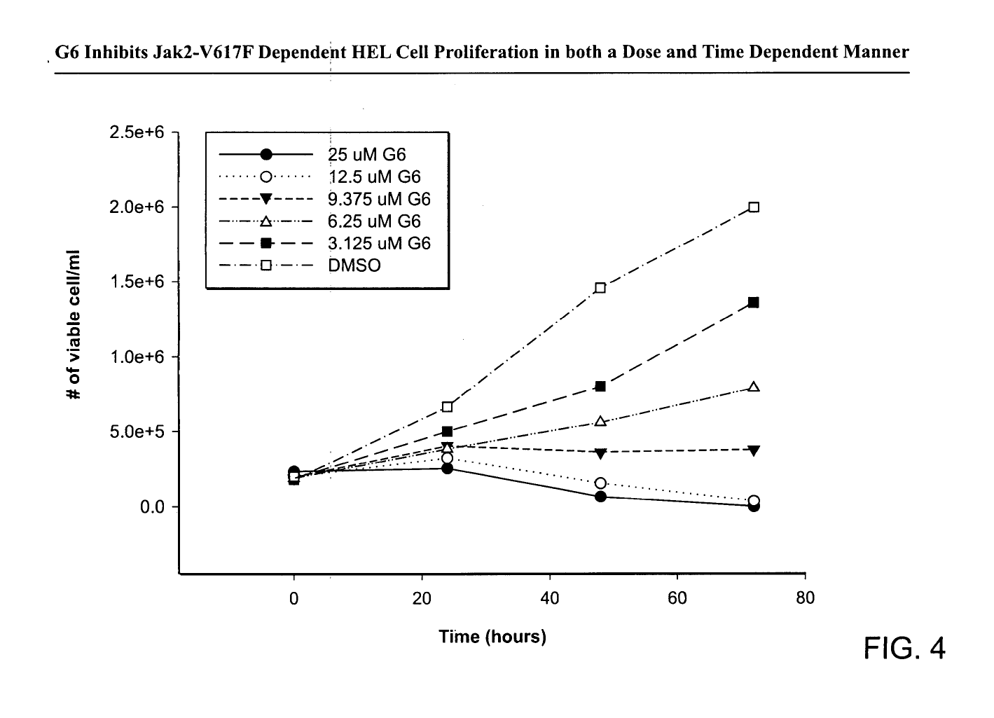

Further, test results on G6 are summarized in FIGS. 2, 3, and 4. FIG. 2 shows that IC₅₀ of G6 for inhibition on Jak2-V617F dependent HEL cells proliferation is about 4 μM. FIG. 3 demonstrates that the time required for G6 to inhibit Jak2-V617F dependent cell Proliferation by 50% is ˜11 hours. FIG. 4 shows that G6 Inhibits Jak2-V617F Dependent HEL Cell Proliferation in both a dose and time dependent manner.

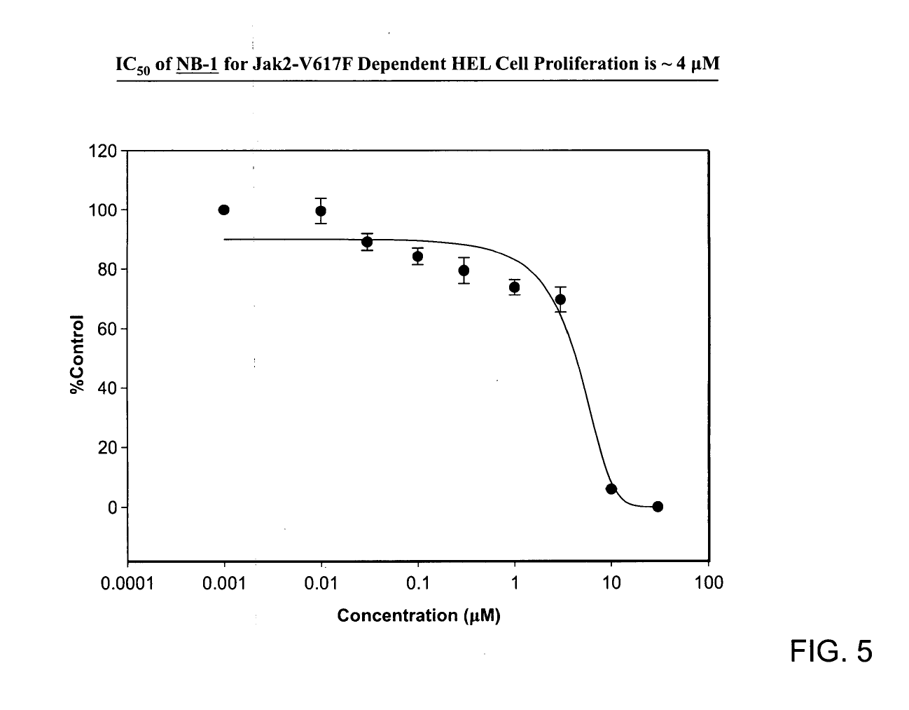

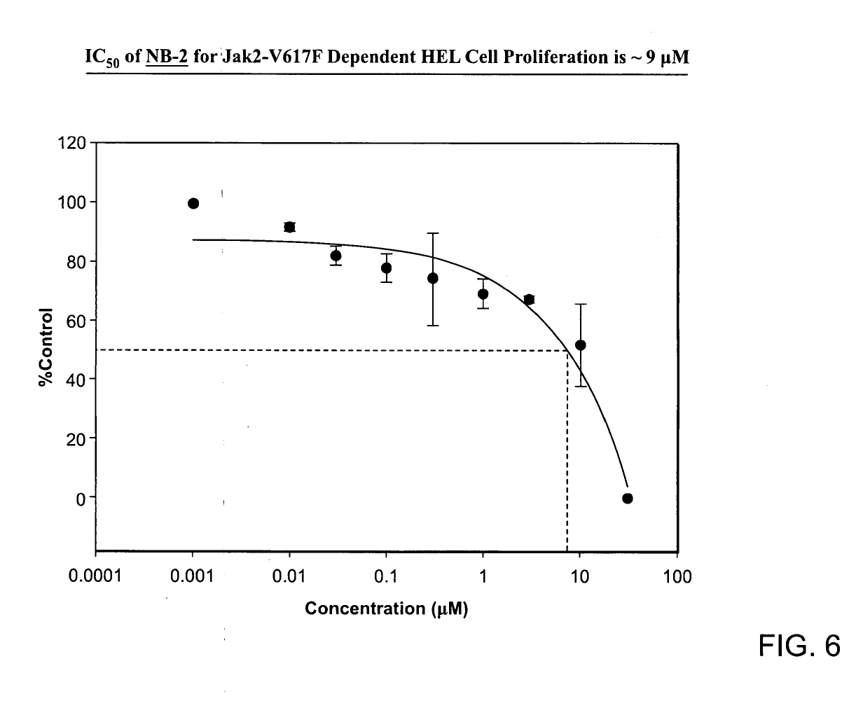

Test results on NB-1 and NB-2 are summarized in FIGS. 5 and 6 respectively. FIG. 5 shows that IC₅₀ of NB-1 for inhibition on Jak2-V617F dependent HEL cells proliferation is about 4 μM. FIG. 6 demonstrates that IC₅₀ of NB-2 for inhibition on Jak2-V617F dependent HEL cells proliferation is about 9 μM.

### Example 3

### Ex Vivo Study on Jak2 Kinase Inhibitors on Pathologic Cell Growth

Methods: Marrow derived mononuclear cells were obtained from a 60-year old female, who has been confirmed to have polycythemia vera and also identified as Jak2-V617F positive. Marrow derived mononuclear cells were washed three times in IMDM media and plated at 4×105 cells/mL in 1 mL methylcellulose media (0.9% methylcellulose, 30% heat inactivated FCS, 0.1 mM 2-mercaptoethanol, 0.9% BSA, 0.05% NaHCO₃, 2 mM/L glutamine, penicillin, streptomycin, 50 ng/mL SCF, and 20 ng/mL IL-3 (Stem Cell Technologies, Vancouver, BC). Vehicle control (DMSO at 0.25%) or inhibitors were added at the indicated concentrations (25 or 2.5 μM). EPO (1 U/mL) was also added as indicated. The cultures were then incubated at 37° C. and 5% CO₂ until assessment of colony formation at day 14. Results were expressed as the average number of colonies from duplicate cultures per 4×10⁵ cells.

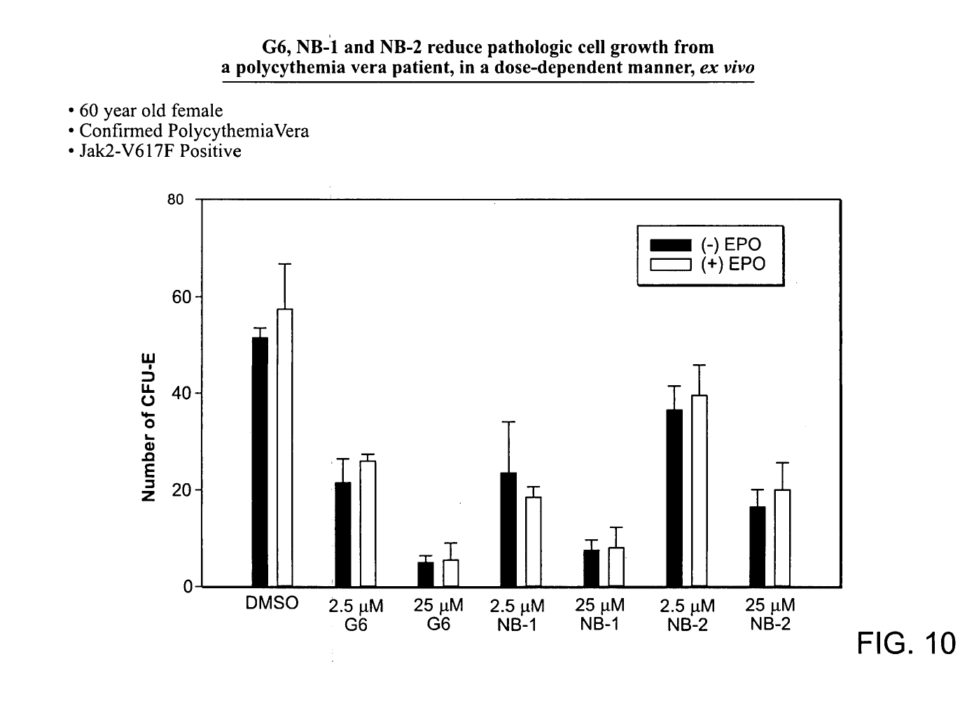

Results: Test results on G6, NB-1 and NB-2 are summarized in FIG. 10, which demonstrates that G6, NB-1 and NB-2 reduce pathologic cell growth.

### Example 4

### Assay to Demonstrate the Therapeutic Efficacy of an Inhibitor in Jak2-V61F-Induced Hematopoietic Disease in a NOD-SCID Mouse Model

Methods: In the experiment, NOD-SCID mice, N=36. were randomized into 6 groups (n=6). Baseline peripheral blood samples and weights were taken at Day 0. At day 7, 2×10⁶ HEL cells were injected into each mouse in Groups 2, 3, 4 and 5. At day 28, Group 1 mice were completely naïve; Group 2 mice were dosed with DMSO; Group 3 mice were dosed with 0.1 mg/kg/day G6; Group 4 mice were dosed with 1.0 mg/kg/day G6; Group 5 mice were dosed with 10 mg/kg/day G6; Group 6 mice were dosed with 10 mg/kg/day G6, until day 49. Then all the mice were euthanized.

In the experiment, the following measurable endpoints were assessed. For peripheral blood, total white blood cell (WBC) counts, the percentage of blast cells, the percentage of nucleated red blood cells (RBC), and Hematocrits were measured. For bone marrow, quantitative cellularity, and Myeloid cell to erythroid cell (M:E) ratio were assessed. Further, toxicity in spleen, brain, kidney, liver, and lung using histological analysis is assessed.

Peripheral blood samples were obtained via weekly submandibular bleeds using a 21 gauge needle. Blood samples were smeared onto glass slides, stained, and dried. Peripheral blast cells on each slide were then tabulated using a pathology light microscope. The average percent of peripheral blast cells for each treatment group was then graphed as a function of time.

At necropsy, femurs from each animal were formalin fixed, de-calcified and then parrafin embedded. Parrafin sections were then made, stained with hematoxylin and eosin and dried. The number of myeloid and erythroid cells were then tabulated by a veterinary pathologist who was blind to the treatment groups. The ratio of myeloid to erythroid cells was then graphed as a function of treatment group. Further, the average number of mature and immature erythroid cells was then graphed as a function of treatment group.

Additionally, at necropsy, animals were weighed one final time and spleens were then removed from each mouse. The wet weight of each spleen was recorded and the spleen weight to body weight ratio was graphed as a function of treatment group.

Results: In summary, injection of HEL cells into the tail vein of SCID-NOD mice resulted in marked Jak2 pathogenesis as evidenced by 1) increased blasts cells in the peripheral blood; 2) increased number of erythroid cells in the bone marrow (erythroleukemia); and 3) decreased myeloid to erythroid ratio.

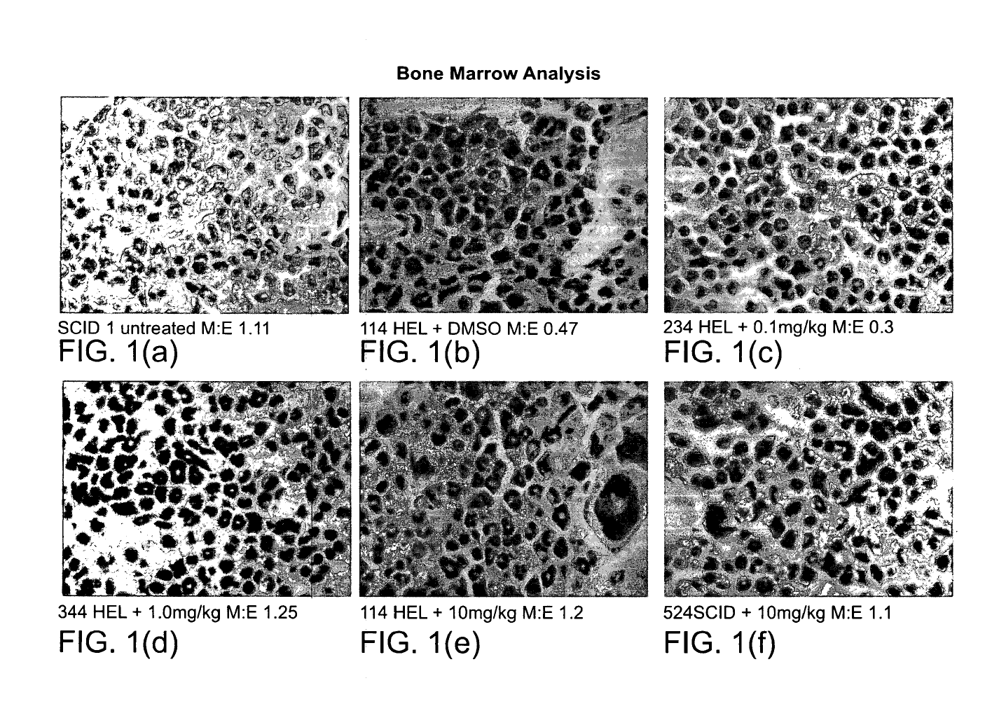

Test results on G6 are demonstrated in FIGS. 1, 11, 12, 13, and 14. FIG. 1 shows that a Bone Marrow Analysis: 1(a) depicts untreated SCID 1, wherein the ratio of Myeloid cells:Erythoid cells is 1.11; 1(b) depicts 114 HEL cell having been treated with DMSO, wherein the ratio of Myeloid cells:Erythoid cells is 0.47; 1(c) depicts 234A HEL cells having been treated with 0.1 mg/kg G6, wherein the ratio of Myeloid cells:Erythoid cells is 0.3; 1(d) depicts 344 HEL cells having been treated with 1.0 mg/kg G6, wherein the ratio of Myeloid cells:Erythoid cells is 1.25; 1(e) depicts 444 HEL cells having been treated with 10 mg/kg G6, wherein the ratio of Myeloid cells:Erythoid cells is 1.2; 1(f) depicts 524SCID having been treated with 10 mg/kg G6, wherein the ratio of Myeloid cells:Erythoid cells is 1.1.

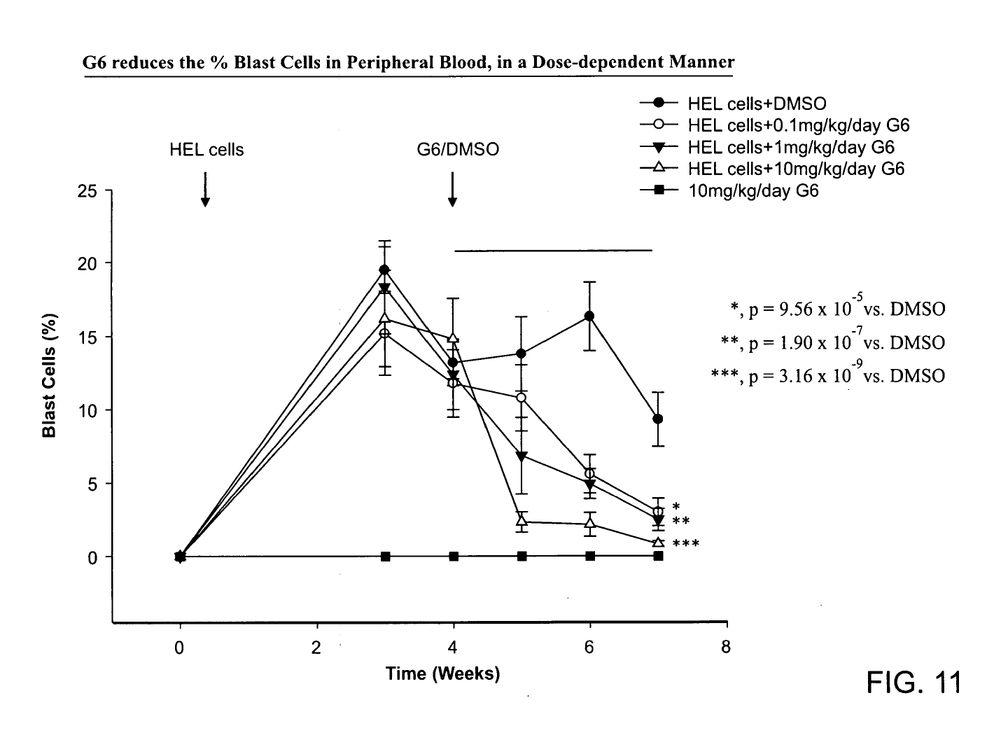

FIG. 11 shows that G6 reduces the percentage of blast cells in peripheral blood in a dose-dependent manner;

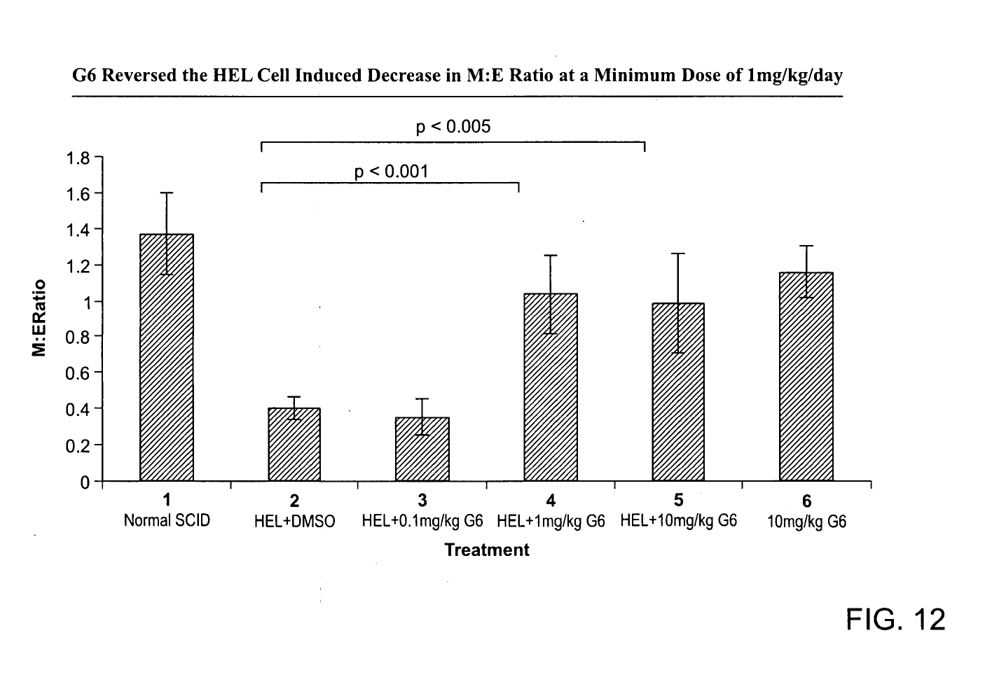

FIG. 12 shows that G6 reversed that HEL cell induced decrease in the ratio of Myeloid cells:Erythoid cells at a minimum dose of 1 mg/kg/day;

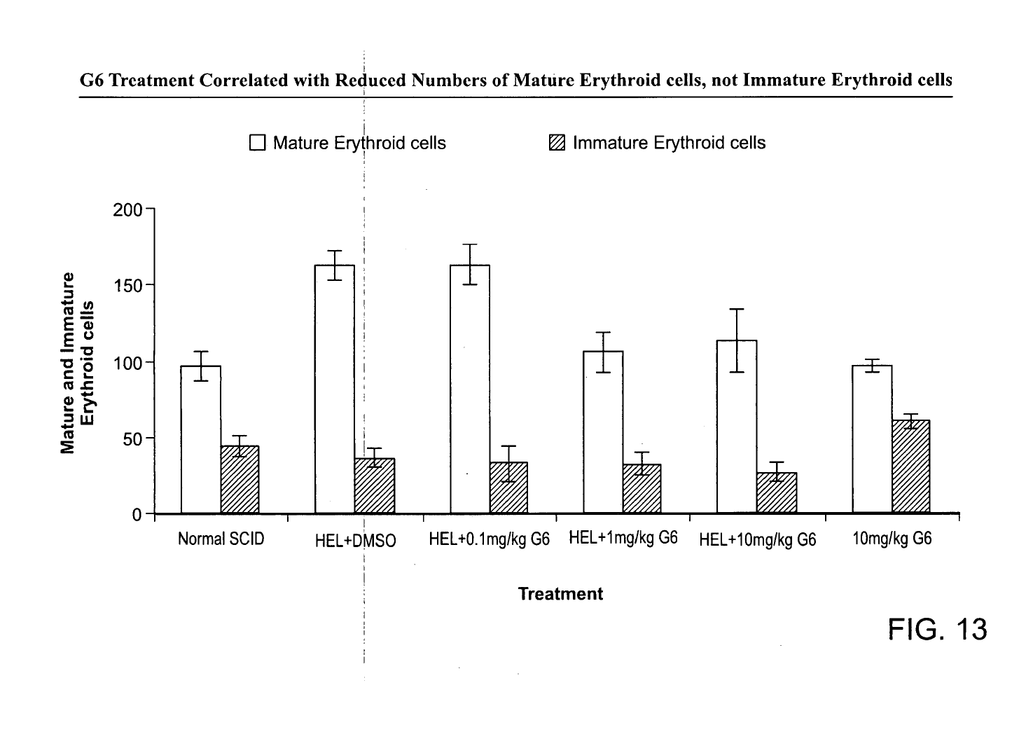

FIG. 13 demonstrates that G6 treatment correlates with reduced numbers of mature Erythroid cells, not immature Erythroid cells;

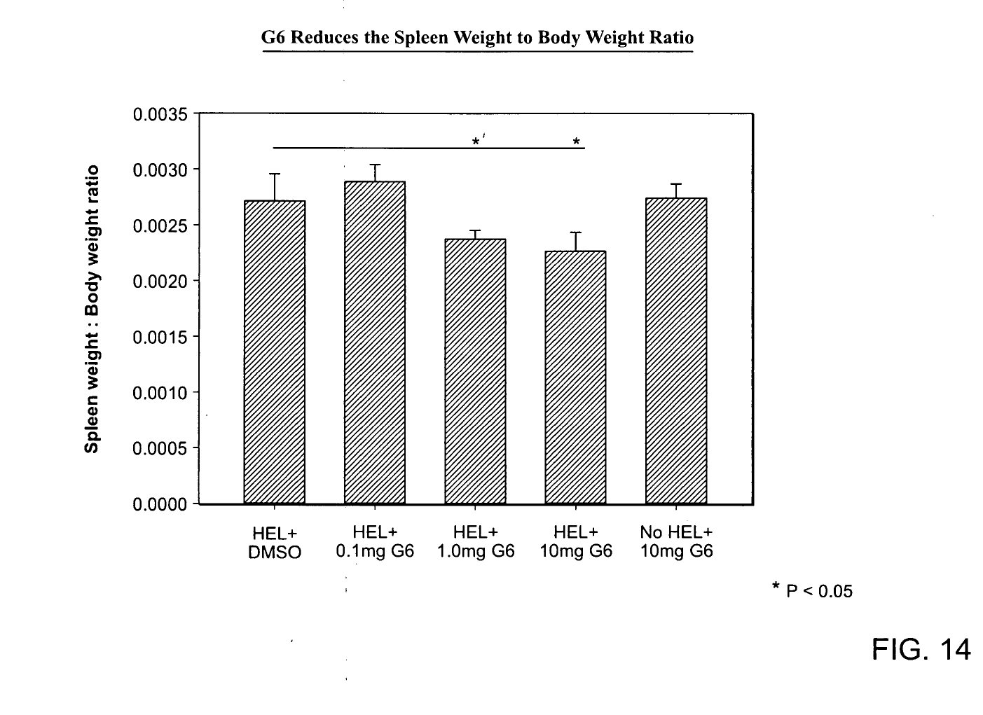

FIG. 14 shows that G6 reduces the spleen weight to body weight ratio.

Further, it was found that the 10 mg/kg/day dosage of G6 shows some degree of toxicity as indicated by bone marrow necrosis (2 of 6 mice) and splenic necrosis (3 of 6 mice); nevertheless, brain, kidney, liver, and lung of the animals were found to be histologically normal even in the mice at the 10 mg/kg/day dosage indicating that G6 is not globally toxic to tissues Peripheral blood counts of granulocytes, neutrophils, and eosinophils were all normal even in the mice at the 10 mg/kg/day dosage indicating that G6 is specific for erythroid progenitors.

Taken together, the results reported herein demonstrate that G6 treatment corrected Jak2-V617F mediated pathogenesis as evidenced by 1) decreased blasts cells in the peripheral blood; 2) decreased erythroid cells in the bone marrow; specifically, mature erythroid cells; 3) reversal of the pathological myeloid to erythroid ratio; and 4) reduced spleen weight to body weight ratio.

### Example 5

### c-Src Assay

Approximately 4 μL (12 units) of catalytically active recombinant p60c-src (Upstate Biotechnology) was incubated in 46 μL of in vitro kinase reaction buffer (50 mM HEPES, pH 7.6, 5 mM MnCl₂, 5 mM MgCl₂, 100 mM NaCl, 0.5 mM DTT), either in the presence of DMSO or 25 uM Z3. The reactions were incubated for 20 minutes at room temperature and then

terminated by addition of SDS-containing buffer. The samples were Western blotted with an anti-Src (pY418) polyclonal antibody (Biosource). The samples were subsequently immunoblotted with a cocktail of c-Src antibodies (Biosource, Upstate Biotechnology) at final dilutions of 1:1000 each to demonstrate equal c-Src protein among all samples.

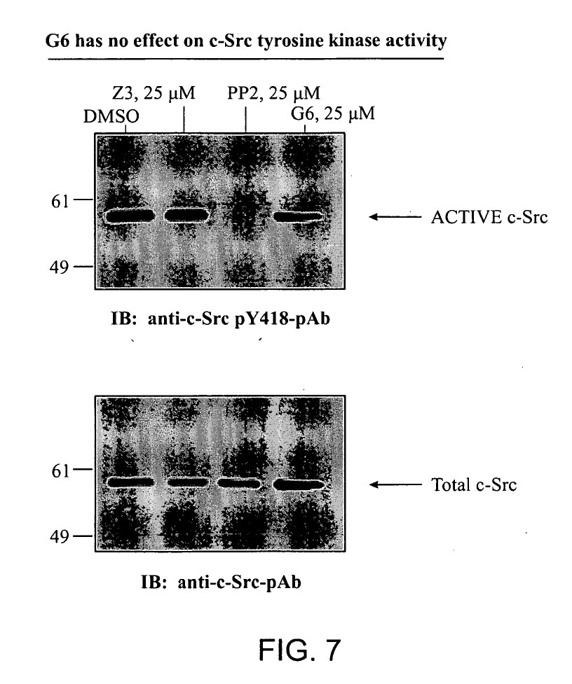

Results on G6 are summarized in FIG. 7, which shows that G6 has no effect on c-Src tyrosine kinase activity.

### Example 6

### Apoptosis Assay

Jak2-V617F expressing HEL cells were exposed to either vehicle control (DMSO), or 25 μM of an inhibitor for 48 hours. The percentage of apoptotic cells was then determined via Annexin V/Propidim Iodide FACS analysis.

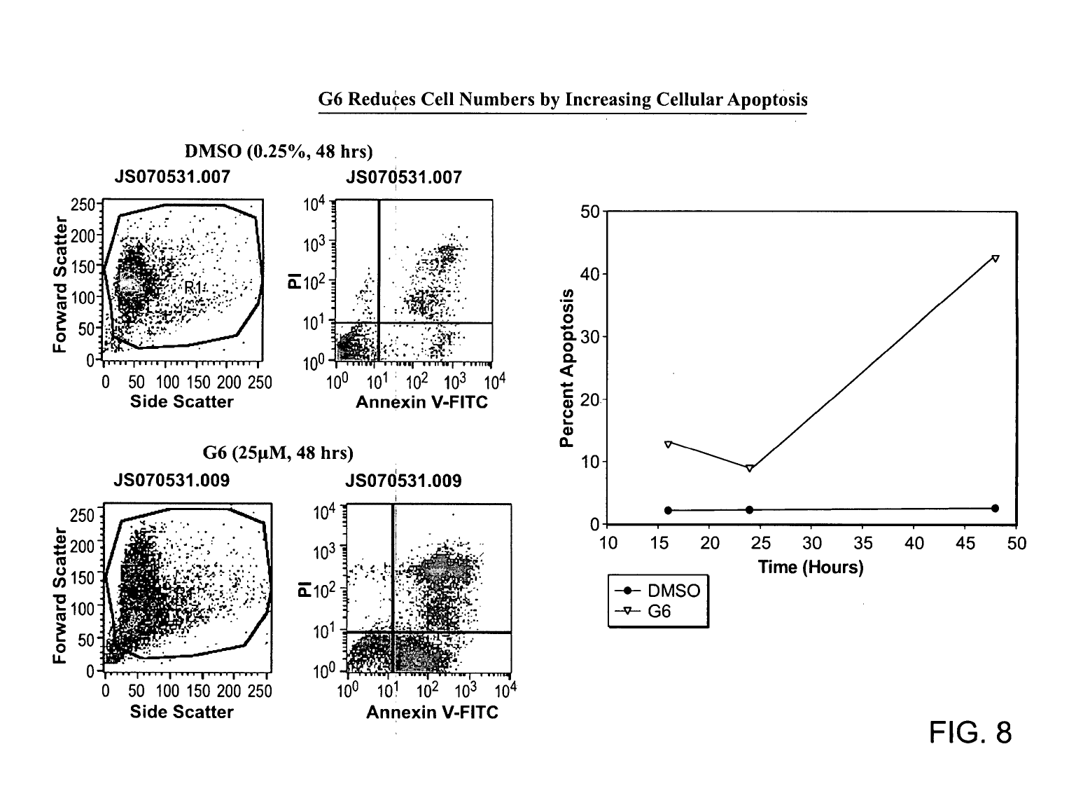

The test results on various compounds/conditions are summarized in Table 2. Further, test results on G6 is also demonstrated in FIG. 8.

TABLE 2

| Condition/ | % of Cells |
|---|---|
| compound | in Apoptosis |
| DMSO | 6% |
| G6 | 79% |
| NB-1 | 66% |
| NB-2 | 28% |

Table 2 also shows that NB-1 behaves similarly to G6 in suppressing HEL cell growth, while NB-2 is not as effective as G6. FIG. 8 demonstrates that G6 reduces cell numbers by increasing cellular apoptosis.

### Example 7

### Jak2-V617F Autophosphorylation Assay

BSC-40 cells were transfected in serum free media with 5.0 μg of a plasmid encoding the wild type murine Jak2 cDNA (pRC-CMV-Jak2-V617F) under the control of the bacteriophage T7 promoter, using Lipofectin and following the manufacturer's instructions (Invitrogen). Four hours later, the cells were infected with the recombinant vaccinia virus, vTF7-3, at a multiplicity of infection (MOI) of 1.0. One hour after that, the media containing Lipofectin/DNA/vTF7-3 was removed from the cells and replaced with fresh, serum-containing media. Inhibitor was added to the cells at this time at doses ranging from 10-100 μM. The cells were grown overnight at 37° C. to allow for high-level expression and subsequent tyrosine autophosphorylation of Jak2. Sixteen hour after the addition of the inhibitor, cells were washed with two volumes of ice-cold PBS containing 1 mM Na₃VO₄ and lysed in 0.8 ml ice-cold RIPA buffer containing protease inhibitors. The samples were sonicated and incubated on ice for 1 hour. Samples were spun at 16,000× g for 5 min at 4° C. and supernatants containing soluble protein lysates were normalized. Normalized lysates (approx. 400 ug/ml) were immunoprecipitated for 2-4 h at 4° C. with 2 μg of antibody and 20 ul of Protein A/G Plus agarose beads (Santa Cruz Biotechnology). After centrifugation, protein complexes were washed 3 times with wash buffer (25 mM Tris, pH 7.5, 150 mM NaCl, and 0.1% Triton X-100) and resuspended in SDS-containing sample buffer. Bound proteins were boiled, separated by SDS-PAGE, and transferred onto nitrocellulose membranes. The immunoprecipitating anti-Jak2-pAb (HR758) was from Santa Cruz Biotechnology. The immunoprecipitating anti-Tyr(P)-mAb (clone PY20) was from BD Transduction Laboratories. Phosphorylation levels were detected using enhanced chemiluminescence. Anti-Tyr(P) Western blotting was performed using a cocktail of antibodies consisting of clones 4G10 (Upstate Biotechnology), PY99 (Santa Cruz Biotecnology) and PY20 (BD Transduction Laboratories) at final dilutions of 1:1000 each. The anti-Jak2 antibody (758-776) was from Upstate Biotechnology (Millipore).

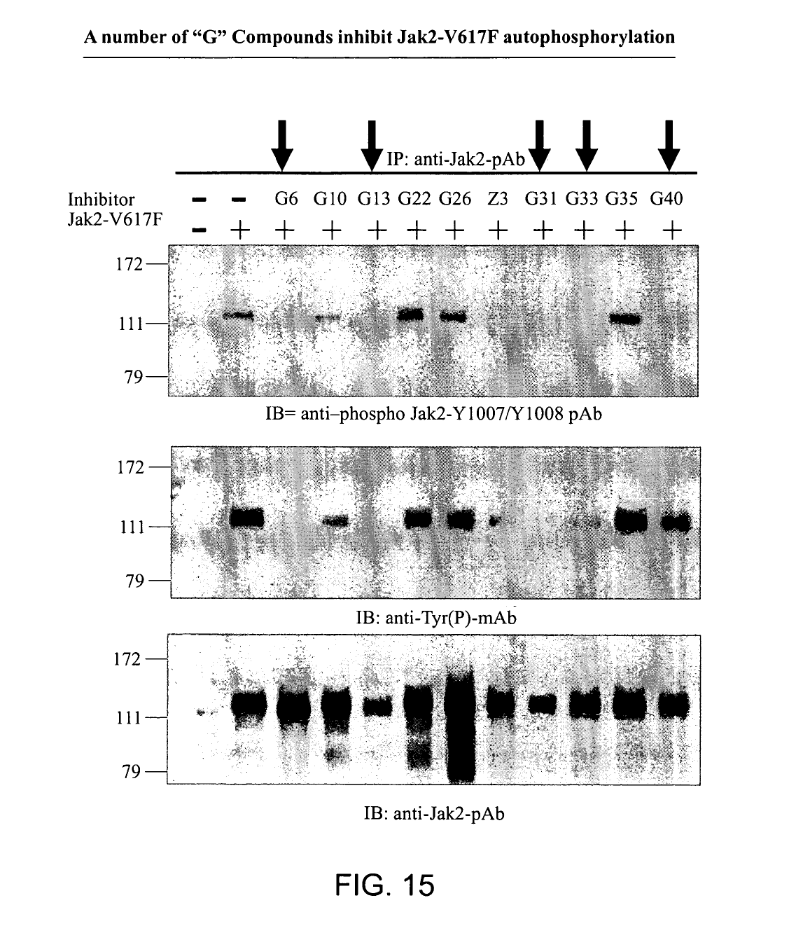

The test results on various compounds tested in Jak2-V617F autophorylation assay are summarized in FIG. 15.

### Example 8

### Ex Vivo Tests to Demonstrate that Jak2-Inhibitors Block Jak2-V617f Dependent Megakaryocyte Colony Formation

Marrow derived mononuclear cells were taken from a 61-year old male, who was confirmed Essential Thrombocythemia and being Jak2-V617F positive. The cells were washed three times in IMDM media and plated at 4×10⁵ cells/mL in 1 mL methylcellulose media (0.9% methylcellulose, 30% heat inactivated FCS, 0.1 mM 2-mercaptoethanol, 0.9% BSA, 0.05% NaHCO₃, 2 mM/L glutamine, penicillin, streptomycin, 50 ng/mL SCF, and 20 ng/mL IL-3 (Stem Cell Technologies, Vancouver, BC). Vehicle control (DMSO at 0.25%) or G6 (25 uM) was added as indicated. TPO (1 U/mL) was also added as indicated. The cultures were then incubated at 37° C. and 5% CO₂ until assessment of colony formation at day 14. Results were expressed as the average number of colonies from duplicate cultures per 4×10⁵ cells.

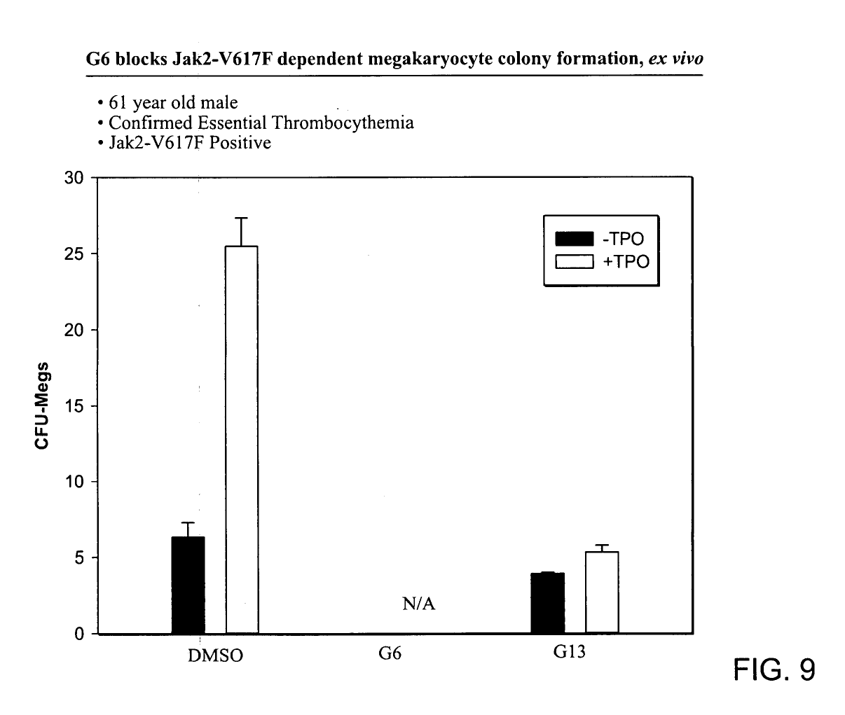

The test result on G6 is summarized in FIG. 9, which clearly demonstrates that G6 blocks Jak2-V617F dependent megakaryocyte colony formation.

### Example 9

Bone Marrow Immunohistochemistry: Immunochemistry was carried out on tissue fixed in 10% neutral-buffered formalin and paraffin-embedded. For detection of active STATS, mouse monoclonal anti-phospho-STAT5a/b (Y694/99; Advantex BioReagents LLP) was diluted 1:500 and incubated on sections overnight at 4° C. Detection of the antigen-antibody complexes was done by biotinylated secondary antibodies and streptavidin-peroxidase complex (DAKO). Hematoxylin was used for counterstaining. Antigen retrieval was done by heating (95° C., 20 min) with the BioGenex AR10 retrieval buffer. The staining intensity was quantified using the NIS-Element D software. Apoptotic cells were identified via the TUNEL (Terminal deoxynucleotidyl transferase-mediated dUTP-biotin nick end labeling) method, which specifically labels the 3′-hydroxyl termini of DNA strand breaks. For the TUNEL procedure, all reagents, including buffers, were part of the ApopTag Kit (Millipore). TUNEL positive cells appeared as highly stained, brown nuclei against the methyl green counterstain.

### Example 10

Phospho-STAT Analysis: Phospho- STAT1 [pY701], STAT3 [pY705], and STAT5a/b [pY694/699] ([pY694] for STAT5a and [pY699] for STAT5b) were measured using the STAT1, 3, 5a/b Phospho 3-Plex assay kit, a solid phase sandwich immunoassay, following the manufacturer's instructions (Invitrogen). The spectral properties of the 3 bead regions specific for each analyte were monitored with a Luminex® 100™ instrument.

The recitation of a listing of chemical groups in any definition of a variable herein includes definitions of that variable as any single group or combination of listed groups. The recitation of an element, an embodiment herein includes that element or embodiment as any single element or embodiment or in combination with any other element, embodiments or portions thereof.

The disclosures of each and every patent, patent application and publication cited herein are hereby incorporated herein by reference in their entirety.

Although the invention has been disclosed with reference to specific embodiments, it is apparent that other embodiments and variations of the invention may be devised by others skilled in the art without departing from the true spirit and scope of the invention. The claims are intended to be construed to include all such embodiments and equivalent variations.

We claim:

1. A compound of Formula (I): 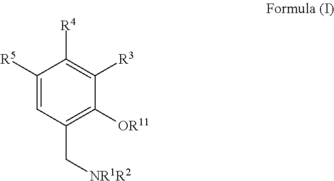
wherein
R¹ and R² are each independently H, —(C₁-C₄)alkyl, —(C₂-C₈)alkenyl, —(C₂-C₈)alkynyl, 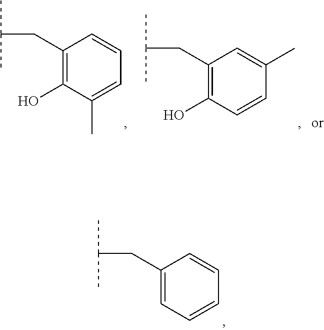
wherein —(C₁-C₄)alkyl can be further substituted with one or more hydroxy or halogen;
or
R¹ and R², together with the N-atom to which they are attached, to form a 5-membered or 6-membered heterocyclic ring, provided that when R¹ and R² together with the N-atom form a piperazine ring, the second nitrogen on the piperazine ring can be further substituted with —(C₁-C₄)alkyl, —(C₃-C₇)cycloalkyl, aryl or acyl, wherein —(C₁-C₄)alkyl, —(C₃-C₇)cycloalkyl, aryl or acyl can be substituted with one or more hydroxy, halogen or —(C₁-C₃)alkyl;
R³ is H, —(C₁-C₄)alkyl, —(C₃-C₇)cycloalkyl, aryl;
R⁴ is H or R⁷;
R⁵ is H, —(C₁-C₄)alkyl, —C(CH₃)₂—R⁶, or R⁷; provided that when R⁴ is H, R⁵ is R⁷ or —C(CH₃)₂—R⁶, and that when R⁵ is H or —(C₁-C₄)alkyl, R⁴ is R⁷, wherein R⁴ and R⁵ cannot be both R⁷ at the same time;
R⁶ is H, —(C₁-C₄)alkyl, phenyl, or 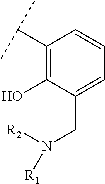
wherein R¹ and R² are as defined above;
R⁷ is 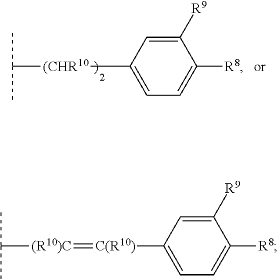
wherein R⁸ and R⁹ are each independently H, —OH, —O—(C₁-C₄)alkyl, —CH₂—NR¹R², wherein R¹ and R² are as defined above;
R¹⁰ for each occurrence independently is hydrogen, or —(C₁-C₃)alkyl;
R¹¹ is H, acyl, tosyl, —(C₁-C₄)alkyl, or aryl;
or a pharmaceutically acceptable salt, ester, hydrate or solvate thereof;
provided that the compound is not:
I. 4,4′-(Hex-3-ene-3,4-diyl)bis(2-((diethylamino)methyl)phenol); or
II. 4,4′-(Ethene-1,2-diyl)bis(2-((diethylamino)methyl)phenol); or
III. 5,5′-(Hex-3-ene-3,4-diyl)bis(2-((dimethylamino)methyl)phenol); or
IV. 4,4′-(Hex-3-ene-3,4-diyl)bis(2-((dimethylamino)methyl)phenol); or
V. 4,4′-(Ethene-1,2-diyl)bis(2-((dimethylamino)methyl)phenol); or
VI. 4,4′-(Hex-3-ene-3,4-diyl)bis(2-((diethylamino)methyl)phenol); or
VII. 4,4′-(Ethene-1,2-diyl)bis(2-((diethylamino)methyl)phenol); or
VIII. 4,4′-(Ethene-1,2-diyl)bis(2-(pyrrolidin-1-ylmethyl)phenol).

2. The compound of claim 1, wherein R¹⁰ is H, methyl or ethyl, and R¹¹ is H.

3. The compound of claim 2, wherein R³ is H, and R⁷ is

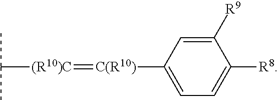

4. The compound of claim 3, wherein R⁴ is R⁷ and R⁵ is H.

5. The compound of claim 4, wherein R⁸ is —CH₂—NR¹R² and R⁹ is hydroxyl.

6. The compound of claim 5, wherein R¹⁰ for each occurrence independently is hydrogen or methyl.

7. The compound of claim 6, wherein R¹ and R² for each occurrence independently are —(C₁-C₄)alkyl; or R¹ and R² together with the N-atom to which they are attached form a piperidinyl, pyrrolidinyl or imidazolyl ring, provided that R¹⁰ is the same for each occurrence.

8. The compound of claim 5, wherein R¹⁰ is ethyl, R¹ and R² are ethyl or isopropyl; or R¹ and R² together with the N-atom to which they are attached form a pyrrolidinyl or imidazolyl ring.

9. The compound of claim 3, wherein R⁴ is H, and R⁵ is R⁷.

10. The compound of claim 9, wherein R⁸ is hydroxyl and R⁹ is —CH₂—NR¹R².

11. (canceled)

12. (canceled)

13. The compound of claim 10, wherein R¹⁰ is H or ethyl, R¹ and R² are propyl or isopropyl, provided that R¹⁰ is the same for each occurrence.

14. The compound of claim 10, wherein R¹⁰ is ethyl, R¹ and R² together with the N-atom to which they are attached form a piperidinyl, pyrrolidinyl or imidazolyl ring.

15. (canceled)

16. The compound of claim 1, wherein the compound is selected from the group of
a) (Z) and (E)-4,4′-(but-2-ene-2,3-diyl)bis(2-((diethylamino)methyl)phenol);
b) (Z) and (E)-5,5′-(but-2-ene-2,3-diyl)bis(2-((diethylamino)methyl)phenol);
c) (Z) and (E)-5,5′-(but-2-ene-2,3-diyl)bis(2-((dimethylamino)methyl)phenol);
d) (Z) and (E)-5,5′-(ethene-1,2-diyl)bis(2-((diethylamino)methyl)phenol);
e) (Z) and (E)-5,5′-(ethene-1,2-diyl)bis(2-((dimethylamino)methyl)phenol);
f) (Z) and (E)-5,5′-(ethene-1,2-diyl)bis(2-(piperidin-1-ylmethyl)phenol);
g) (Z) and (E)-5,5′-(but-2-ene-2,3-diyl)bis(2-((diethylamino)methyl)phenol).2HCl;
h) (Z) and (E)-5,5′-(but-2-ene-2,3-diyl)bis(2-((dimethylamino)methyl)phenol).2HCl;
i) (Z) and (E)-5,5′-(ethene-1,2-diyl)bis(2-((diethylamino)methyl)phenol).2HCl;
j) (Z) and (E)-5,5′-(ethene-1,2-diyl)bis(2-((dimethylamino)methyl)phenol).2HCl;
k) (Z) and (E)-5,5′-(ethene-1,2-diyl)bis(2-(piperidin-1-ylmethyl)phenol).2HCl;
l) (Z) and (E)-5,5′-(but-2-ene-2,3-diyl)bis(2-((dimethylamino)methyl)phenol);
m) (Z) and (E)-5,5′-(ethene-1,2-diyl)bis(2-((dimethylamino)methyl)phenol);
n) (Z) and (E)-4,4′-(but-2-ene-2,3-diyl)bis(2-((dimethylamino)methyl)phenol);
o) (Z) and (E)-5,5′-(but-2-ene-2,3-diyl)bis(2-((diethylamino)methyl)phenol);
p) (Z) and (E)-5,5′-(hex-3-ene-3,4-diyl)bis(2-((diethylamino)methyl)phenol);
q) (Z) and (E)-5,5′-(ethene-1,2-diyl)bis(2-((diethylamino)methyl)phenol);
r) (Z) and (E)-4,4′-(but-2-ene-2,3-diyl)bis(2-((diethylamino)methyl)phenol);
s) (Z) and (E)-5,51-(But-2-ene-2,3-diyl)bis(2-(pyrrolidin-1-ylmethyl)phenol);
t) (Z) and (E)-5,51-(Hex-3-ene-3,4-diyl)bis(2-(pyrrolidin-1-ylmethyl)phenol);
u) (Z) and (E)-5,5′-(Ethene-1,2-diyl)bis(2-(pyrrolidin-1-ylmethyl)phenol);
v) (Z) and (E)-4,4′-(But-2-ene-2,3-diyl)bis(2-(pyrrolidin-1-ylmethyl)phenol);
w) (Z) and (E)-4,4′-(Hex-3-ene-3,4-diyl)bis(2-(pyrrolidin-1-ylmethyl)phenol);
x) (Z) and (E)-5,5′-(But-2-ene-2,3-diyl)bis(2-((diisopropylamino)methyl)phenol);
y) (Z) and (E)-5,51-(Hex-3-ene-3,4-diyl)bis(2-((diisopropylamino)methyl)phenol);
z) (Z) and (E)-5,5′-(Ethene-1,2-diyl)bis(2-((diisopropylamino)methyl)phenol);
aa) (Z) and (E)-4,4′-(But-2-ene-2,3-diyl)bis(2-((diisopropylamino)methyl)phenol);
bb) (Z) and (E)-4,4′-(But-2-ene-2,3-diyl)bis(2-((diisopropylamino)methyl)phenol);
cc) (Z) and (E)-4,4′-(Hex-3-ene-3,4-diyl)bis(2-((diisopropylamino)methyl)phenol);
dd) (Z) and (E)-4,4′-(Ethene-1,2-diyl)bis(2-((diisopropylamino)methyl)phenol);
ee) (Z) and (E)-5,5′-(But-2-ene-2,3-diyl)bis(2-((1H-imidazol-1-yl)methyl)phenol);
ff) (Z) and (E)-5,5′-(Hex-3-ene-3,4-diyl)bis(2-((1H-imidazol-1-yl)methyl)phenol);
gg) (Z) and (E)-5,5′-(Ethene-1,2-diyl)bis(2-((1H-imidazol-1-yl)methyl)phenol);
hh) (Z) and (E)-4,4′-(But-2-ene-2,3-diyl)bis(2-((1H-imidazol-1-yl)methyl)phenol);
ii) 4,4′-(Hex-3-ene-3,4-diyl)bis(2-((1H-imidazol-1-yl)methyl)phenol); and
jj) (Z) and (E)-4,4′-(Ethene-1,2-diyl)bis(2-((1H-imidazol-1-yl)methyl)phenol);
or a pharmaceutically acceptable salt, ester, hydrate or solvate thereof.

17. The compound of claim 1, wherein the compound is selected from the group consisting of
1) (Z) and (E)-4,4′-(Ethene-1,2-diyl)bis(2-((diethylamino)methyl)phenol);
2) (Z) and (E)-4,4′-(But-2-ene-2,3-diyl)bis(2-((diethylamino)methyl)phenol);
3) (Z) and (E)-5,5′-(But-2-ene-2,3-diyl)bis(2-((diethylamino)methyl)phenol);
4) (Z) and (E)-5,5′-(But-2-ene-2,3-diyl)bis(2-((dimethylamino)methyl)phenol);
5) (Z) and (E)-5,5′-(Ethene-1,2-diyl)bis(2-((diethylamino)methyl)phenol);
6) (Z) and (E)-5,5′-(Ethene-1,2-diyl)bis(2-((dimethylamino)methyl)phenol);
7) (Z) and (E)-5,5′-(Ethene-1,2-diyl)bis(2-(piperidin-1-ylmethyl)phenol);
8) (Z) and (E)-5,5′-(But-2-ene-2,3-diyl)bis(2-((diethylamino)methyl)phenol).2HCl;
9) (Z) and (E)-5,5′-(But-2-ene-2,3-diyl)bis(2-((dimethylamino)methyl)phenol).2HCl;
10) (Z) and (E)-5,5′-(Ethene-1,2-diyl)bis(2-((diethylamino)methyl)phenol).2HCl;
11) (Z) and (E)-5,5′-(Ethene-1,2-diyl)bis(2-((dimethylamino)methyl)phenol).2HCl;
12) 5,5′-(Ethene-1,2-diyl)bis(2-(piperidin-1-ylmethyl)phenol);
13) (Z) and (E)-4,4′-(But-2-ene-2,3-diyl)bis(2-((dimethylamino)methyl)phenol);
14) (Z) and (E)-5,5′-(Ethene-1,2-diyl)bis(2-(pyrrolidin-1-ylmethyl)phenol);
15) (Z) and (E)-4,4′-(Ethene-1,2-diyl)bis(2-((dimethylamino)methyl)phenol);
16) (Z) and (E)-5,5-(But-2-ene-2,3-diyl)bis(2-(piperidin-1-ylmethyl)phenol);
17) (Z) and (E)-5,5′-(but-2-ene-2,3-diyl)bis(2-(pyrrolidin-1-ylmethyl)phenol);
18) (Z) and (E)-5,5′-(Ethene-1,2-diyl)bis(2-(morpholinomethyl)phenol);
19) (Z) and (E)-5,5′-(But-2-ene-2,3-diyl)bis(2-(morpholinomethyl)phenol);
20) (Z) and (E)-4,4′-(Ethene-1,2-diyl)bis(2-(piperidin-1-ylmethyl)phenol);
21) (Z) and (E)-4,4′-(Ethene-1,2-diyl)bis(2-(pyrrolidin-1-ylmethyl)phenol);
22) (Z) and (E)-4,4′-(Ethene-1,2-diyl)bis(2-(morpholinomethyl)phenol);
23) (Z) and (E)-4,4′-(Hex-3-ene-3,4-diyl)bis(2-((dimethylamino)methyl)phenol);
24) (Z) and (E)-4,4′-(Hex-3-ene-3,4-diyl)bis(2-(pyrrolidin-1-ylmethyl)phenol);
25) (Z) and (E)-4,4′-(Hex-3-ene-3,4-diyl)bis(2-(piperidin-1-ylmethyl)phenol);
26) (Z) and (E)-4,4′-(Hex-3-ene-3,4-diyl)bis(2-(morpholinomethyl)phenol);
27) (Z) and (E)-4,4′-(But-2-ene-2,3-diyl)bis(2-(piperidin-1-ylmethyl)phenol);
28) (Z) and (E)-4,4′-(But-2-ene-2,3-diyl)bis(2-(pyrrolidin-1-ylmethyl)phenol);
29) (Z) and (E)-2-((Diethylamino)methyl)-4-(4-(4-hydroxyphenyl)hex-3-en-3-yl)phenol;
30) (Z) and (E)-4,4′-(Hex-3-ene-3,4-diyl)diphenol;
31) (Z) and (E)-4,4′-(But-2-ene-2,3-diyl)diphenol;
32) (Z) and (E)-3,3′-(Ethene-1,2-diyl)diphenol;
33) (Z) and (E)-3,3′-(But-2-ene-2,3-diyl)diphenol; and
34) (Z) and (E)-4,4′-(Ethene-1,2-diyl)diphenol;
or a pharmaceutically acceptable salt, ester, hydrate or solvate thereof.

18. A method of treating or preventing a Jak-2 mediated disease or disorder in a subject, the method comprising administering to the subject an effective amount of a compound of claim 1.

19. (canceled)

20. (canceled)

21. The method of claim 18, wherein the Jak2-mediated disease or disorder is polycythemia vera, essential thrombocythemia, or angiogenic myeloid metaplasia.

22. (canceled)

23. (canceled)

24. (canceled)

25. (canceled)

26. The method of claim 18, wherein the compound of claim 1 is selected from the group of 4,4′-(Hex-3-ene-3,4-diyl)bis(2-((diethylamino)methyl)phenol); 4,4′-(Ethene-1,2-diyl)bis(2-((diethylamino)methyl)phenol); 4,4′-(but-2-ene-2,3-diyl)bis(2-((diethylamino)methyl)phenol); 5,5′-(but-2-ene-2,3-diyl)bis(2-((diethylamino)methyl)phenol); 5,5′-(but-2-ene-2,3-diyl)bis(2-((dimethylamino)methyl)phenol); 5,5′-(ethene-1,2-diyl)bis(2-((diethylamino)methyl)phenol); 5,5′-(ethene-1,2-diyl)bis(2-((dimethylamino)methyl)phenol); 5,5′-(ethene-1,2-diyl)bis(2-(piperidin-1-ylmethyl)phenol); 5,5′-(but-2-ene-2,3-diyl)bis(2-((diethylamino)methyl)phenol).2HCl; 5,5′-(but-2-ene-2,3-diyl)bis(2-((dimethylamino)methyl)phenol).2HCl; 5,5′-(ethene-1,2-diyl)bis(2-((diethylamino)methyl)phenol).2HCl; 5,5′-(ethene-1,2-diyl)bis(2-((dimethylamino)methyl)phenol).2HCl; 5,5′-(ethene-1,2-diyl)bis(2-(piperidin-1-ylmethyl)phenol).2HCl; 5,5′-(but-2-ene-2,3-diyl)bis(2-((dimethylamino)methyl)phenol); 5,5′-(ethene-1,2-diyl)bis(2-((dimethylamino)methyl)phenol); 4,4′-(but-2-ene-2,3-diyl)bis(2-((dimethylamino)methyl)phenol); 5,5′-(but-2-ene-2,3-diyl)bis(2-((diethylamino)methyl)phenol); 5,5′-(hex-3-ene-3,4-diyl)bis(2-((diethylamino)methyl)phenol); 5,5′-(ethene-1,2-diyl)bis(2-((diethylamino)methyl)phenol); 4,4′-(but-2-ene-2,3-diyl)bis(2-((diethylamino)methyl)phenol); 5,5′-(But-2-ene-2,3-diyl)bis(2-(pyrrolidin-1-ylmethyl)phenol); 5,5′-(Hex-3-ene-3,4-diyl)bis(2-(pyrrolidin-1-ylmethyl)phenol); 5,5′-(Ethene-1,2-diyl)bis(2-(pyrrolidin-1-ylmethyl)phenol); 4,4′-(But-2-ene-2,3-diyl)bis(2-(pyrrolidin-1-ylmethyl)phenol); 4,4′-(Hex-3-ene-3,4-diyl)bis(2-(pyrrolidin-1-ylmethyl)phenol); 5,5′-(But-2-ene-2,3-diyl)bis(2-((diisopropylamino)methyl)phenol); 5,5′-(Hex-3-ene-3,4-diyl)bis(2-((diisopropylamino)methyl)phenol); 5,5′-(Ethene-1,2-diyl)bis(2-((diisopropylamino)methyl)phenol); 4,4′-(But-2-ene-2,3-diyl)bis(2-((diisopropylamino)methyl)phenol); 4,4′-(But-2-ene-2,3-diyl)bis(2-((diisopropylamino)methyl)phenol); 4,4′-(Hex-3-ene-3,4-diyl)bis(2-((diisopropylamino)methyl)phenol); 4,4′-(Ethene-1,2-diyl)bis(2-((diisopropylamino)methyl)phenol); 5,5′-(But-2-ene-2,3-diyl)bis(2-((1H-imidazol-1-yl)methyl)phenol); 5,5′-(Hex-3-ene-3,4-diyl)bis(2-((1H-imidazol-1-yl)methyl)phenol); 5,5′-(Ethene-1,2-diyl)bis(2-((1H-imidazol-1-yl)methyl)phenol); 4,4′-(But-2-ene-2,3-diyl)bis(2-((1H-imidazol-1-yl)methyl)phenol); 4,4′-(Hex-3-ene-3,4-diyl)bis(2-((1H-imidazol-1-yl)methyl)phenol); 4,4′-(Ethene-1,2-diyl)bis(2-((1H-imidazol-1-yl)methyl)phenol); 5,5′-(Hex-3-ene-3,4-diyl)bis(2-((dimethylamino)methyl)phenol); 4,4′-(Hex-3-ene-3,4-diyl)bis(2-((dimethylamino)methyl)phenol); 4,4′-(Ethene-1,2-diyl)bis(2-((dimethylamino)methyl)phenol); 4,4′-(Hex-3-ene-3,4-diyl)bis(2-((diethylamino)methyl)phenol); 4,4′-(Ethene-1,2-diyl)bis(2-((diethylamino)methyl)phenol); and 4,4′-(Ethene-1,2-diyl)bis(2-(pyrrolidin-1-ylmethyl)phenol); or a pharmaceutically acceptable salt, ester, hydrate or solvate thereof.

27. (canceled)

28. (canceled)

29. (canceled)

30. A method of treating cancer in a subject, the method comprising administering to the subject an effective amount of a compound of claim 1.

31. (canceled)

32. (canceled)

33. (canceled)

34. (canceled)

35. A method of treating a hematological disease or disorder in a subject, the method comprising administering to the subject an effective amount of a compound of claim 1.

36. A pharmaceutical composition comprising a compound of claim 1, together with a pharmaceutically acceptable carrier.

37. (canceled)

38. (canceled)

39. A compound of Formula (III): 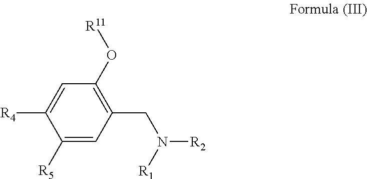
wherein
R¹ and R² are each independently H, —(C₁-C₄)alkyl, —(C₂-C₈)alkenyl, —(C₂-C₈)alkynyl, 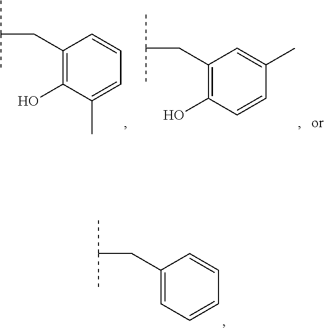
wherein —(C₁-C₄)alkyl can be further substituted with one or more hydroxy or halogen;
or
R¹ and R², together with the N-atom to which they are attached, to form a 5-membered or 6-membered heterocyclic ring, provided that when R¹ and R² together with the N-atom form a piperazine ring, the second nitrogen on the piperazine ring can be further substituted with —(C₁-C₄)alkyl, —(C₃-C₇)cycloalkyl, aryl or acyl, wherein —(C₁-C₄)alkyl, —(C₃-C₇)cycloalkyl, aryl or acyl can be substituted with one or more hydroxy, halogen or —(C₁-C₃)alkyl;
R¹¹ is H, acyl, tosyl, —(C₁-C₄)alkyl, or aryl;
R⁴ and R⁵ are H or R¹², provided that one of R⁴ and R⁵ is H, and the other is R¹²;
R¹² is 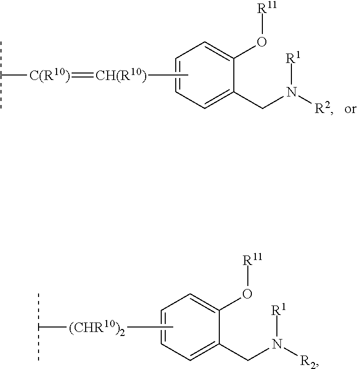
wherein the aryl group to which both R⁴ and R⁵ are attached is meta or para to the —OR¹¹ in the aromatic ring of R¹²;
R¹⁰ is hydrogen, or —(C₁-C₃)alkyl;
or a pharmaceutically acceptable salt, ester, hydrate or solvate thereof;
provided that the compound is not:
i. 4,4′-(Hex-3-ene-3,4-diyl)bis(2-((diethylamino)methyl)phenol); or
ii. 4,4′-(Ethene-1,2-diyl)bis(2-((diethylamino)methyl)phenol); or
iii. 5,5′-(Hex-3-ene-3,4-diyl)bis(2-((dimethylamino)methyl)phenol); or
iv. 4,4′-(Hex-3-ene-3,4-diyl)bis(2-((dimethylamino)methyl)phenol); or
v. 4,4′-(Ethene-1,2-diyl)bis(2-((dimethylamino)methyl)phenol); or
vi. 4,4′-(Hex-3-ene-3,4-diyl)bis(2-((diethylamino)methyl)phenol); or
vii. 4,4′-(Ethene-1,2-diyl)bis(2-((diethylamino)methyl)phenol); or
viii. 4,4′-(Ethene-1,2-diyl)bis(2-(pyrrolidin-1-ylmethyl)phenol).

40. A compound of claim 1 selected from the group consisting of
1) (Z) and (E)-4,4′-(Ethene-1,2-diyl)bis(2-((diethylamino)methyl)phenol);
2) (Z) and (E)-4,4′-(But-2-ene-2,3-diyl)bis(2-((diethylamino)methyl)phenol);
3) (Z) and (E)-5,5′-(But-2-ene-2,3-diyl)bis(2-((diethylamino)methyl)phenol);
4) (Z) and (E)-5,5′-(But-2-ene-2,3-diyl)bis(2-((dimethylamino)methyl)phenol);
5) (Z) and (E)-5,5′-(Ethene-1,2-diyl)bis(2-((diethylamino)methyl)phenol);
6) (Z) and (E)-5,5′-(Ethene-1,2-diyl)bis(2-((dimethylamino)methyl)phenol);
7) (Z) and (E)-5,5′-(Ethene-1,2-diyl)bis(2-(piperidin-1-ylmethyl)phenol);
8) (Z) and (E)-5,5′-(But-2-ene-2,3-diyl)bis(2-((diethylamino)methyl)phenol).2HCl;
9) (Z) and (E)-5,5′-(But-2-ene-2,3-diyl)bis(2-((dimethylamino)methyl)phenol).2HCl;
10) (Z) and (E)-5,5′-(Ethene-1,2-diyl)bis(2-((diethylamino)methyl)phenol).2HCl;
11) (Z) and (E)-5,5′-(Ethene-1,2-diyl)bis(2-((dimethylamino)methyl)phenol).2HCl; and
12) 5,5′-(Ethene-1,2-diyl)bis(2-(piperidin-1-ylmethyl)phenol);
or a pharmaceutically acceptable salt, ester, hydrate or solvate thereof.
